(19) United States
(12) Patent Application Publication
(10) Pub. No.: US 2007/0009886 A1
(43) Pub. Date: Jan. 11, 2007

(54) Carbohydrate-associated proteins

(75) Inventors: Narinder K. Chawla, Union City, CA (US); Y. Tom Tang, San Jose, CA (US); Jennifer A. Griffin, Fremont, CA (US); Yonghong G. Yang, San Jose, CA (US); Jayalaxmi Ramkumar, Fremont, CA (US); Reena Khare, Saratoga, CA (US); Thomas W. Richardson, Redwood City, CA (US); Shanya D. Becha, San Francisco, CA (US); Uyen K. Tran, San Jose, CA (US); Amy E. Kable, Silver Spring, MD (US); Anita Swarnakar, San Francisco, CA (US); Bridget A. Warren, San Marcos, CA (US); Vicki S. Elliott, San Jose, CA (US); Joseph P. Marquis, San Jose, CA (US); April A. Hafalia, Daly City, CA (US)

Correspondence Address: FOLEY AND LARDNER LLP;SUITE 500, 3000 K STREET NW, WASHINGTON, DC, 20007, US

(73) Assignee: Incyte Corporation

(21) Appl. No.: 10/534,578

(22) PCT Filed: Nov. 10, 2003
(86) PCT No.: PCT/US03/35947

§ 371 Date: Nov. 28, 2005

(30) Foreign Application Priority Data
Nov. 12, 2002 (US) 60425423
Jan. 21, 2003 (US) 60441847
Mar. 10, 2003 (US) 60453882
Mar. 20, 2003 (US) 60456645
Apr. 16, 2003 (US) 60463676

(51) Int. Cl. C12Q 1/68; G01N 33/53; C07H 21/04; C12P 21/06; C12N 9/24; C07K 16/40

(52) U.S. Cl. 435/6; 435/7.1; 435/69.1; 435/200; 435/320.1; 435/325; 530/388.26; 536/23.2

(57) ABSTRACT

Various embodiments of the invention provide human carbohydrate-associated proteins (CHOP) and polynucleotides which identify and encode CHOP. Embodiments of the invention also provide expression vectors, host cells, antibodies, agonists, and antagonists. Other embodiments provide methods for diagnosing, treating, or preventing disorders associated with aberrant expression of CHOP.

### TECHNICAL FIELD

The invention relates to novel nucleic acids, carbohydrate-associated proteins encoded by these nucleic acids, and to the use of these nucleic acids and proteins in the diagnosis, treatment, and prevention of carbohydrate metabolism, cell proliferative, autoimmune/inflammatory, reproductive, and neurological disorders. The invention also relates to the assessment of the effects of exogenous compounds on the expression of nucleic acids and carbohydrate-associated proteins.

### BACKGROUND OF THE INVENTION

Carbohydrates, including sugars or saccharides, starch, and cellulose, are aldehyde or ketone compounds with multiple hydroxyl groups. Carbohydrates have three important roles in mammalian cells. Carbohydrates function as energy-storage molecules, as fuels, and as metabolic intermediates. Carbohydrates are broken down to release energy in glycolysis or may be stored as glycogen for later use. The importance of carbohydrate metabolism is demonstrated by the sensitive regulatory system in place for maintenance of blood glucose levels in which two pancreatic hormones, insulin and glucagon, promote increased glucose uptake and storage by cells, and increased glucose release from cells, respectively. The sugars deoxyribose and ribose form part of the structural support of DNA and RNA, respectively, providing a second example of carbohydrate function. Third, carbohydrates provide a means for post-translational modification of secreted and membrane proteins and lipids. Indeed, 2-10% of the content of eukaryotic cell membranes is contributed by oligosaccharides on membrane glycoproteins and glycolipids. Carbohydrate modifications on glycoproteins and glycolipids create great structural diversity, and since they are mainly located on the extracellular side of the plasma membrane, they play an important role in intercellular recognition (Stryer, L. (1988) Biochemistry, W.H. Freeman and Company, New York N.Y., pp. 298-299, 331-347).

Proteins are associated with carbohydrates in several ways. Carbohydrate-containing macromolecules, which include glycoproteins, glycolipids, glycosaminoglycans, and proteoglycans, are found on the cell surface and in the extracellular matrix. The extracellular matrix is composed of diverse glycoproteins and carbohydrate-binding proteins which are secreted from the cell and assembled into an organized meshwork in close association with the cell surface. The interaction of the cell with the surrounding matrix profoundly influences cell shape, strength, flexibility, motility, and adhesion. These dynamic properties are intimately associated with signal transduction pathways controlling cell proliferation and differentiation, tissue construction, and embryonic development.

Glycoproteins have covalently attached carbohydrates which have been added to the proteins as they traverse the secretory pathway. Some proteins noncovalently associate with carbohydrate-containing macromolecules for purposes of binding, modifying, or degrading the carbohydrates. Glycoproteins include cell adhesion molecules, receptors, blood group antigens, growth factors, and antibodies. These proteins are involved in cellular processes such as cell-cell recognition and signaling, recognition and/or destruction of neurotransmitters, transmission of neural impulses, and immune function. Oligosaccharide modifications can provide great structural diversity. N— and O-linked oligosaccharides are transferred to proteins and modified in a series of enzymatic reactions that occur in the endoplasmic reticulum (ER) and Golgi. Oligosaccharides stabilize the protein during and after folding, orient the protein in the membrane, improve the protein's solubility, and act as a signal for lysosome targeting.

Heavily glycosylated glycoproteins are also referred to as proteoglycans. Proteoglycans in the extracellular matrix of connective tissues such as cartilage are essential for distributing the load in weight-bearing joints. Cell-surface-attached proteoglycans anchor cells to the extracellular matrix. Both extracellular and cell-surface proteoglycans bind growth factors, facilitating their binding to cell-surface receptors and subsequent triggering of signal transduction pathways (Lodish, H. et al. (1995) Molecular Cell Biology, Scientific American Books, New York N.Y., pp. 1139-1142).

Carbohydrates also form glycosaminoglycans (GAGs), which are linear unbranched polysaccharides composed of repetitive disaccharide units. GAGs exist free or as part of proteoglycans, large molecules composed of a core protein attached to one or more GAGs. GAGs are found on the cell surface, inside cells, and in the extracellular matrix. The GAG hyaluronan (HA) is found in the extracellular matrix of many cells, especially in soft connective tissues, and is abundant in synovial fluid (Pitsillides, A. A. et al. (1993) Int. J. Exp. Pathol. 74:27-34). HA, which functions in water and plasma protein homeostasis, seems to play important roles in cell regulation, development, and differentiation.

Glycolipids, along with phospholipids and cholesterol, form the membranes of cells. Examples of glycolipids include blood group antigens on erythrocytes and gangliosides in the myelin sheath of neurons. Modifications to glycoproteins and glycolipids on the extracellular side of the plasma membrane are important for intercellular recognition (Stryer, supra, pp. 298-299, 331-347; Lodish et al., supra, pp. 612-615).

Lectins are extracellular glycoproteins which bind cell surface carbohydrates specifically and reversibly, resulting in the agglutination of cells (Drickamer, K. and M. E. Taylor (1993) Annu. Rev. Cell Biol. 9:237-264). This function is particularly important for activation of the immune response. Lectins mediate the agglutination and mitogenic stimulation of lymphocytes at sites of inflammation (Lasky, L. A. (1991) J. Cell. Biochem. 45:139-146; Paietta, E. et al. (1989) J. Immunol. 143:2850-2857).

Lectins are classified into subfamilies based on carbohydrate-binding specificity. The galectin subfamily, in particular, includes lectins that bind β-galactoside carbohydrate moieties in a thiol-dependent manner (Hadari, Y. R. et al. (1995) J. Biol. Chem. 270:3447-3453). Galectins are widely expressed and developmentally regulated. Because all galectins lack an N-terminal signal peptide, it is suggested that galectins are externalized through an atypical secretory mechanism. Two classes of galectins have been defined based on molecular weight and oligomerization properties. Galectins contain a characteristic carbohydrate recognition domain (CRD), also known as a galaptin domain, which is about 140 amino acids long and contains several conserved residues (See Prosite PDOC00279 Vertebrate galactoside-binding lectin signature).

Another example is intelectin, a Ca²⁺ dependent lectin that binds to galactofuranosyl residues and bacterial arabinogalactan. Intelectin may play a role in the recognition of bacterial carbohydrate and induction of the immune response to microorganisms.

A comparison of recent structures of C-type lectin-like domains reveals diversity in the modular fold, particularly in the region associated with Ca2+ and sugar binding. Some of this diversity reflects the changes that occur during normal physiological functioning of the domains. C-type lectin-like domains associate with each other through several different surfaces to form dimers and trimers, from which ligand-binding sites project in a variety of different orientations (Drickamer, K.(1999) Curr. Opin. Struct. Biol. 9:585-590). The free CRD (group VII) lectins include HIP. HIP protein consists in a signal peptide linked to a carbohydrate-recognition domain (CRD), typical of C-type lectins without other binding domains. HIP, also known as the pancreatitis-associated protein (PAP). HIP/PAP is considered related to reg I alpha and reg I beta genes by descent from the same ancestral gene (Laserre, C. (1994) Eur. J. Biochem. 224:29-38). Reg I protein is a growth factor for pancreatic beta-cells of the islets of Langerhans, which produce insulin in humans. The Reg gene family also includes subclasses type II and III. Some of the type III Reg (Reg III) are implicated in the regeneration of cells other than pancreatic beta-cells, such as neuronal cells and epithelial cells in the alimentary tract (Okamoto H. (1999) J. Hepatobiliary Pancreat. Surg. 6:254-262).

Carbohydrate-Modifying Enzymes

The enzyme glutamine:fructose-6-phosphate amidotransferase (GFAT), also known as aminotransferase, catalyzes the reversible reaction of L-glutamine and D-fructose-6-phosphate to form L-glutamate and D-glucosamine-6-phosphate, which is the rate-limiting step in the hexosamine biosynthetic pathway (ExPASy ENZYME: EC 2.6.1.16). D-glucosamine-6-phosphate acts in the biosynthesis of UDP-N-acetyl-glucosamine (UDP-GlcNAc) and other hexosamines incorporated into glycoproteins and proteoglycans. GFAT regulates the availability of precursors for N— and O-linked glycosylation. Glucosamine enhances the production of transforming growth factor (TGF)-β1 (Kolm-Litty, V. et al. (1998) J. Clin. Invest. 101:160-169). GFAT activity plays a role in insulin resistance in Type II diabetes, and GFAT overexpression leads to insulin resistance. Hexosaamine metabolism appears to regulate glycogen synthase, the rate-limiting enzyme in glycogen synthesis, as well as PP1G, a glycogen-bound protein phosphatase, pyruvate kinase, and the glucose transporter GLUT1 (McClain, D. A. and E. D. Crook (1996) Diabetes 45:1003-1009).

The enzyme glucosamine-6-phosphate deaminase (GNPDA), also known as isomerase, catalyzes the reversible reaction of D-glucosamine-6-phosphate with water to form D-fructose-6-phosphate and ammonia (ExPASy ENZYME EC 5.3.1.10). This reaction links hexosamine systems with glycolytic pathways and may provide an energy source from the catabolism of hexosamines in glycoproteins, glycolipids, and sialic-acid-containing macromolecules. GNPDA is expressed in tissues with high energy requirements (Wolosker, H. et al. (1998) FASEB J. 12:91-99).

The enzyme UDP-glucose dehydrogenase (UDPGD) catalyzes the reversible reaction of UDP-glucose, 2 NAD⁺, and water to form UDP-glucuronate and 2 NADH (ExPASy ENZYME EC 1.1.1.22). UDP-glucuronate is needed for the biosynthesis of GAGs, which appear to play a role in signal transduction pathways (Binari, R. C. et al. (1997) Development 124:2623-2632).

Man₉-mannosidase is an α1,2-mannosidase (glycosyl hydrolase) involved in the early processing of N-linked oligosaccharides. This enzyme catalyzes the specific cleavage of α1,2-mannosidic linkages in Man₉-(GlcNAc)₂ and Man₅-(GlcNAc)₂. Multiple α1,2-mannosidases have been identified in mammalian cells and may be needed for the processing of distinct classes of N-glycoproteins. Man₉-mannosidase is a Type II membrane protein with a short cytoplasmic tail, a single transmembrane domain, and a large luminal catalytic domain. The human kidney enzyme is localized to the Golgi (Bause, E. et al. (1993) Eur. J. Biochem. 217:535-540; Bieberich, E. and Bause, E. (1995) Eur. J. Biochem. 233:644-649).

DPM1 is an enzyme in the endoplasmic reticulum that catalyzes the production of dolichol phosphate-mannose (DPM) from GDP-mannose and dolichol phosphate. The activity of DPM1 is regulated by DPM2, which targets DPM1 to the endoplasmic reticulum (ER) and increases its affinity for dolichol phosphate. DMP2 resides in the (ER) membrane and contains two putative transmembrane domains and a putative ER-localization signal near its C-terminus.

Glycosylation

Glycosylation refers to the covalent attachment of any number of carbohydrate chains (oligosaccharides) to specific sites (glycosylation sites) on proteins. Glycosylation is a post-translational protein modification essential to the conformation, stability, transport, secretion, antigenicity, clearance and activity of glycosylated proteins (glycoproteins). The composition of the attached oligosaccharides is specific to a protein and may be simple (consisting primarily of mannose residues) or complex (with additional N-acetyl-glucosamine (GlcNAc), sialic acid, and galactose residues). Glycoproteins may have relatively few carbohydrate groups or may contain a larger percentage of carbohydrate than protein (based on molecular weight). These latter, heavily glycosylated glycoproteins are also referred to as proteoglycans to emphasize the predominant carbohydrate composition of the molecules. The type of saccharide bond (e.g., α, β, 1,2-, 1,4-) formed between any two constituent carbohydrate residues is also a critical molecular determinant for the structure and function of the glycoprotein.

Glycosylation confers increased hydrophilicity to proteins. Many glycoproteins, such as carrier proteins, antibodies, and lysosomal proteins, are found free in solution (e.g., plasma). Other glycoproteins are membrane-bound. In the case of membrane-associated glycoproteins, the carbohydrate side chain serves to orient the glycoproteins in the membrane lipid bilayer. The glycosylated regions of the molecule interact with the aqueous environment on the inside or outside of the membrane while the more hydrophobic domains of the glycoprotein (typically consisting of non-polar amino acid residues that are not glycosylated) interact with the phospholipids in the membrane.

Addition of oligosaccharide side chains occurs at the —NH₂ group of asparagine (Asn) residues (N-linked glycosylation) or at the —OH group of serine (Ser) residues (O-linked glycosylation). The process of N-linked glycosylation begins in the endoplasmic reticulum (ER) and is completed in the Golgi apparatus of eukaryotic cells. O-linked glycosylation occurs exclusively in the Golgi. Of all characterized glycoproteins, 90% are N-glycosylated, with or without additional O-glycosylation. Only 10% are exclusively O-glycosylated (Apweiler, R. et al. (1999) Biochim. Biophys. Acta 1473:4-8). Almost two-thirds of the approximately 75,000 SWISS-PROT protein sequences include putative N-glycosylation sites, underscoring the importance of this protein modification in nature (Apweiler et al., supra). Biochemical steps involved in N-linked glycosylation have been well characterized, and are reviewed below.

N-Linked Glycosylation

N-linkage of carbohydrates to proteins occurs via a nitrogen atom of asparagine (Asn) residue side chains in the context of the primary amino acid sequence Asn-X-Ser or Asn-X-Thr (Ser=serine, Thr=threonine, and X=any amino acid residue except proline). While the composition of N-linked oligosaccharides is highly diverse, the pathways responsible for glycosylation have common first steps. A 14-residue core oligosaccharide, containing two N-acetylglucosamine (GlcNAc), nine mannose, and three glucose residues, is transferred as a unit from a dolichol phosphate donor molecule to the —NH₂ group of an acceptor Asn residue on the target protein. Typically, the three glucose residues of the core oligosaccharide are removed by glucosidases I and II resulting in “high mannose oligosaccharides” side chains. These partially processed N-linked glycoproteins are then sequentially transported from the ER through the cis-, medial-, and trans-cisternae of the Golgi (Bonay, P. et al. (1996) J. Biol. Chem. 271:3719-3726). Further modification to the oligosaccharide chains may occur to remove additional core mannose residues using the enzymes Golgi mannosidase I (cis-cisterna), N-acetyl-glucosaminyltransferase (GluNAcT; medial-cisterna), and Golgi mannosidase II (trans-cisternae). Following the removal of some of the mannose residues by Golgi mannosidase I, the addition of a single GlcNAc by GluNAcT is essential for the removal of the remaining mannose residues of the core oligosaccharide by Golgi mannosidase II.

Mannose-1-phosphate guanyltransferases are involved in early steps of protein glycosylation. They participate in sugar metabolism and their enzymatic products are channeled into glycoprotein synthesis. Mannose-1-phosphate guanyltransferase 1 (MPG1), also referred to as NDP-hexose pyrophosphorylase, catalyzes the conversion of GTP and αD-mannose 1-phosphate into diphosphate and CDP-ethanolamine in mannose metabolism. This enzyme is very similar to CDP-glucose pyrophosphorylase and may also be involved in the regulation of cell cycle progression. A cDNA coding for GTP:αD-mannose-1-phosphate guanyltransferase 1 (MPG1) was recently isolated from a cDNA library of a Trichoderma reesei strain (Kruszewska, J. S. et al. (1998) Curr. Genet. 33:445-450). The nucleotide sequence of the 1.6 kb cDNA revealed an ORF which encodes a protein of 364 amino acids. Sequence comparisons demonstrate 70% identity with the yeast Saccharomyces cerevisiae guanyltransferase gene 1 (MPG1) and 75% identity with the Schizosaccharomyces pombe homolog.

Complex oligosaccharide side chains result from the addition of N-acetyl-glucosamine, N-acetylneuraminic acid (sialic acid), and galactose, as well as other sugar moieties, to the remaining core sugar moieties on the partially-processed glycoprotein. These modifications occur in the trans-cisterna and trans-Golgi network (TGN), and involve a number of enzymes including N-acetyl-glucosaminyltransferase I (GlcNAcTsI), sialyltransferases (STs), and galactosyltransferases (GalTs). Multiple isoforms of many of these enzymes produce specific α or β, 1,2-, 1,3-, 1,4-, or 1,6-disaccharide bonds between constituent sugar residues of the oligosaccharide side chain. The stereochemistry and type of bonds in a carbohydrate side chain contribute to the overall structure and function of the resulting glycoprotein (Lehninger, A. L. et al. (1993) Principles of Biochemistry, Worth Publishers, New York N.Y., pp. 931; Lewin, B. (1997) Genes VI, Oxford University Press, New York N.Y., pp. 1030-1033).

Galactosyltransferases are a subset of glycosyltransferases that transfer galactose (Gal) to the terminal N-acetylglucosamine (GlcNAc) oligosaccharide chains that are part of glycoproteins or glycolipids that are free in solution (Kolbinger, F. et al. (1998) J. Biol. Chem. 273:433-440; Amado, M. et al. (1999) Biochim. Biophys. Acta 1473:35-53). Galactosyltransferases are found in the Golgi, on the cell surface, and as soluble extracellular proteins, in addition to being present in the Golgi. β1,3-galactosyltransferases form Type I carbohydrate chains with Gal (β1-3)GlcNAc linkages. β1,3-galactosyltransferases appear to have a short cytosolic domain, a single transmembrane domain, and a catalytic domain with eight conserved regions (Kolbinger et al., supra; Hennet, T. et al. (1998) J. Biol. Chem. 273:58-65). In mouse, UDP-galactose:β-N-acetylglucosamine β1,3-galactosyltransferase-I region 1 is located at amino acid residues 78-83, region 2 is located at amino acid residues 93-102, region 3 is located at amino acid residues 116-119, region 4 is located at amino acid residues 147-158, region 5 is located at amino acid residues 172-183, region 6 is located at amino acid residues 203-206, region 7 is located at amino acid residues 236-246, and region 8 is located at amino acid residues 264-275. A variant of a sequence found within mouse UDP-galactose:β-N-acetylglucosamine β1,3-galactosyltransferase-I region 8 is also found in bacterial galactosyltransferases, suggesting that this sequence defines a galactosyltransferase sequence motif (Hennet et al., supra). Recent work suggests that brainiac protein is a β1,3-galactosyltransferase (Yuan, Y. et al. (1997) Cell 88:9-11; Hennet et al., supra).

UDP-Gal:GlcNAc-1,4-galactosyltransferase (-1,4-GalT) catalyzes the formation of Type II carbohydrate chains with Gal (β1-4)GlcNAc linkages (Sato, T. et al. (1997) EMBO J. 16:1850-1857). A soluble form of the enzyme is formed by cleavage of the membrane-bound form. Amino acids conserved among β1,4-galactosyltransferases include two cysteines linked through a disulfide-bond and a putative UDP-galactose binding site in the catalytic domain (Yadav, S. P. and K. Brew (1990) J. Biol. Chem. 265:14163-14169; Yadav, S. P. and K. Brew (1991) J. Biol. Chem. 266:698-703; Shaper, N. L. et al. (1997) J. Biol. Chem. 272:31389-31399). β1,4-galactosyltransferases have several specialized roles in addition to synthesizing carbohydrate chains on glycoproteins or glycolipids. A β1,4-galactosyltransferase functions as part of a heterodimer with α-lactalbumin in mammary lactose production. A β1,4-galactosyltransferase on the surface of sperm functions as a receptor that specifically recognizes the egg. Cell surface β1,4-galactosyltransferases also function in cell adhesion, cell recognition, cell/basal lamina interaction, and normal and metastatic cell migration (Shur, B. (1993) Curr. Opin. Cell Biol. 5:854-863; Shaper, J. (1995) Adv. Exp. Med. Biol. 376:95-104; Masri, K. A et al. (1988) Biochem. Biophys. Res. Commun. 157:657-663).

Synthetases are another class of carbohydrate-modifying enzymes that have critical roles in proper cell funtioning. For example, production of sialylated glycoconjugates requires the synthesis of cytidine 5′-monophosphate N-acetylneuraminic acid (CMP-Neu5Ac or CMP-sialic acid), a reaction catalyzed by CMP-Neu5Ac synthetase (Munster, A. K. et al. (1998) Proc. Natl. Acad. Sci. USA 95:9140-9145). Sialic acids of cell surface glycoproteins and glycolipids contribute to proper structure and function in a variety of tissues. Sialyltransferases (STs) are a subset of glycosyltransferases that catalyze the transfer of sialic acid (from CMP-sialic acids) to the carbohydrate groups of glycoproteins and glycolipids. A variety of these Type II membrane proteins are present in the Golgi. Cloned members of this gene family share an N-terminal cytoplasmic tail region, a transmembrane region, and a large luminal region containing three sialyl motifs designated large (L), small (S), and very small (VS). The L-sialyl motif contributes to donor substrate binding and consists of eight invariant residues within a highly conserved stretch of 48-49 amino acids. The 23-amino acid S-sialyl motif contributes to the binding of both donor and acceptor substrates (Datta, A. et al. (1997) Indian J. Biochem. Biophys. 34:157-165). In the case of a representative sialytransferase ST3GalI (˜350 amino acids in length), the L, S, and VS regions correspond to amino acids 138-182, 264-286, and 309-321, respectively. Other cloned members of the family include ST6GalNAcI and ST8SiaI. ST6GalNAcI is larger than the other known sialyltransferases, and is composed of more than 500 amino acid residues (Tsuji, S. et al. (1996) Glycobiology (letter) 6:v-vii; Geremia, R. et al. (1997) Glycobiology (letter) 7:v-vii; Datta, A. et al. (1995) J. Biol. Chem. 270:1497-1500; Datta, A. et al. (1998) J. Biol. Chem. 273:9608-9618; Tsuji, S. et al. (1998) J. Biochem. 120:1-13). Sialyltransferases are not abundant in cellular extracts, but several have been cloned and expressed. At least one inhibitor has been synthesized (Horenstein, B. et al. (1996) J. Am. Chem. Soc. 118:10371-10379).

Lysosomal apyrase-like protein of 70 kDa (LALP70) belongs to the apyrase or GDA1/CD39 family and is almost identical to a human uridine diphosphatase, with the exception of nine extra amino acids in LALP70. The apyrase protein family comprises enzymes capable of cleaving nucleotide tri- and diphosphates (NTP-diphosphohydrolase activity) in a calcium or magnesium-independent manner, thus modulating the ratio between the cellular levels of nucleoside diphosphates and nucleoside triphosphates. Members of this family were originally described as ectoenzymes, with some intracellular exceptions. Evidence from hydrophobicity analysis of the encoded protein and other experiments revealed a transmembrane region at the N— and C-termini, and suggest that LALP70 is a type III lysosomal/autophagic vacuole membrane protein with the apyrase conserved regions facing the luminal space of the vacuoles (Biederbick, A. et al. (1999) J. Cell Sci. 112:2473-2484). The first splice variant described in the apyrase protein family was discovered in LALP70. Comparison of the enzymatic properties of the splice variants revealed a broader substrate specificity for hLALP70v with CTP, UDP, CDP, GTP, and GDP as preferred substrates, while hLALP70 utilized UTP and TTP preferentially (Biederbick A. et al. (2000) J. Biol. Chem. 275:19018-19024).

A variety of other enzymes that are involved in sugar metabolism participate directly or indirectly in glycosylation, upstream of events that occur in the ER and Golgi. Many of these enzymes were originally identified in bacteria and plants and are less well characterized in humans; however, human homologs may exist that perform similar functions. For example, ADP-glucose pyrophosphorylases catalyze a very important step in the biosynthesis of α1,4-glucans (glycogen or starch) in bacteria and plants, namely the synthesis of the activated glucosyl donor, ADP-glucose, from glucose-1-phosphate and ATP. ADP-glucose pyrophosphorylases are tetrameric, allosterically-regulated enzymes. There are a number of conserved regions in the sequence of bacterial and plant ADP-glucose pyrophosphorylase subunits. Additionally, there are three regions which are considered signature patterns. The first two regions are N-terminal and have been proposed to be part of the allosteric and substrate-binding sites in the Escherichia coli enzyme. The third pattern corresponds to a conserved region in the central part of the enzymes.

Carbohydrate Metabolism Disorders

Carbohydrate metabolism is altered in several disorders. Diabetes mellitus is characterized by abnormally high blood glucose (hyperglycemia). Type I diabetes results from an autoimmune-related loss of pancreatic insulin-secreting cells. Type II diabetes results from insulin resistance and impaired insulin secretory response to glucose, and is associated with obesity. Hypoglycemia, or abnormally low blood glucose levels, has several causes including drug use, genetic deficiencies in carbohydrate metabolism enzymes, cancer, liver disease, and renal disease (Berkow, R. et al. (1992) The Merck Manual of Diagnosis and Therapy, Internet Edition, Section 8, Chapter 91, Diabetes Mellitus, Hypoglycemia).

Mutations in enzymes involved in protein glycosylation cause severe diseases. For example, alpha mannosidase mutations cause congenital dyserythropoietic anemia Type I and alpha B lysosomal mannosidosis (Isselbacher, K. J. et al. (1994) Harrison's Principles of Internal Medicine, McGraw-Hill, Inc. New York N.Y., pp. 2092-2093; and Online Mendelian Inheritance In Man, 224100).

Glucosidases represent another class of carbohydrate-modifying enzymes that catalyze the release of glucose from carbohydrates through hydrolysis of the glycosidic link in various glucosides. The inherited disorder type I Gaucher disease, which is characterized by hematologic abnormalities, can be detected in a heterozygous or homozygous individual through an assay of leukocyte beta-glucosidase levels (Raghavan, S. S. et al. (1980) Am. J. Hum. Genet. 32:158-173). Patients with all three types of Gaucher disease exhibit a deficiency of an enzyme called glucocerebrosidase that catalyzes the first step in the biodegradation of glucocerebroside. In the brain, glucocerebroside arises from the turnover of complex lipids during brain development and the formation of the myelin sheath of nerves. In other tissues, glucocerebroside arises mainly from the biodegradation of old red and white blood cells.

Galectins play a number of roles in diseases and conditions associated with cell-cell and cell-matrix interactions. For example, certain galectins associate with sites of inflammation and bind to cell surface immunoglobulin E molecules. In addition, galectins may play an important role in cancer metastasis. Galectin overexpression is correlated with the metastatic potential of cancers in humans and mice. Moreover, anti-galectin antibodies inhibit processes associated with cell transformation, such as cell aggregation and anchorage-independent growth.

Galectin-8, also known as prostate carcinoma tumor antigen 1 (PCTA-1), is a novel galectin implicated in cancer progression (Su, Z.-Z. et al. (1996) Proc. Natl. Acad. Sci. USA 93:7252-7257). Galectin-8 is expressed in invasive prostate carcinomas and early-stage prostate cancers, but not in normal prostate or benign prostatic hypertrophic tissue.

Defects in carbohydrate metabolism are also associated with cancer. Reduced GAG and proteoglycan expression is associated with human lung carcinomas (Nackaerts, K. et al. (1997) Int. J. Cancer 74:335-345). The carbohydrate determinants sialyl-LewisA and sialyl-LewisX are frequently expressed on human cancer cells. These determinants, ligands for the cell adhesion molecule E-selectin, are involved in the adhesion of cancer cells to vascular endothelium and contribute to hematogenous metastasis of cancer (Kannagi, R. (1997) Glycoconj. J. 14:577-584). Alterations of the N-linked carbohydrate core structure of cell surface glycoproteins are linked to colon and pancreatic cancers (Schwarz, R. E. et al. (1996) Cancer Lett. 107:285-291). Reduced expression of the Sda blood group carbohydrate structure in cell surface glycolipids and glycoproteins is observed in gastrointestinal cancer (Dohi, T. et al. (1996) Int. J. Cancer 67:626-631).

Changes in glycosaminoglycan levels are associated with several autoimmune diseases. Both increases and decreases in various GAGs occur in patients with autoimmune thyroid disease and autoimmune diabetes mellitus. Antibodies to GAGs were found in patients with systemic lupus erythematosus and autoimmune thyroid disease (Hansen, C. et al. (1996) Clin. Exp. Rheum. 14:S59-S67). The glycosaminoglycan hyaluronan (HA) induces tumor cell adhesion and migration, and its small fragments are angiogenic. Serum HA is diagnostic of liver disease and various inflammatory conditions, such as rheumatoid arthritis. Interstitial edema caused by accumulation of HA may cause dysfunction in various organs (Laurent, T. C. and J. R. Fraser (1992) FASEB J. 6:2397-2404). Hyaluronidase is an enzyme that degrades HA to oligosaccharides by catalyzing the random hydrolysis of 1,4-linkages between N-acetyl-β-D-glucosamine and D-glucuronate residues. Hyaluronidases may function in cell adhesion, infection, angiogenesis, and signal transduction. Hyaluronidases are associated with reproduction, cancer, and inflammation. Hyaluronidase activity is significantly elevated in prostate tumor tissue compared to that in both normal prostate and benign prostate hyperplasia (Lokeshwar, V. B. et al. (1996) Cancer Res. 56:651-657).

PH-20, a protein expressed in the mammalian testis and present on the plasma membrane of mouse and human sperm, has hyaluronidase activity (Lin, Y. et al. (1994) J. Cell Biol. 125:1157-63). PH-20 enables sperm to penetrate the mammalian egg's outer layer, which consists of about 3,000 cumulus cells embedded in an extracellular matrix rich in HA. Penetration of this layer is an essential step in the fertilization process. PH-20 is also expressed in some tumor cells. Non-testicular mammalian hyaluronidases include the HYAL1 hyaluronidase, expressed in human serum, and lysosomal hyaluronidase HYAL2, expressed in many cells (Lepperdinger, G. et al. (1998) J. Biol. Chem. 273:22466-22470). HYAL2 may have a role in producing distinct HA fragments that can induce angiogenesis and the expression of enzymes involved in signal transduction pathways, such as nitric oxide synthase. A lysosomal-type hyaluronidase may degrade HA in lung fibroblasts in a cytokine-regulated process (Sampson, P. M. et al. (1992) J. Clin. Invest. 90:1492-1503). The venom of numerous animals including various snakes, bees, hornets, stone fish, platypus, scorpions, and lizards contain hyaluronidase. Venom hyaluronidase is thought to act as an aid in the diffusion of toxins.

A number of human diseases are linked to genetic or acquired deficiencies in protein glycosylation. Carbohydrate-deficient glycoprotein syndromes (CDGSs) include a host of alterations in glycosylation in a number of disorders and diseases. CDGSs are a group of hereditary multisystem disorders (Matthijs, G. et al. (1997) Nat. Genet. 16:88-92) causing severe psychomotor and mental retardation, as well as blood coagulation abnormalities seen in thrombosis, bleeding, or stroke-like episodes. The characteristic biochemical abnormality of CDGSs is the hypoglycosylation (N-linked) of glycoproteins (Freeze, H. and Aebi, M. (1999) Biochim. Biophys. Acta 1455:167-78). Depending on the type of CDGS, the carbohydrate side chains of glycoproteins are either truncated or completely missing from the protein core. Several different types of CDGS have been classified. The most common form, CDGS type 1A, is caused by phosphomannomutase (PMM1) deficiency (Matthijs, G. (1998) Am. J. Hum. Genet. 62:542-50). PMM1 functions upstream of MPG1 (see above) and catalyzes the conversion of D-mannose-6-phosphate to D-mannose-1-phosphate, which is required for the initial steps of protein glycosylation.

A second form of CDGS, designated CDGS type 1B, has also been described (Niehues, R. et al. (1998) J. Clin. Invest. 101:1414-1420). Psychomotor dysfunction and mental retardation are not present in this disease; instead, CDGS type 1B is a gastrointestinal disorder characterized by protein-losing enteropathy, severe hypoglycemia, vomiting, diarrhea, and congenital hepatic fibrosis. Nonetheless, some patients who are affected with CDGS type 1B suffer from thrombosis and life-threatening bleeding. A deficiency of phosphomannose isomerase (PMI) was identified as the most likely cause of this syndrome. Most symptoms can be controlled with dietary mannose supplements (Niehues et al., supra; Freeze and Aebi, supra). This form of CDGS is the first inherited disorder in human metabolism that shows a decrease in available mannose.

Defects in glucosyltransferase function also play an important role in some human diseases. Galactosyltransferases may be involved in autoimmune/inflammatory disorders as many humans with autoimmune thyroid disorders have high levels of circulating antibodies directed against the enzymatic product of α1,3-galactosyltransferase (Etienne-Decerf, J. et al. (1987) Acta Endocrinol. 115:67-74). An aberrantly-cleaved, soluble β1,4-galactosyltransferase is secreted by a human ovarian cancer cell line (Uejima, T. et al. (1992) Cancer Res. 52:6158-6163). β1,4-GalT-deficient transgenic mice exhibited growth retardation in one experiment (Asano, M. et al. (1997) EMBO J. 16:1850-1857), while targeted inactivation of the mouse α1,4-GalT in another study was usually lethal (Furukawa, K. et al. (1999) Biochim. Biophys. Acta. 1473:54-66). In a third study, the constitutive overexpression of an α1,3-galactosyltransferase in transgenic mice led to the increased secretion of proteins in the urine, low body weight, partial damage to hair growth, and early death (Ikematsu, S. et al. (1999) Glycoconj. J. 1999 16:73-76). Galactosyltransferases have also been implicated in the regulation of cellular growth, development, and differentiation and may play an important role in embryogenesis as well as tumor development. Secreted galactosyltransferases, derived in some cases from proteolytic cleavage of membrane-bound forms, may trigger cell surface receptors by binding their bound carbohydrates or may modify carbohydrates on cell surface molecules in a regulated fashion. Extracellular carbohydrate moieties are developmentally regulated and are likely involved in the regulation of cell migration (Shur, B. et al. (1984) Mol. Cell. Biochem 61:143-158; Paulson, J. and K. Colley (1989) J. Biol. Chem. 264:17615-17618). The expression of β1,6-GlcNAc-bearing N-linked glycoproteins has been used as a marker of tumor progression in human breast and colon cancer, and astrocytes from human glioma specimens were found to contain increased levels of these types of glycoproteins compared to astrocytes from normal individuals (Yamamoto, H. et al. (2000) Cancer Res. 2000 60:134-142). These observations suggest that the dysfunction of another isoform of a glucosyltransferase, a β1,6-GlcNAcT, may also play a role in tumor formation or invasivity.

Sialyltransferases have also been implicated in human disease. Elevated levels of 2,6-sialyltransferase (but not 2,3-sialyltransferase) are detected in human choriocarcinoma tissues, apparently the result of upregulation at the transcriptional level (Fukushima, K. (1998) Cancer Res. 58:4301-4306). Transient transfection of 2,6-sialyltransferase into human, tumorigenic, glioma cell line reduces the invasivity of the cells (Yamamoto, H. (1997) J. Neurochem. 68:2566-2576). Chronic alcohol (ethanol) consumption causes a decrease in Gal-β1,4-GlcNAc-α2,6-sialyltransferase (α2,6-ST) activity in the livers of rats who obtained at least one-third of their calories from alcohol for a period of one month or longer. Liver α2,6-ST activity returned to normal after a week of abstinence from alcohol consumption. Based on the results of nuclear run-on assays and mRNA stability assays, the reduction in α2,6-ST activity was the result of a 50% decrease in the half-life of α2,6-ST mRNA (Rao, M. (1999) Metabolism 48:797-803). A significant decrease in the plasma α2,6-sialyltransferase activity was also observed in a group of individuals suffering from clinical depression. This particular form of depression was attributed to a change in glucocorticoid receptor (GR) functionality. These findings suggested that α2,6-ST enzyme or activity levels may be a contributing factor in clinical depression or at least a useful biochemical marker of cortisol receptor dysfunction (Maguire, T. et al. (1997) Biological Psychiatry 41:1131-1136).

Additional human diseases that involve defects in glycosylation, and the enzyme deficiencies that cause them, include (i) aspartylglycosaminuria, an aspartylglycosaminidase deficiency that causes mental retardation, (ii) GM₁ and GM₂ gangliosidosis, β-galactosidase and β-N-acetylhexosaminidase deficiencies, respectively, that cause glycolipid storage diseases, (iii) α-mannosidosis and β-mannosidosis, caused by a deficiency of α-mannosidase or β-mannosidase, respectively, that cause neurological dysfunction, and (iv) sialidosis, caused by a neuraminidase deficiency, characterized by hepatosplenomegaly as well as impaired neural development.

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Breast Cancer

More than 180,000 new cases of breast cancer are diagnosed each year, and the mortality rate for breast cancer approaches 10% of all deaths in females between the ages of 45-54 (Gish, K. (1999) AWIS Magazine 28:7-10). However the survival rate based on early diagnosis of localized breast cancer is extremely high (97%), compared with the advanced stage of the disease in which the tumor has spread beyond the breast (22%). Current procedures for clinical breast examination are lacking in sensitivity and specificity, and efforts are underway to develop comprehensive gene expression profiles for breast cancer that may be used in conjunction with conventional screening methods to improve diagnosis and prognosis of this disease (Perou, C. M. et al. (2000) Nature 406:747-752).

Mutations in two genes, BRCA1 and BRCA2, are known to greatly predispose a woman to breast cancer and may be passed on from parents to children (Gish, supra). However, this type of hereditary breast cancer accounts for only about 5% to 9% of breast cancers, while the vast majority of breast cancer is due to non-inherited mutations that occur in breast epithelial cells.

The relationship between expression of epidermal growth factor (EGF) and its receptor, EGFR, to human mammary carcinoma has been particularly well studied. (See Khazaie, K. et al. (1993) Cancer and Metastasis Rev. 12:255-274, and references cited therein for a review of this area.) Overexpression of EGFR, particularly coupled with down-regulation of the estrogen receptor, is a marker of poor prognosis in breast cancer patients. In addition, EGFR expression in breast tumor metastases is frequently elevated relative to the primary tumor, suggesting that EGFR is involved in tumor progression and metastasis. This is supported by accumulating evidence that EGF has effects on cell functions related to metastatic potential, such as cell motility, chemotaxis, secretion and differentiation. Changes in expression of other members of the erbB receptor family, of which EGFR is one, have also been implicated in breast cancer. The abundance of erbB receptors, such as HER-2/neu, HER-3, and HER-4, and their ligands in breast cancer points to their functional importance in the pathogenesis of the disease, and may therefore provide targets for therapy of the disease (Bacus, S. S. et al. (1994) Am. J. Clin. Pathol. 102:S13-S24). Other known markers of breast cancer include a human secreted frizzled protein mRNA that is downregulated in breast tumors; the matrix G1a protein which is overexpressed in human breast carcinoma cells; Drg1 or RTP, a gene whose expression is diminished in colon, breast, and prostate tumors; maspin, a tumor suppressor gene downregulated in invasive breast carcinomas; and CaN19, a member of the S100 protein family, all of which are down-regulated in mammary carcinoma cells relative to normal mammary epithelial cells (Zhou, Z. et al. (1998) Int. J. Cancer 78:95-99; Chen, L. et al. (1990) Oncogene 5:1391-1395; Ulrix, W. et al (1999) FEBS Lett 455:23-26; Sager, R. et al. (1996) Curr. Top. Microbiol. Immunol. 213:51-64; and Lee, S. W. et al. (1992) Proc. Natl. Acad. Sci. USA 89:2504-2508).

Cell lines derived from human mammary epithelial cells at various stages of breast cancer provide a useful model to study the process of malignant transformation and tumor progression as it has been shown that these cell lines retain many of the properties of their parental tumors for lengthy culture periods (Wistuba, I. I. et al. (1998) Clin. Cancer Res. 4:2931-2938). Such a model is particularly useful for comparing phenotypic and molecular characteristics of human mammary epithelial cells at various stages of malignant transformation.

Lung Cancer

Lung cancer is the leading cause of cancer death in the United States, affecting more than 100,000 men and 50,000 women each year. Nearly 90% of the patients diagnosed with lung cancer are cigarette smokers. Tobacco smoke contains thousands of noxious substances that induce carcinogen metabolizing enzymes and covalent DNA adduct formation in the exposed bronchial epithelium. In nearly 80% of patients diagnosed with lung cancer, metastasis has already occurred. Most commonly lung cancers metastasize to pleura, brain, bone, pericardium, and liver. The decision to treat with surgery, radiation therapy, or chemotherapy is made on the basis of tumor histology, response to growth factors or hormones, and sensitivity to inhibitors or drugs. With current treatments, most patients die within one year of diagnosis. Earlier diagnosis and a systematic approach to identification, staging, and treatment of lung cancer could positively affect patient outcome.

Lung cancers progress through a series of morphologically distinct stages from hyperplasia to invasive carcinoma. Malignant lung cancers are divided into two groups comprising four histopathological classes. The Non Small Cell Lung Carcinoma (NSCLC) group includes squamous cell carcinomas, adenocarcinomas, and large cell carcinomas and accounts for about 70% of all lung cancer cases. Adenocarcinomas typically arise in the peripheral airways and often form mucin secreting glands. Squamous cell carcinomas typically arise in proximal airways. The histogenesis of squamous cell carcinomas may be related to chronic inflammation and injury to the bronchial epithelium, leading to squamous metaplasia. The Small Cell Lung Carcinoma (SCLC) group accounts for about 20% of lung cancer cases. SCLCs typically arise in proximal airways and exhibit a number of paraneoplastic syndromes including inappropriate production of adrenocorticotropin and anti-diuretic hormone.

Lung cancer cells accumulate numerous genetic lesions, many of which are associated with cytologically visible chromosomal aberrations. The high frequency of chromosomal deletions associated with lung cancer may reflect the role of multiple tumor suppressor loci in the etiology of this disease. Deletion of the short arm of chromosome 3 is found in over 90% of cases and represents one of the earliest genetic lesions leading to lung cancer. Deletions at chromosome arms 9p and 17p are also common. Other frequently observed genetic lesions include overexpression of telomerase, activation of oncogenes such as K-ras and c-myc, and inactivation of tumor suppressor genes such as RB, p53 and CDKN2.

Genes differentially regulated in lung cancer have been identified by a variety of methods. Using mRNA differential display technology, Manda et al. (1999; Genomics 51:5-14) identified five genes differentially expressed in lung cancer cell lines compared to normal bronchial epithelial cells. Among the known genes, pulmonary surfactant apoprotein A and alpha 2 macroglobulin were down regulated whereas nm23H1 was upregulated. Petersen et al. (2000; Int. J. Cancer, 86:512-517) used suppression subtractive hybridization to identify 552 clones differentially expressed in lung tumor derived cell lines, 205 of which represented known genes. Among the known genes, thrombospondin-1, fibronectin, intercellular adhesion molecule 1, and cytokeratins 6 and 18 were previously observed to be differentially expressed in lung cancers. Wang et al. (2000; Oncogene 19:1519-1528) used a combination of microarray analysis and subtractive hybridization to identify 17 genes differentially overexpresssed in squamous cell carcinoma compared with normal lung epithelium Among the known genes they identified were keratin isoform 6, KOC, SPRC, IGFb2, connexin 26, plakofillin 1 and cytokeratin 13.

Inflammation and Immune Responses

Human peripheral blood mononuclear cells (PBMCs) represent the major cellular components of the immune system. PBMCs contain about 12% B lymphocytes, 25% CD4+ and 15% CD8+ lymphocytes, 20% NK cells, 25% monocytes, and 3% various cells that include dendritic cells and progenitor cells. The proportions, as well as the biology of these cellular components tend to vary slightly between healthy individuals, depending on factors such as age, gender, past medical history, and genetic background. PBMCs are useful for studying the effects of various inflammatory mediators and immune response proteins on gene expression. Some examples of these molecules are described below.

Interleukin 1 beta (IL-1β) is a cytokine associated with acute inflammatory responses and is generally considered the prototypical pro-inflammatory cytokine. However, IL-1β functions are not limited to the inflammatory response since this molecule is involved in processes such as fever induction, metabolic regulation, and bone remodeling. Both cells of the immune system (monocytes, dendritic cells, NK cells, platelets, and neutrophils) and somatic cells (osteoblasts, neurons, Schwann's cells, oligodendrocytes, and adrenal cortical cells) can produce IL-1β. IL-1β has been shown to induce its own production in monocytes; induce the production of adhesion molecules and chemokines in endothelial cells; and in conjunction with IL-12, induce interferon-γ production by NK Cells. IL-1β is produced as a single chain pro-molecule that needs to be cleaved by a specialized protease, IL-1β Converting Enzyme (ICE), to acquire its function.

Interleukin 2 (IL-2) is a protein with a variety of immunologic functions, most notably the ability to promote the proliferation and maturation of activated T cells. Some of the biological activities attributed to IL-2 include: induction of secretion of IFN-γ and TNF-α and -β from PBMCs; stimulation of the rate of synthesis of c-myc RNA and transferrin receptor; activation of neutrophils; stimulation of proliferation and maturation of activated helper T cells; stimulation of proliferation of activated and natural killer cells and tumor-infiltrating lymphocytes, as well as enhancement of the ability to kill target cells; induction of IL-2 receptor expression on T cells; and stimulation of antibody-producing B cell proliferation.

Interleukin 3 (IL-3) is a pleiotropic factor produced primarily by activated T cells that can stimulate the proliferation and differentiation of pluripotent hematopoietic stem cells and various lineage committed progenitors. IL-3 also affects the functional activity of mature mast cells, basophils, eosinophils, and macrophages. Because of its multiple functions and targets, IL-3 was originally studied under different names, including mast cell growth factor, P-cell stimulating factor, burst promoting activity, multi-colony stimulating factor, thy-1 inducing factor, and WEHI-3 growth factor. In addition to activated T cells, other cell types such as human thymic epithelial cells, activated murine mast cells, murine keratinocytes, and neurons/astrocytes can also produce IL-3. IL-3 exerts its biological activities by binding to specific cell surface receptors. The high affinity receptor responsible for IL-3 signaling is composed of at least two subunits, an IL-3 specific α-chain that binds IL-3 with low affinity and a common β-chain that is shared by the IL-5 and GM-CSF highaffinity receptors. Although the β-chain itself does not bind IL-3, it confers high-affinity IL-3 binding in the presence of the α-chain. Receptors for IL-3 are present on bone marrow progenitors, macrophages, mast cells, eosinophils, megakaryocytes, basophils, and various myeloid leukemic cells.

Interleukin 4 (IL-4) is a pleiotropic cytokine produced by activated T cells, mast cells, and basophils. It was initially identified as a B cell differentiation factor (BCDF) and a B cell stimulatory factor (BSF1). Subsequent to the molecular cloning and expression of both human and mouse IL-4, numerous other functions have been ascribed to B cells and other hematopoietic and non-hematopoietic cells including T lymphocytes, monocytes, macrophages, mast cells, myeloid and erythroid progenitors, fibroblasts, endothelial cells, etc. IL-4 exhibits anti-tumor effects both in vivo and in vitro. Recently, IL-4 was identified as an important regulator for the CD4+ subset (Th1-like vs. Th2-like) development. The biological effects of IL-4 are mediated by the binding of IL-4 to specific cell surface receptors. The functional high-affinity receptor for IL-4 consists of a ligand-binding subunit (IL-4R) and a second subunit (β chain) that can modulate the ligand binding affinity of the receptor complex. In certain cell types, the gamma chain of the IL-2 receptor complex is a functional β chain of the IL-4 receptor complex. Signaling of IL-4 through its receptor leads to the activation of Signal Transducer and Activator of Transcription 6 (STAT6).

Interleukin 5 (IL-5) is a T cell-derived factor that promotes the proliferation, differentiation, and activation of eosinophils. IL-5 has also been known as T cell replacing factor (TRF), B cell growth factor II (BCGFII), B cell differentiation factor m (BCDF m), eosinophil differentiation factor (EDF), and eosinophil colony-stimulating factor (Eo-CSF). IL-5 exerts its activity on target cells by binding to specific cell surface receptors. The functional high-affinity receptor for human IL-5 is composed of a low-affinity IL-5 binding α-subunit and a non-binding common β-subunit that is shared with the high-affinity receptors for GM-CSF and IL-3.

Interleukin 6 (IL-6) is a multifunctional protein that plays important roles in host defense, acute phase reactions, immune responses, and hematopoiesis. According to the type of biological responses being studied, IL-6 was previously named interferon-b2, 26-kDa protein, B cell stimulatory factor-2 (BSF-2), hybridoma/plasmacytoma growth factor, hepatocyte stimulating factor, cytotoxic T cell differentiation factor, and macrophage-granulocyte inducing factor 2A (MGI-2A). The IL-6 designation was adopted after these variously named proteins were found to be identical on the basis of their amino acid and/or nucleotide sequences. IL-6 is expressed by a variety of normal and transformed cells including T cells, B cells, monocytes/macrophages, fibroblasts, hepatocytes, keratinocytes, astrocytes, vascular endothelial cells, and various tumor cells. The production of IL-6 is upregulated by numerous signals including mitogenic or antigenic stimulation, LPS, calcium ionophore, IL-1, IL-2, IFN, TNF, PDGF, and viruses. IL-4 and IL-13 inhibit IL-6 expression in monocytes.

Interleukin 7 (IL-7), previously known as pre-B-cell growth factor and lymphopoietin-1, was originally purified on the basis of its ability to promote the proliferation of precursor B-cells. It has been shown that IL-7 can also stimulate the proliferation of thymocytes, T cell progenitors, and mature CD4+ and CD8+ T cells. IL-7 can induce the formation of lymphokine-activated killer (LAK) cells as well as the development of cytotoxic T lymphocytes (CTL). Among myeloid lineage cells, IL-7 can upregulate the production of pro-inflammatory cytokines and stimulate the tumoricidal activity of monocytes/macrophages. IL-7 is expressed by adherent stromal cells from various tissues. IL-7 bioactivities are mediated by the binding of IL-7 to functional high-affinity receptor complexes. The ligand binding subunit (IL-7R) of the IL-7 receptor complex has been cloned from human and mouse sources. Recently, the γ chain of the IL-2 receptor complex has been shown to be an essential component for IL-7 signal transduction. Both IL-7R and IL-2Rγ are members of the hematopoietin receptor superfamily. Cells known to express IL-7 receptors include pre-B cells, T cells, and bone marrow cells.

Interleukin 8 (IL-8) was originally discovered and purified independently by a number of laboratories as a neutrophil chemotactic and activating factor. It was also referred to as neutrophil chemotactic factor (NCF), neutrophil activating protein (NAP), monocyte-derived neutrophil chemotactic factor (MDNCF), T-lymphocyte chemotactic factor (TCF), granulocyte chemotactic protein (GCP), and leukocyte adhesion inhibitor (LAI). Many cell types, including monocyte/macrophages, T cells, neutrophils, fibroblasts, endothelial cells, keratinocytes, hepatocytes, chondrocytes, and various tumor cell lines can produce IL-8 in response to a wide variety of pro-inflammatory stimuli such as exposure to IL-1, TNF, LPS, and viruses. IL-8 is a member of the alpha (C—X—C) subfamily of chemokines, which also includes platelet factor 4, GRO, IP-10, etc. IL-8 is a potent chemoattractant for neutrophils and has a wide range of other pro-inflammatory effects. IL-8 causes degranulation of neutrophil-specific granules and azurophilic granules. IL-8 induces expression of the cell adhesion molecules CD11/CD18 and enhances the adherence of neutrophils to endothelial cells and sub-endothelial matrix proteins. Besides neutrophils, IL-8 is also chemotactic for basophils, T cells, and eosinophils. IL-8 has been reported to be a co-mitogen for keratinocytes and was also shown to be an autocrine growth factor for melanoma cells. Recently, IL-8 was reported to be angiogenic both in vivo and in vitro.

Interleukin 10 (IL-10), initially designated cytokine synthesis inhibitory factor (CSIF), was originally identified as a product of murine T helper 2 (Th2) clones that inhibited the cytokine production by Th1 clones, which are dependent upon stimulation with antigen in the presence of antigen presenting cells (APC). The human homolog of murine IL-10 was subsequently cloned by cross-hybridization. Human IL-10 is produced by CD4+ T cell clones as well as by some CD8+ T cell clones. In addition, human B cells, EBV-transformed lymphoblastoid cell lines, and monocytes can also produce IL-10 upon activation. IL-10 is a pleiotrophic cytokine that can exert either immunostimulatory or immunosupressive effects on a variety of cell types. It is a potent immunosuppressant of macrophage functions. In vitro, IL-10 can inhibit the accessory function and antigen-presenting capacity of monocytes by, among other effects, downregulating class II MHC expression. Thus, IL-10 can inhibit monocyte/macrophage-dependent, antigen-specific proliferation of mouse Th1 clones as well as human Th0-, Th1-, and Th2-like T cells. IL-10 can also inhibit the monocyte/macrophage-dependent, antigen stimulated cytokine synthesis (especially IFN-γ) by human PBMC and NK cells. Additionally, IL-10 is a potent inhibitor of monocyte/macrophage activation and its resultant cytotoxic effects. It can suppress the production of numerous cytokines including TNF-α, IL-1, IL-6, and IL-10, as well as the synthesis of superoxide anion, reactive oxygen intermediates, and reactive nitrogen intermediates by activated monocytes/macrophages. As an immunostimulatory cytokine, IL-10 can act on B cells to enhance their viability, cell proliferation, Ig secretion, and class II MHC expression. Aside from B lymphocytes, IL-10 is also a growth co-stimulator for thymocytes and mast cells, as well as an enhancer of cytotoxic T cell development.

Interleukin 12 (IL-12), also known as natural killer cell stimulatory factor (NKSF) or cytotoxic lymphocyte maturation factor (CLMF), is a pleiotropic cytokine originally identified in the medium of activated human B lymphoblastoid cell lines. IL-12 is produced by macrophages and B lymphocytes and has been shown to have multiple effects on T cells and natural killer (NK) cells. These include inducing production of IFN-γ and TNF by resting and activated T and NK cells, enhancing the cytotoxic activity of resting NK and T cells, inducing and synergizing with IL-2 in the generation of lyrnphokine-activated killer (LAK) cells, acting as a comitogen to stimulate proliferation of resting T cells, and inducing proliferation of activated T and NK cells. Current evidence indicates that IL-12, produced by macrophages in response to infectious agents, is a central mediator of the cell-mediated immune response by its actions on the development, proliferation, and activities of Th1 cells. In its role as the initiator of cell-mediated immunity, it has been suggested that IL-12 has therapeutic potential as a stimulator of cell-mediated immune responses to microbial pathogens, metastatic cancers, and viral infections such as AIDS.

Interleukin 18 (IL-18), also known as interferon-gamma-inducing factor (IGIF) and IL-1γ, is a recently described cytokine that shares some biologic activities with IL-12 and structural similarities with the IL-1 family of proteins. IL-18 was originally cloned from liver cells and has since been shown to be expressed by monocyte/macrophages, osteoblasts, and keratinocytes. Human IL-18 cDNA encodes a 193 amino acid residue biologically inactive precursor molecule (pro-IL-18) that requires cleavage by a specific protease—ICE—to acquire its function. Like IL-12, human IL-18 has been shown to enhance NK cell activity in PBMC cultures. Human IL-18 has also been found to induce the production of IFN-γ and GM-CSF while inhibiting the production of IL-10 by PBMCs. On enriched human T cells, human IL-18 can enhance Th1 cytokine production and stimulate cell proliferation via an IL-2-dependent pathway.

Granulocyte Colony Stimulating Factor (G-CSF) is a pleiotropic cytokine best known for its specific effects on the proliferation, differentiation, and activation of hematopoietic cells of the neutrophilic granulocyte lineage. Activated monocytes and macrophages are the primary sources of G-CSF in the body. Fibroblasts, endothelial cells, astrocytes, and bone marrow stromal cells can also produce this cytokine upon activation. In vitro, G-CSF stimulates growth, differentiation, and functions of cells from the neutrophil lineage. Consistent with its in vitro functions, G-CSF plays important roles in defending against infection, in inflammation and repair, and in maintaining steady state hematopoiesis.

Granulocyte-Monocyte Colony Stimulating Factor (GM-CSF) Granulocyte-monocyte colony stimulating factor (GM-CSF) was first described as a factor that can support the in vitro colony formation of granulocyte-macrophage progenitors. In addition, GM-CSF is a growth factor for erythroid, megakaryocyte, and eosinophil progenitors. Lymphocytes (T and B), monocytes, macrophages, mast cells, endothelial cells, and fibroblasts can produce GM-CSF upon activation. GM-CSF exerts its biological effects by binding to specific cell surface receptors. The high affinity receptors required for human GM-CSF signal transduction are heterodimers consisting of a GM-CSF-specific α chain and a common β chain that is shared by the high-affinity receptors for IL-3 and IL-5.

Interferon gamma (IFN-γ), also known as Type II interferon or immune interferon, is a cytokine produced primarily by T-lymphocytes and natural killer cells. IFN-γ was originally characterized based on its antiviral activities. The protein also exerts antiproliferative, immunoregulatory, and proinflammatory activities and is thus important in host defense mechanisms. IFN-γ induces the production of cytokines and upregulates the expression of class I and II MHC antigens, Fc receptor, and leukocyte adhesion molecules. It modulates macrophage effector functions, influences isotype switching, and potentiates the secretion of immunoglobulins by B cells. IFN-γ also augments Th1 cell expansion and may be required for Th1 cell differentiation. The IFN-γ receptor is present on almost all cell types except mature erythrocytes and has been cloned and characterized. The IFN-γ receptor is structurally related to the recently cloned IL-10 receptor.

Leptin is a protein product of the mouse obesity gene. Mice with mutations in the obesity gene that block the synthesis of leptin tend to be obese and diabetic and exhibit reduced activity, metabolism, and body temperature. Human leptin shares approximately 84% sequence identity with the mouse protein. Human leptin cDNA encodes a 167-amino acid residue protein with a 21-amino acid residue signal sequence that is cleaved to yield the 146-amino acid residue mature protein. The expression of leptin mRNA is restricted to adipose tissue. A high-affinity receptor for leptin (OB—R) with homology to gp130 and the G-CSF receptor has recently been cloned. OB—R mRNA is expressed in the choroid plexus and in the hypothalamus. OB—R is also an isoform of B219, a sequence that is expressed in at least four isoforms in very primitive hematopoietic cell populations and in a variety of lymphohematopoietic cell lines. The possible roles of leptin in body weight regulation, hematopoiesis, and reproduction are being investigated.

Leukemia inhibitory factor (LIF) was initially identified as a factor that inhibits the proliferation and induces the differentiation to macrophages of the murine myeloid leukemic cell line M1. Subsequent to its purification and molecular cloning, LIF was recognized to be a pleiotropic factor with multiple effects on both hematopoietic and non-hematopoietic cells. LIF has overlapping biological functions with OSM, IL-6, IL-11, and CNTF. All these cytokines use gp130 as a component in their signal transducing receptor complexes. Human LIF cDNA encodes a 202 amino acid residue polypeptide with a 22-amino acid residue signal peptide that is cleaved to yield a 180-amino acid residue mature human LIF.

Tumor Growth Factor beta (TGF-β) is a stable, nultifunctional polypeptide growth factor. While specific receptors for this protein have been found on almost all mammalian cell types thus far examined, the effect of the molecule varies depending on the cell type and growth conditions. Generally, TGF-β is stimulatory for cells of mesenchymal origin and inhibitory for cells of epithelial or neuroectodermal origin. TGF-β has been found in the highest concentration in human platelets and mammalian bone, but is produced by many cell types in smaller amounts.

Tumor necrosis factor alpha (TGF-α), also called cachectin, is produced by neutrophils, activated lymphocytes, macrophages, NK cells, LAK cells, astrocytes, endothelial cells, smooth muscle cells, and some transformed cells. TNF-α occurs as a secreted, soluble form and as a membrane-anchored form, both of which are biologically active. Two types of receptors for TNF-α have been described and virtually all cell types studied show the presence of one or both of these receptor types. TNF-α and TNF-β are extremely pleiotropic factors due to the ubiquity of their receptors, to their ability to activate multiple signal transduction pathways, and to their ability to induce or suppress the expression of a wide number of genes. TNF-α and TGF-β play a critical role in mediation of the inflammatory response and in mediation of resistance to infections and tumor growth.

Steroid Hormones

The potential application of gene expression profiling is particularly relevant to measuring the toxic response to potential therapeutic compounds and of the metabolic response to therapeutic agents. Diseases treated with steroids and disorders caused by the metabolic response to treatment with steroids include adenomatosis, cholestasis, cirrhosis, hemangioma, Henoch-Schonlein purpura, hepatitis, hepatocellular and metastatic carcinomas, idiopathic thrombocytopenic purpura, porphyria, sarcoidosis, and Wilson disease. Response may be measured by comparing both the levels and sequences expressed in tissues from subjects exposed to or treated with steroid compounds such as mifepristone, progesterone, beclomethasone, medroxyprogesterone, budesonide, prednisone, dexamethasone, betamethasone, or danazol with the levels and sequences expressed in normal untreated tissue.

Steroids are a class of lipid-soluble molecules, including cholesterol, bile acids, vitamin D, and hormones, that share a common four-ring structure based on cyclopentanoperhydrophenanthrene and that carrry out a wide variety of functions. Cholesterol, for example, is a component of cell membranes that controls membrane fluidity. It is also a precursor for bile acids which solubilize lipids and facilitate absorption in the small intestine during digestion. Vitamin D regulates the absorption of calcium in the small intestine and controls the concentration of calcium in plasma. Steroid hormones, produced by the adrenal cortex, ovaries, and testes, include glucocorticoids, mineralocorticoids, androgens, and estrogens. They control various biological processes by binding to intracellular receptors that regulate transcription of specific genes in the nucleus. Glucocorticoids, for example, increase blood glucose concentrations by regulation of gluconeogenesis in the liver, increase blood concentrations of fatty acids by promoting lipolysis in adipose tissues, modulate sensitivity to catcholamines in the central nervous system, and reduce inflammation. The principal mineralocorticoid, aldosterone, is produced by the adrenal cortex and acts on cells of the distal tubules of the kidney to enhance sodium ion reabsorption. Androgens, produced by the interstitial cells of Leydig in the testis, include the male sex hormone testosterone, which triggers changes at puberty, the production of sperm and maintenance of secondary sexual characteristics. Female sex hormones, estrogen and progesterone, are produced by the ovaries and also by the placenta and adrenal cortex of the fetus during pregnancy. Estrogen regulates female reproductive processes and secondary sexual characteristics. Progesterone regulates changes in the endometrium during the menstrual cycle and pregnancy.

Steroid hormones are widely used for fertility control and in anti-inflammatory treatments for physical injuries and diseases such as arthritis, asthma, and auto-immune disorders. Progesterone, a naturally occurring progestin, is primarily used to treat amenorrhea, abnormal uterine bleeding, or as a contraceptive. Endogenous progesterone is responsible for inducing secretory activity in the endometrium of the estrogen-primed uterus in preparation for the implantation of a fertilized egg and for the maintenance of pregnancy. It is secreted from the corpus luteum in response to luteinizing hormone (LH). The primary contraceptive effect of exogenous progestins involves the suppression of the midcycle surge of LH. At the cellular level, progestins diffuse freely into target cells and bind to the progesterone receptor. Target cells include the female reproductive tract, the mammary gland, the hypothalamus, and the pituitary. Once bound to the receptor, progestins slow the frequency of release of gonadotropin releasing hormone from the hypothalamus and blunt the pre-ovulatory LH surge, thereby preventing follicular maturation and ovulation. Progesterone has minimal estrogenic and androgenic activity. Progesterone is metabolized hepatically to pregnanediol and conjugated with glucuronic acid.

Medroxyprogesterone (MAH), also known as 6α-methyl-17-hydroxyprogesterone, is a synthetic progestin with a pharmacological activity about 15 times greater than progesterone. MAH is used for the treatment of renal and endometrial carcinomas, amenorrhea, abnormal uterine bleeding, and endometriosis associated with hormonal imbalance. MAH has a stimulatory effect on respiratory centers and has been used in cases of low blood oxygenation caused by sleep apnea, chronic obstructive pulmonary disease, or hypercapnia.

Mifepristone, also known as RU-486, is an antiprogesterone drug that blocks receptors of progesterone. It counteracts the effects of progesterone, which is needed to sustain pregnancy. Mifepristone induces spontaneous abortion when administered in early pregnancy followed by treatment with the prostaglandin, misoprostol. Further, studies show that mifepristone at a substantially lower dose can be highly effective as a postcoital contraceptive when administered within five days after unprotected intercourse, thus providing women with a “morning-after pill” in case of contraceptive failure or sexual assault. Mifepristone also has potential uses in the treatment of breast and ovarian cancers in cases in which tumors are progesterone-dependent. It interferes with steroid-dependent growth of brain meningiomas, and may be useful in treatment of endometriosis where it blocks the estrogen-dependent growth of endometrial tissues. It may also be useful in treatment of uterine fibroid tumors and Cushing's Syndrome. Mifepristone binds to glucocorticoid receptors and interferes with cortisol binding. Mifepristone also may act as an anti-glucocorticoid and be effective for treating conditions where cortisol levels are elevated such as AIDS, anorexia nervosa, ulcers, diabetes, Parkinson's disease, multiple sclerosis, and Alzheimer's disease.

Danazol is a synthetic steroid derived from ethinyl testosterone. Danazol indirectly reduces estrogen production by lowering pituitary synthesis of follicle-stimulating hormone and LH. Danazol also binds to sex hormone receptors in target tissues, thereby exhibiting anabolic, antiestrognic, and weakly androgenic activity. Danazol does not possess any progestogenic activity, and does not suppress normal pituitary release of corticotropin or release of cortisol by the adrenal glands. Danazol is used in the treatment of endometriosis to relieve pain and inhibit endometrial cell growth. It is also used to treat fibrocystic breast disease and hereditary angioedema.

Corticosteroids are used to relieve inflammation and to suppress the immune response. They inhibit eosinophil, basophil, and airway epithelial cell function by regulation of cytokines that mediate the inflammatory response. They inhibit leukocyte infiltration at the site of inflammation, interfere in the function of mediators of the inflammatory response, and suppress the humoral immune response. Corticosteroids are used to treat allergies, asthma, arthritis, and skin conditions. Beclomethasone is a synthetic glucocorticoid that is used to treat steroid-dependent asthma, to relieve symptoms associated with allergic or nonallergic (vasomotor) rhinitis, or to prevent recurrent nasal polyps following surgical removal. The anti-inflammatory and vasoconstrictive effects of intranasal beclomethasone are 5000 times greater than those produced by hydrocortisone. Budesonide is a corticosteroid used to control symptoms associated with allergic rhinitis or asthma. Budesonide has high topical anti-inflammatory activity but low systemic activity. Dexamethasone is a synthetic glucocorticoid used in anti-inflammatory or immunosuppressive compositions. It is also used in inhalants to prevent symptoms of asthma. Due to its greater ability to reach the central nervous system; dexamethasone is usually the treatment of choice to control cerebral edema. Dexamethasone is approximately 20-30 times more potent than hydrocortisone and 5-7 times more potent than prednisone. Prednisone is metabolized in the liver to its active form, prednisolone, a glucocorticoid with anti-inflammatory properties. Prednisone is approximately 4 times more potent than hydrocortisone and the duration of action of prednisone is intermediate between hydrocortisone and dexamethasone. Prednisone is used to treat allograft rejection, asthma, systemic lupus erythematosus, arthritis, ulcerative colitis, and other inflammatory conditions. Betamethasone is a synthetic glucocorticoid with antiinflammatory and immunosuppressive activity and is used to treat psoriasis and fungal infections, such as athlete's foot and ringworm

The anti-inflammatory actions of corticosteroids are thought to involve phospholipase A₂ inhibitory proteins, collectively called lipocortins. Lipocortins, in turn, control the biosynthesis of potent mediators of inflammation such as prostaglandins and leukotrienes by inhibiting the release of the precursor molecule arachidonic acid. Proposed mechanisms of action include decreased IgE synthesis, increased number of β-adrenergic receptors on leukocytes, and decreased arachidonic acid metabolism. During an immediate allergic reaction, such as in chronic bronchial asthma, allergens bridge the IgE antibodies on the surface of mast cells, which triggers these cells to release chemotactic substances. Mast cell influx and activation, therefore, is partially responsible for the inflammation and hyperirritability of the oral mucosa in asthmatic patients. This inflammation can be retarded by administration of corticosteroids.

The effects upon liver metabolism and hormone clearance mechanisms are important to understand the pharmacodynamics of a drug. The human C3A cell line is a clonal derivative of HepG2/C3 (hepatoma cell line, isolated from a 15-year-old male with liver tumor), which was selected for strong contact inhibition of growth. The use of a clonal population enhances the reproducibility of the cells. C3A cells have many characteristics of primary human hepatocytes in culture: i) expression of insulin receptor and insulin-like growth factor II receptor; ii) secretion of a high ratio of serum albumin compared with α-fetoprotein; iii) conversion of ammonia to urea and glutamine; iv) metabolize aromatic amino acids; and v) proliferate in glucose-free and insulin-free medium. The C3A cell line is now well established as an in vitro model of the mature human liver (Mickelson, J. K. et al. (1995) Hepatology 22:866-875; Nagendra, A. R. et al. (1997) Am. J. Physiol. 272:G408-G416).

There is a need in the art for new compositions, including nucleic acids and proteins, for the diagnosis, prevention, and treatment of carbohydrate metabolism, cell proliferative, autoimmune/inflammatory, reproductive, and neurological disorders.

### SUMMARY OF THE INVENTION

Various embodiments of the invention provide purified polypeptides, carbohydrate-associated proteins, referred to collectively as ‘CHOP’ and individually as ‘CHOP-1,’ ‘CHOP-2,’ ‘CHOP-3,’ ‘CHOP-4,’ ‘CHOP-5,’ ‘CHOP-6,’ ‘CHOP-7,’ ‘CHOP-8,’ ‘CHOP-9,’ ‘CHOP-10,’ ‘CHOP-11,’ ‘CHOP-12,’ ‘CHOP-13,’ ‘CHOP-14,’ ‘CHOP-15,’ ‘CHOP-16,’ ‘CHOP-17,’ ‘CHOP-18,’ ‘CHOP-19,’ and ‘CHOP-20’ and methods for using these proteins and their encoding polynucleotides for the detection, diagnosis, and treatment of diseases and medical conditions. Embodiments also provide methods for utilizing the purified carbohydrate-associated proteins and/or their encoding polynucleotides for facilitating the drug discovery process, including determination of efficacy, dosage, toxicity, and pharmacology. Related embodiments provide methods for utilizing the purified carbohydrate-associated proteins and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. Another embodiment provides an isolated polypeptide comprising an amino acid sequence of SEQ ID NO:1-20.

Still another embodiment provides an isolated polynucleotide encoding a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. In another embodiment, the polynucleotide encodes a polypeptide selected from the group consisting of SEQ ID NO:1-20. In an alternative embodiment, the polynucleotide is selected from the group consisting of SEQ ID NO:21-40.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20.

Still yet another embodiment provides an isolated polynucleotide selected from the group consisting of a) a polynucleotide comprising a polynucleotide sequence selected from the group consisting of SEQ ID NO:21-40, b) a polynucleotide comprising a naturally occurring polynucleotide sequence at least 90% identical or at least about 90% identical to a polynucleotide sequence selected from the group consisting of SEQ ID NO:21-40, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:21-40, b) a polynucleotide comprising a naturally occurring polynucleotide sequence at least 90% identical or at least about 90% identical to a polynucleotide sequence selected from the group consisting of SEQ ID NO:21-40, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:21-40, b) a polynucleotide comprising a naturally occurring polynucleotide sequence at least 90% identical or at least about 90% identical to a polynucleotide sequence selected from the group consisting of SEQ ID NO:21-40, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amnino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20, and a pharmaceutically acceptable excipient In one embodiment, the composition can comprise an amino acid sequence selected from the group consisting of SEQ ID NO:1-20. Other embodiments provide a method of treating a disease or condition associated with decreased or abnormal expression of functional CHOP, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) contacting a sample comprising the polypeptide with a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional CHOP, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) contacting a sample comprising the polypeptide with a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional CHOP, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:21-40, the method comprising a) contacting a sample comprising the target polynucleotide with a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:21-40, ii) a polynucleotide comprising a naturally occurring polynucleotide sequence at least 90% identical or at least about 90% identical to a polynucleotide sequence selected from the group consisting of SEQ ID NO:21-40, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:21-40, ii) a polynucleotide comprising a naturally occurring polynucleotide sequence at least 90% identical or at least about 90% identical to a polynucleotide sequence selected from the group consisting of SEQ ID NO:21-40, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

Table 8 shows single nucleotide polymorphisms found in polynucleotide sequences of the invention, along with allele frequencies in different human populations.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

Definitions

“CHOP” refers to the amino acid sequences of substantially purified CHOP obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of CHOP. Agonists may include proteins, nucleic acids, carbohydrates, small molecules, or any other compound or composition which modulates the activity of CHOP either by directly interacting with CHOP or by acting on components of the biological pathway in which CHOP participates.

An “allelic variant” is an alternative form of the gene encoding CHOP. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding CHOP include those sequences with deletions, insertions, or substitutions of different nucleotides, resulting in a polypeptide the same as CHOP or a polypeptide with at least one functional characteristic of CHOP. Included within this definition are polymorphisms which may or may not be readily detectable using a particular oligonucleotide probe of the polynucleotide encoding CHOP, and improper or unexpected hybridization to allelic variants, with a locus other than the normal chromosomal locus for the polynucleotide encoding CHOP. The encoded protein may also be “altered,” and may contain deletions, insertions, or substitutions of amino acid residues which produce a silent change and result in a functionally equivalent CHOP. Deliberate amino acid substitutions may be made on the basis of one or more similarities in polarity, charge, solubility, hydrophobicity, hydrophilicity, and/or the amphipathic nature of the residues, as long as the biological or immunological activity of CHOP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of CHOP. Antagonists may include proteins such as antibodies, anticalins, nucleic acids, carbohydrates, small molecules, or any other compound or composition which modulates the activity of CHOP either by directly interacting with CHOP or by acting on components of the biological pathway in which CHOP participates.

The term “antibody” refers to intact immunoglobulin molecules as well as to fragments thereof, such as Fab, F(ab′)₂, and Fv fragments, which are capable of binding an epitopic determinant. Antibodies that bind CHOP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic CHOP, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding CHOP or fragments of CHOP may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (Accelrys, Burlington Mass.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of CHOP or a polynucleotide encoding CHOP which can be, identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:21-40 can comprise a region of unique polynucleotide sequence that specifically identifies SEQ ID NO:21-40, for example, as distinct from any other sequence in the genome from which the fragment was obtained. A fragment of SEQ ID NO:21-40 can be employed in one or more embodiments of methods of the invention, for example, in hybridization and amplification technologies and in analogous methods that distinguish SEQ ID NO:21-40 from related polynucleotides. The precise length of a fragment of SEQ ID NO:21-40 and the region of SEQ ID NO:21-40 to which the fragment corresponds are routinely determinable by one of ordinary skill in the art based on the intended purpose for the fragment.

A fragment of SEQ ID NO:1-20 is encoded by a fragment of SEQ ID NO:21-40. A fragment of SEQ ID NO:1-20 can comprise a region of unique amino acid sequence that specifically identifies SEQ ID NO:1-20. For example, a fragment of SEQ ID NO:1-20 can be used as an immunogenic peptide for the development of antibodies that specifically recognize SEQ ID NO:1-20. The precise length of a fragment of SEQ ID NO:1-20 and the region of SEQ ID NO:1-20 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, alternatively, sequence identity, between two or more polynucleotide sequences or two or more polypeptide sequences.

The terms “percent identity” and “% identity,” as applied to polynucleotide sequences, refer to the percentage of identical nucleotide matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:

Matrix: BLOSUM62

Reward for match: 1

Penalty for mismatch: −2

Open Gap: 5 and Extension Gap: 2 penalties

Gap x drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of identical residue matches between at least two polypeptide sequences aligned using a standardized algorithm Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. The phrases “percent similarity” and “% similarity,” as applied to polypeptide sequences, refer to the percentage of residue matches, including identical residue matches and conservative substitutions, between at least two polypeptide sequences aligned using a standardized algorithm. In contrast, conservative substitutions are not included in the calculation of percent identity between polypeptide sequences.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:

Matrix: BLOSUM62

Open Gap: 11 and Extension Gap: 1 penalties

Gap x drop-off: 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. and D. W. Russell (2001; Molecular Cloning: A Laboratory Manual, 3rd ed., vol. 1-3, Cold Spring Harbor Press, Cold Spring Harbor N.Y., ch. 9).

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of CHOP which is capable of eliciting an immune response when introduced into a living organism, for example, a mammal. The term “immunogenic fragment” also includes any polypeptide or oligopeptide fragment of CHOP which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of CHOP. For example, modulation may cause an increase or a decrease in protein activity, binding characteristics, or any other biological, functional, or immunological properties of CHOP.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an CHOP may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of CHOP.

“Probe” refers to nucleic acids encoding CHOP, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in, for example, Sambrook, J. and D. W. Russell (2001; Molecular Cloning: A Laboratory Manual, 3rd ed., vol. 1-3, Cold Spring Harbor Press, Cold Spring Harbor N.Y.), Ausubel, F. M. et al. (1999; Short Protocols in Molecular Biology, 4^{th} ed., John Wiley & Sons, New York N.Y.), and Innis, M. et al. (1990; PCR Protocols. A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook and Russell (supra). The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing CHOP, nucleic acids encoding CHOP, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook and Russell (supra).

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity or sequence similarity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity or sequence similarity over a certain defined length of one of the polypeptides.

The Invention

Various embodiments of the invention include new human carbohydrate-associated proteins (CHOP), the polynucleotides encoding CHOP, and the use of these compositions for the diagnosis, treatment, or prevention of carbohydrate netabolism, cell proliferative, autoimmune/inflammatory, reproductive, and neurological disorders.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to the polypeptide and polynucleotide sequences of the invention. The full length clones encode polypeptides which have at least 95% sequence identity to the polypeptide sequences shown in column 3.

Table 2 shows sequences with homology to polypeptide embodiments of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows amino acid residues comprising signature sequences, domains, motifs, potential phosphorylation sites, and potential glycosylation sites. Column 5 shows analytical methods for protein structure/function analysis and in some cases, searchable databases to which the analytical methods were applied.

Together, Tables 2 and 3 summarize the properties of polypeptides of the invention, and these properties establish that the claimed polypeptides are carbohydrate-associated proteins. For example, SEQ ID NO:2 is 50% identical, from residue T20 to residue L617, to Drosophila melanogaster phosphomannomutase 45A (GenBank ID g16797814) as determined by the Basic Local Alignment Search Tool (BLAST). (See Table 2.) The BLAST probability score is 1.9e-155, which indicates the probability of obtaining the observed polypeptide sequence alignment by chance. SEQ ID NO:2 also has homology to proteins that are isomerases, and proteins that are members of the phosphoglucomutase and phsophomannomutase family, as determined by BLAST analysis using the PROTEOME database. SEQ ID NO:2 also contains a phosphoglucomutase/phosphomannomutase, alpha/beta/alpha domain I domain as determined by searching for statistically significant matches in the hidden Markov model (HMM)-based PFAM database of conserved protein families/domains. (See Table 3.) Data from BLIMPS and MOTIFS analyses, and BLAST analyses against the PRODOM and DOMO databases, provide further corroborative evidence that SEQ ID NO:2 is a phosphomannomutase.

As another example, SEQ ID NO:8 is 99% identical, from residue V43 to residue E370, to human mDC-SIGN2 type I isoform (GenBank ID g15383606) as determined by the Basic Local Alignment Search Tool (BLAST). (See Table 2.) The BLAST probability score is 7.4e-174, which indicates the probability of obtaining the observed polypeptide sequence alignment by chance. SEQ ID NO:8 also has homology to proteins that are localized to the plasma membrane, have acts as a receptor for ICAM3, binds strains of HIV-1 and HIV-2, and are members of the C-type lectin family, as determined by BLAST analysis using the PROTEOME database. SEQ ID NO:8 also contains a C-type lectin domain as determined by searching for statistically significant matches in the hidden Markov model (HMM)-based PFAM and SMART databases of conserved protein families/domains. (See Table 3.) Data from BLIMPS and MOTIFS analyses, and BLAST analyses against the DOMO database, provide further corroborative evidence that SEQ ID NO:8 is a member of the C-type lectin family.

As another example, SEQ ID NO:10 is 98% identical, from residue M1 to residue D213, 100% identical, from residue D212 to residue M371, and 97% identical, from residue R373 to residue A415, to human ERGL (GenBank ID g11120502) as determined by the Basic Local Alignment Search Tool (BLAST). (See Table 2.) The BLAST probability score is 3.2e-203, which indicates the probability of obtaining the observed polypeptide sequence alignment by chance. SEQ ID NO:10 also has homology to proteins that are localized to the Golgi and endoplasmic reticulum, termed mannose-binding lectin 1, which are involved in the traffic of glycoproteins between endoplasmic reticulum and the Golgi apparatus; mutations of the corresponding gene is associated with combined factor V and VIII coagulation deficiency, as determined by BLAST analysis using the PROTEOME database. SEQ ID NO:10 also contains a Legume-like lectin family domain as determined by searching for statistically significant matches in the hidden Markov model (HMM)-based PFAM database of conserved protein families/domains. (See Table 3.) Data from BLAST analyses against the PRODOM and DOMO databases, provide further corroborative evidence that SEQ ID NO:10 is a carbohydrate-associated protein.

As another example, SEQ ID NO:18 is a splice variant of human lung surfactant protein D (GenBank ID g34767) as determined by the Basic Local Alignment Search Tool (BLAST). (See Table 2.) The BLAST probability score is 2.1e-138, which indicates the probability of obtaining the observed polypeptide sequence alignment by chance. SEQ ID NO:18 also has homology to small molecule-binding glycoproteins, proteins that are C-type lectins, and proteins that play a role in neutralizing infections due to inhaled microorganisms, as determined by BLAST analysis using the PROTEOME database. SEQ ID NO:18 also contains a C-type lectin (CTL) or carbohydrate recognition domain (CRD) as determined by searching for statistically significant matches in the hidden Markov model (HMM)-based PFAM and SMART databases of conserved protein families/domains. (See Table 3.) Data from MOTIFS and PROFILESCAN analyses, and BLAST analyses against the PRODOM and DOMO databases, provide further corroborative evidence that SEQ ID NO:18 is a carbohydrate-binding protein of the C-type lectin class.

As another example, SEQ ID NO:20 is 98% identical, from residue M1 to residue R110, to human regenerating protein I beta (GenBank ID g474308) as determined by the Basic Local Alignment Search Tool (BLAST).(See Table 2.) The BLAST probability score is 5.2E-55, which indicates the probability of obtaining the observed polypeptide sequence alignment by chance. SEQ ID NO:20 also has homology to Regenerating islet-derived 1 beta (a putative growth factor that may play a role in the regeneration of pancreatic islet cells, expressed only in the pancreas, the gene corresponding to which is overexpressed during colorectal carcinogenesis), as determined by BLAST analysis using the PROTEOME database. SEQ ID NO:20 also contains a C-type lectin (CTL) or carbohydrate-recognition domain as determined by searching for statistically significant matches in the hidden Markov model (HMM)-based SMART database of conserved protein families/domains. (See Table 3.) Data from BLIMPS analyses, and BLAST analyses against the PRODOM and DOMO databases, provide further corroborative evidence that SEQ ID NO:20 is a lectin.

SEQ ID NO:1, SEQ ID NO:3-7, SEQ ID NO:9, SEQ ID NO:11-17 and SEQ ID NO:19 were analyzed and annotated in a similar manner. The algorithms and parameters for the analysis of SEQ ID NO:1-20 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:21-40 or that distinguish between SEQ ID NO:21-40 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_{2—}YYYYY_N_{3—}N₄ represents a “stitched” sequence in which XXXXXX is the identification number of the cluster of sequences to which the algorithm was applied, and YYYYY is the number of the prediction generated by the algorithm, and N_{1,2,3 . . .} , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, GFG, | Exon prediction from genomic sequences using, for |
| ENST | example, GENSCAN (Stanford University, CA, USA) |
| | or FGENES (Computer Genomics Group, The Sanger |
| | Centre, Cambridge, UK). |
| GBI | Hand-edited analysis of genomic sequences. |
| FL | Stitched or stretched genomic sequences (see Example V). |
| INCY | Full length transcript and exon prediction from mapping of |
| | EST sequences to the genome. Genomic location and |
| | EST composition data are combined to predict the exons |
| | and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

Table 8 shows single nucleotide polymorphisms (SNPs) found in polynucleotide sequences of the invention, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification number for the EST in which the SNP was detected (EST ID), and column 4 shows the identification number for the SNP (SNP ID). Column 5 shows the position within the EST sequence at which the SNP is located (EST SNP), and column 6 shows the position of the SNP within the full-length polynucleotide sequence (CB1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses CHOP variants. Various embodiments of CHOP variants can have at least about 80%, at least about 90%, or at least about 95% amino acid sequence identity to the CHOP amino acid sequence, and can contain at least one functional or structural characteristic of CHOP.

Various embodiments also encompass polynucleotides which encode CHOP. In a particular embodiment, the invention encompasses a polynucleotide sequence comprising a sequence selected from the group consisting of SEQ ID NO:21-40, which encodes CHOP. The polynucleotide sequences of SEQ ID NO:21-40, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding CHOP. In particular, such a variant polynucleotide will have at least about 70%, or alternatively at least about 85%, or even at least about 95% polynucleotide sequence identity to a polynucleotide encoding CHOP. A particular aspect of the invention encompasses a variant of a polynucleotide comprising a sequence selected from the group consisting of SEQ ID NO:21-40 which has at least about 70%, or alternatively at least about 85%, or even at least about 95% polynucleotide sequence identity to a nucleic acid sequence selected from the group consisting of SEQ ID NO:21-40. Any one of the polynucleotide variants described above can encode a polypeptide which contains at least one functional or structural characteristic of CHOP.

In addition, or in the alternative, a polynucleotide variant of the invention is a splice variant of a polynucleotide encoding CHOP. A splice variant may have portions which have significant sequence identity to a polynucleotide encoding CHOP, but will generally have a greater or lesser number of nucleotides due to additions or deletions of blocks of sequence arising from alternate splicing during mRNA processing. A splice variant may have less than about 70%, or alternatively less than about 60%, or alternatively less than about 50% polynucleotide sequence identity to a polynucleotide encoding CHOP over its entire length; however, portions of the splice variant will have at least about 70%, or alternatively at least about 85%, or alternatively at least about 95%, or alternatively 100% polynucleotide sequence identity to portions of the polynucleotide encoding CHOP. For exarnple, a polynucleotide comprising a sequence of SEQ ID NO:23 and a polynucleotide comprising a sequence of SEQ ID NO:25 are splice variants of each other; a polynucleotide comprising a sequence of SEQ ID NO:27 and a polynucleotide comprising a sequence of SEQ ID NO:28 are splice variants of each other; and a polynucleotide comprising a sequence of SEQ ID NO:30, a polynucleotide comprising a sequence of SEQ ID NO:35, and a polynucleotide comprising a sequence of SEQ ID NO:36 are splice variants of each other. Any one of the splice variants described above can encode a polypeptide which contains at least one functional or structural characteristic of CHOP.

It will be appreciated by those skilled in the art that as a result of the degeneracy of the genetic code, a multitude of polynucleotide sequences encoding CHOP, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring CHOP, and all such variations are to be considered as being specifically disclosed.

Although polynucleotides which encode CHOP and its variants are generally capable of hybridizing to polynucleotides encoding naturally occurring CHOP under appropriately selected conditions of stringency, it may be advantageous to produce polynucleotides encoding CHOP or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding CHOP and its derivatives without altering the encoded amino acid sequences include the production of RNA transcripts having more desirable properties, such as a greater half-life, than transcripts produced from the naturally occurring sequence.

The invention also encompasses production of polynucleotides which encode CHOP and CHOP derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding CHOP or any fragment thereof.

Embodiments of the invention can also include polynucleotides that are capable of hybridizing to the claimed polynucleotides, and, in particular, to those having the sequences shown in SEQ ID NO:21-40 and fragments thereof, under various conditions of stringency (Wahl, G. M. and S. L. Berger (1987) Methods Enzymol. 152:399-407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley VCH, New York N.Y., pp. 856-853).

The nucleic acids encoding CHOP may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1:111-119). In this method, multiple restriction enzyme digestions and ligations may be used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (BD Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate programs to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode CHOP may be cloned in recombinant DNA molecules that direct expression of CHOP, or fragments or functional equivalents thereof, in appropriate host cells. Due to the inherent degeneracy of the genetic code, other polynucleotides which encode substantially the same or a functionally equivalent polypeptides may be produced and used to express CHOP.

The polynucleotides of the invention can be engineered using methods generally known in the art in order to alter CHOP-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat Biotechnol. 14:315-319) to alter or improve the biological properties of CHOP, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding CHOP may be synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, CHOP itself or a fragment thereof may be synthesized using chemical methods known in the art. For example, peptide synthesis can be performed using various solution-phase or solid-phase techniques (Creighton, T. (1984) Proteins, Structures and Molecular Properties, WH Freeman, New York N.Y., pp. 55-60; Roberge, J. Y. et al. (1995) Science 269:202-204). Automated synthesis may be achieved using the ABI 431A peptide synthesizer (Applied Biosystems). Additionally, the amino acid sequence of CHOP, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing (Creighton, supra, pp. 28-53).

In order to express a biologically active CHOP, the polynucleotides encoding CHOP or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding CHOP. Such elements may vary in their strength and specificity. Specific initiation signals may also be used to achieve more efficient translation of polynucleotides encoding CHOP. Such signals include the ATG initiation codon and adjacent sequences, e.g. the Kozak sequence. In cases where a polynucleotide sequence encoding CHOP and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding CHOP and appropriate transcriptional and translational control elements. These methods include in vitro recombinant DNA techniques, synthetic techniques, and in vivo genetic recombination (Sambrook and Russell, supra, ch. 1-4, and 8; Ausubel et al., supra, ch. 1, 3, and 15).

A variety of expression vector/host systems may be utilized to contain and express polynucleotides encoding CHOP. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook and Russell, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding CHOP. For example, routine cloning, subcloning, and propagation of polynucleotides encoding CHOP can be achieved using a multifunctional E. coli vector such as PBLUESCRIPT (Stratagene, La Jolla Calif.) or PSPORT1 plasmid (Invitrogen). Ligation of polynucleotides encoding CHOP into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of CHOP are needed, e.g. for the production of antibodies, vectors which direct high level expression of CHOP may be used. For example, vectors containing the strong, inducible SP6 or T7 bacteriophage promoter may be used.

Yeast expression systems may be used for production of CHOP. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Bio/Technology 12:181-184).

Plant systems may also be used for expression of CHOP. Transcription of polynucleotides encoding CHOP may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding CHOP may be ligated into an adenovirus transcription/translation complex consisting of the late promoter and tripartite leader sequence. Insertion in a non-essential E1 or E3 region of the viral genome may be used to obtain infective virus which expresses CHOP in host cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet. 15:345-355).

For long term production of recombinant proteins in mammalian systems, stable expression of CHOP in cell lines is preferred. For example, polynucleotides encoding CHOP can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., trpB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; BD Clontech), β-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be confirmed. For example, if the sequence encoding CHOP is inserted within a marker gene sequence, transformed cells containing polynucleotides encoding CHOP can be identified by the absence of marker gene function. Alternatively, a marker gene can be placed in tandem with a sequence encoding CHOP under the control of a single promoter. Expression of the marker gene in response to induction or selection usually indicates expression of the tandem gene as well.

In general, host cells that contain the polynucleotide encoding CHOP and that express CHOP may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of CHOP using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on CHOP is preferred, but a competitive binding assay may be employed. These and other assays are well known in the art (Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding CHOP include oligolabeling, nick translation, end-labeling, or PCR amplification using a labeled nucleotide. Alternatively, polynucleotides encoding CHOP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding CHOP may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode CHOP may be designed to contain signal sequences which direct secretion of CHOP through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding CHOP may be ligated to a heterologous sequence resulting in translation of a fusion protein in any of the aforementioned host systems. For example, a chimeric CHOP protein containing a heterologous moiety that can be recognized by a commercially available antibody may facilitate the screening of peptide libraries for inhibitors of CHOP activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the CHOP encoding sequence and the heterologous protein sequence, so that CHOP may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled CHOP may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

CHOP, fragments of CHOP, or variants of CHOP may be used to screen for compounds that specifically bind to CHOP. One or more test compounds may be screened for specific binding to CHOP. In various embodiments, 1, 2, 3, 4, 5, 10, 20, 50, 100, or 200 test compounds can be screened for specific binding to CHOP. Examples of test compounds can include antibodies, anticalins, oligonucleotides, proteins (e.g., ligands or receptors), or small molecules.

In related embodiments, variants of CHOP can be used to screen for binding of test compounds, such as antibodies, to CHOP, a variant of CHOP, or a combination of CHOP and/or one or more variants CHOP. In an embodiment, a variant of CHOP can be used to screen for compounds that bind to a variant of CHOP, but not to CHOP having the exact sequence of a sequence of SEQ ID NO:1-20. CHOP variants used to perform such screening can have a range of about 50% to about 99% sequence identity to CHOP, with various embodiments having 60%, 70%, 75%, 80%, 85%, 90%, and 95% sequence identity.

In an embodiment, a compound identified in a screen for specific binding to CHOP can be closely related to the natural ligand of CHOP, e.g., a ligand or fragment thereof, a natural substrate, a structural or functional mimetic, or a natural binding partner (Coligan, J. E. et al. (1991) Current Protocols in Immunology 1(2):Chapter 5). In another embodiment, the compound thus identified can be a natural ligand of a receptor CHOP (Howard, A. D. et al. (2001) Trends Pharmacol. Sci.22:132-140; Wise, A. et al. (2002) Drug Discovery Today 7:235-246).

In other embodiments, a compound identified in a screen for specific binding to CHOP can be closely related to the natural receptor to which CHOP binds, at least a fragment of the receptor, or a fragment of the receptor including all or a portion of the ligand binding site or binding pocket. For example, the compound may be a receptor for CHOP which is capable of propagating a signal, or a decoy receptor for CHOP which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Amgen Inc., Thousand Oaks Calif.), which is efficacious for treating rheumatoid arthritis in humans. Etanercept is an engineered p75 tumor necrosis factor (TNF) receptor dimer linked to the Fc portion of human IgG₁ (Taylor, P. C. et al. (2001) Curr. Opin. Immunol. 13:611-616).

In one embodiment, two or more antibodies having similar or, alternatively, different specificities can be screened for specific binding to CHOP, fragments of CHOP, or variants of CHOP. The binding specificity of the antibodies thus screened can thereby be selected to identify particular fragments or variants of CHOP. In one embodiment, an antibody can be selected such that its binding specificity allows for preferential identification of specific fragments or variants of CHOP. In another embodiment, an antibody can be selected such that its binding specificity allows for preferential diagnosis of a specific disease or condition having increased, decreased, or otherwise abnormal production of CHOP.

In an embodiment, anticalins can be screened for specific binding to CHOP, fragments of CHOP, or variants of CHOP. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit CHOP involves producing appropriate cells which express CHOP, either as a secreted protein or on the cell membrane. Preferred cells can include cells from mammals, yeast, Drosophila, or E. coli. Cells expressing CHOP or cell membrane fractions which contain CHOP are then contacted with a test compound and binding, stimulation, or inhibition of activity of either CHOP or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with CHOP, either in solution or affixed to a solid support, and detecting the binding of CHOP to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991) J. Biol. Chem. 266:10982-10988).

CHOP, fragments of CHOP, or variants of CHOP may be used to screen for compounds that modulate the activity of CHOP. Such compounds may include agonists, antagonists, or partial or inverse agonists. In one embodiment, an assay is performed under conditions permissive for CHOP activity, wherein CHOP is combined with at least one test compound, and the activity of CHOP in the presence of a test compound is compared with the activity of CHOP in the absence of the test compound. A change in the activity of CHOP in the presence of the test compound is indicative of a compound that modulates the activity of CHOP. Alternatively, a test compound is combined with an in vitro or cell-free system comprising CHOP under conditions suitable for CHOP activity, and the assay is performed. In either of these assays, a test compound which modulates the activity of CHOP may do so indirectly and need not come in direct contact with the test compound. At least one and up to a plurality of test compounds may be screened.

In another embodiment, polynucleotides encoding CHOP or their mammalian homologs may be “knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding CHOP may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding CHOP can also be used to create “knockin” humanized animals (pigs) or transgenic animals (nice or rats) to model human disease. With knockin technology, a region of a polynucleotide encoding CHOP is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress CHOP, e.g., by secreting CHOP in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of CHOP and carbohydrate-associated proteins. In addition, examples of tissues expressing CHOP can be found in Table 6 and can also be found in Example XI. Therefore, CHOP appears to play a role in carbohydrate metabolism, cell proliferative, autoimmune/inflammatory, reproductive, and neurological disorders. In the treatment of disorders associated with increased CHOP expression or activity, it is desirable to decrease the expression or activity of CHOP. In the treatment of disorders associated with decreased CHOP expression or activity, it is desirable to increase the expression or activity of CHOP.

Therefore, in one embodiment, CHOP or a fragment or derivative thereof may be administered to a subject to treat or prevent a disorder associated with decreased expression or activity of CHOP. Examples of such disorders include, but are not limited to, a carbohydrate metabolism disorder such as diabetes, insulin-dependent diabetes mellitus, non-insulin-dependent diabetes mellitus, hypoglycemia, glucagonoma, galactosemia, hereditary fructose intolerance, fructose-1,6-diphosphatase deficiency, obesity, congenital type II dyserythropoietic anemia, mannosidosis, neuraminidase deficiency, galactose epimerase deficiency, a glycogen storage disease, a lysosomal storage disease, fructosuria, pentosuria, a carbohydrate-deficient glycoprotein syndrome (CDGS types 1A and 1B), an autoimmune thyroid disorder, aspartylglycosaminuria, GM₁ gangliosidosis, GM₂ gangliosidosis, β-galactosidase deficiency, β-N-acetylhexosaminidase deficiency, a glycolipid storage disease, neurological dysfunction, sialidosis, hepatosplenomegaly, and an inherited abnormality of pyruvate metabolism; a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a reproductive disorder such as a disorder of prolactin production, infertility, including tubal disease, ovulatory defects, endometriosis, a disruption of the estrous cycle, a disruption of the menstrual cycle, polycystic ovary syndrome, ovarian hyperstimulation syndrome, an endometrial or ovarian tumor, a uterine fibroid, autoimmune disorders, ectopic pregnancy, teratogenesis; cancer of the breast, fibrocystic breast disease, galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, gynecomastia, hypergonadotropic and hypogonadotropic hypogonadism, pseudohermaphroditism, azoospermia, premature ovarian failure, acrosin deficiency, delayed puperty, retrograde ejaculation and anejaculation, haemangioblastomas, cystsphaeochromocytomas, paraganglioma, cystadenomas of the epididymis, and endolymphatic sac tumours; and a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia.

In another embodiment, a vector capable of expressing CHOP or a fragment or derivative thereof may be administered to a subject to treat or prevent a disorder associated with decreased expression or activity of CHOP including, but not limited to, those described above.

In a further embodiment, a composition comprising a substantially purified CHOP in conjunction with a suitable pharmaceutical carrier may be administered to a subject to treat or prevent a disorder associated with decreased expression or activity of CHOP including, but not limited to, those provided above.

In still another embodiment, an agonist which modulates the activity of CHOP may be administered to a subject to treat or prevent a disorder associated with decreased expression or activity of CHOP including, but not limited to, those listed above.

In a further embodiment, an antagonist of CHOP may be administered to a subject to treat or prevent a disorder associated with increased expression or activity of CHOP. Examples of such disorders include, but are not limited to, those carbohydrate metabolism, cell proliferative, autoimmune/inflammatory, reproductive, and neurological disorders described above. In one aspect, an antibody which specifically binds CHOP may be used directly as an antagonist or indirectly as a targeting or delivery mechanism for bringing a pharmaceutical agent to cells or tissues which express CHOP.

In an additional embodiment, a vector expressing the complement of the polynucleotide encoding CHOP may be administered to a subject to treat or prevent a disorder associated with increased expression or activity of CHOP including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of CHOP may be produced using methods which are generally known in the art. In particular, purified CHOP may be used to produce antibodies or to screen libraries of pharmaceutical agents to identify those which specifically bind CHOP. Antibodies to CHOP may also be generated using methods that are well known in the art. Such antibodies may include, but are not limited to, polyclonal, monoclonal, chimeric, and single chain antibodies, Fab fragments, and fragments produced by a Fab expression library. In an embodiment, neutralizing antibodies (i.e., those which inhibit dimer formation) can be used therapeutically. Single chain antibodies (e.g., from camels or llamas) may be potent enzyme inhibitors and may have application in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with CHOP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to CHOP have an amino acid sequence consisting of at least about 5 amino acids, and generally will consist of at least about 10 amino acids. It is also preferable that these oligopeptides, peptides, or fragments are substantially identical to a portion of the amino acid sequence of the natural protein. Short stretches of CHOP amino acids may be fused with those of another protein, such as KLH, and antibodies to the chimeric molecule may be produced.

Monoclonal antibodies to CHOP may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce CHOP-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for CHOP may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between CHOP and its specific antibody. A two-site, monoclonal-based immunoassay utilizing monoclonal antibodies reactive to two non-interfering CHOP epitopes is generally used, but a competitive binding assay may also be employed (Pound, supra).

Various methods such as Scatchard analysis in conjunction with radioimmunoassay techniques may be used to assess the affinity of antibodies for CHOP. Affinity is expressed as an association constant, Kₐ, which is defined as the molar concentration of CHOP-antibody complex divided by the molar concentrations of free antigen and free antibody under equilibrium conditions. The Kₐ determined for a preparation of polyclonal antibodies, which are heterogeneous in their affinities for multiple CHOP epitopes, represents the average affinity, or avidity, of the antibodies for CHOP. The Kₐ determined for a preparation of monoclonal antibodies, which are monospecific for a particular CHOP epitope, represents a true measure of affinity. High-affinity antibody preparations with Kₐ ranging from about 10⁹ to 10¹² L/mole are preferred for use in immunoassays in which the CHOP-antibody complex must withstand rigorous manipulations. Low-affinity antibody preparations with Kₐ ranging from about 10⁶ to 10⁷ L/mole are preferred for use in immunopurification and similar procedures which ultimately require dissociation of CHOP, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of CHOP-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding CHOP, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding CHOP. Such technology is well known in the art, and antisense oligonucleotides or larger fragments can be designed from various locations along the coding or control regions of sequences encoding CHOP (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469-475; Scanlon, K. J. et al. (1995) FASEB J. 9:1288-1296). Antisense sequences can also be introduced intracellularly through the use of viral vectors, such as retrovirus and adeno-associated virus vectors (Miller, A. D. (1990) Blood 76:271-278; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding CHOP may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in CHOP expression or regulation causes disease, the expression of CHOP from an appropriate population of transduced cells may alleviate the clinical manifestations caused by the genetic deficiency.

In a further embodiment of the invention, diseases or disorders caused by deficiencies in CHOP are treated by constructing mammalian expression vectors encoding CHOP and introducing these vectors by mechanical means into CHOP-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Récipon (1998) Curr. Opin. Biotechnol. 9:445-450).

Expression vectors that may be effective for the expression of CHOP include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (BD Clontech, Palo Alto Calif.). CHOP may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding CHOP from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to CHOP expression are treated by constructing a retrovirus vector consisting of (i) the polynucleotide encoding CHOP under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding CHOP to cells which have one or more genetic abnormalities with respect to the expression of CHOP. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999; Annu. Rev. Nutr. 19:511-544) and Verma, I. M. and N. Somia (1997; Nature 18:389:239-242).

In another embodiment, a herpes-based, gene therapy delivery system is used to deliver polynucleotides encoding CHOP to target cells which have one or more genetic abnormalities with respect to the expression of CHOP. The use of herpes simplex virus (HSV)-based vectors may be especially valuable for introducing CHOP to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding CHOP to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for CHOP into the alphavirus genome in place of the capsid-coding region results in the production of a large number of CHOP-coding RNAs and the synthesis of high levels of CHOP in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of CHOP into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding CHOP.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding CHOP. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytosine, guanine, thymine, and uracil which are not as easily recognized by endogenous endonucleases.

In other embodiments of the invention, the expression of one or more selected polynucleotides of the present invention can be altered, inhibited, decreased, or silenced using RNA interference (RNAi) or post-transcriptional gene silencing (PTGS) methods known in the art. RNAi is a post-transcriptional mode of gene silencing in which double-stranded RNA (dsRNA) introduced into a targeted cell specifically suppresses the expression of the homologous gene (i.e., the gene bearing the sequence complementary to the dsRNA). This effectively knocks out or substantially reduces the expression of the targeted gene. PTGS can also be accomplished by use of DNA or DNA fragments as well. RNAi methods are described by Fire, A. et al. (1998; Nature 391:806-811) and Gura, T. (2000; Nature 404:804-808). PTGS can also be initiated by introduction of a complementary segment of DNA into the selected tissue using gene delivery and/or viral vector delivery methods described herein or known in the art.

RNAi can be induced in mammalian cells by the use of small interfering RNA also known as siRNA. siRNA are shorter segments of dsRNA (typically about 21 to 23 nucleotides in length) that result in vivo from cleavage of introduced dsRNA by the action of an endogenous ribonuclease. siRNA appear to be the mediators of the RNAi effect in mammals. The most effective siRNAs appear to be 21 nucleotide dsRNAs with 2 nucleotide 3′ overhangs. The use of siRNA for inducing RNAi in mammalian cells is described by Elbashir, S. M. et al. (2001; Nature 411:494-498).

siRNA can be generated indirectly by introduction of dsRNA into the targeted cell. Alternatively, siRNA can be synthesized directly and introduced into a cell by transfection methods and agents described herein or known in the art (such as liposome-mediated transfection, viral vector methods, or other polynucleotide delivery/introductory methods). Suitable siRNAs can be selected by examining a transcript of the target polynucleotide (e.g., mRNA) for nucleotide sequences downstream from the AUG start codon and recording the occurrence of each nucleotide and the 3′ adjacent 19 to 23 nucleotides as potential siRNA target sites, with sequences having a 21 nucleotide length being preferred. Regions to be avoided for target siRNA sites include the 5′ and 3′ untranslated regions (UTRs) and regions near the start codon (within 75 bases), as these may be richer in regulatory protein binding sites. UTR-binding proteins and/or translation initiation complexes may interfere with binding of the siRNP endonuclease complex. The selected target sites for siRNA can then be compared to the appropriate genome database (e.g., human, etc.) using BLAST or other sequence comparison algorithms known in the art. Target sequences with significant homology to other coding sequences can be eliminated from consideration. The selected siRNAs can be produced by chemical synthesis methods known in the art or by in vitro transcription using commercially available methods and kits such as the SILENCER siRNA construction kit (Ambion, Austin Tex.).

In alternative embodiments, long-term gene silencing and/or RNAi effects can be induced in selected tissue using expression vectors that continuously express siRNA. This can be accomplished using expression vectors that are engineered to express hairpin RNAs (shRNAs) using methods known in the art (see, e.g., Brummelkamp, T. R. et al. (2002) Science 296:550-553; and Paddison, P. J. et al. (2002) Genes Dev. 16:948-958). In these and related embodiments, shRNAs can be delivered to target cells using expression vectors known in the art. An example of a suitable expression vector for delivery of siRNA is the PSILENCER1.0-U6 (circular) plasmid (Ambion). Once delivered to the target tissue, shRNAs are processed in vivo into siRNA-like molecules capable of carrying out gene-specific silencing.

In various embodiments, the expression levels of genes targeted by RNAi or PTGS methods can be determined by assays for mRNA and/or protein analysis. Expression levels of the mRNA of a targeted gene can be determined, for example, by northern analysis methods using the NORTHERNMAX-GLY kit (Ambion); by microarray methods; by PCR methods; by real time PCR methods; and by other RNA/polynucleotide assays known in the art or described herein. Expression levels of the protein encoded by the targeted gene can be determined, for example, by microarray methods; by polyacrylamide gel electrophoresis; and by Western analysis using standard techniques known in the art.

An additional embodiment of the invention encompasses a method for screening for a compound which is effective in altering expression of a polynucleotide encoding CHOP. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased CHOP expression or activity, a compound which specifically inhibits expression of the polynucleotide encoding CHOP may be therapeutically useful, and in the treatment of disorders associated with decreased CHOP expression or activity, a compound which specifically promotes expression of the polynucleotide encoding CHOP may be therapeutically useful.

In various embodiments, one or more test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding CHOP is exposed to at least one test compound thus obtained. The sample may comprise, for example, an intact or permeabilized cell, or an in vitro cell-free or reconstituted biochemical system. Alterations in the expression of a polynucleotide encoding CHOP are assayed by any method commonly known in the art. Typically, the expression of a specific nucleotide is detected by hybridization with a probe having a nucleotide sequence complementary to the sequence of the polynucleotide encoding CHOP. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1 999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of CHOP, antibodies to CHOP, and mimetics, agonists, antagonists, or inhibitors of CHOP.

In various embodiments, the compositions described herein, such as pharmaceutical compositions, may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery allows administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising CHOP or fragments thereof. For example, liposome preparations containing a cell-impermeable macromolecule may promote cell fusion and intracellular delivery of the macromolecule. Alternatively, CHOP or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example CHOP or fragments thereof, antibodies of CHOP, and agonists, antagonists or inhibitors of CHOP, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind CHOP may be used for the diagnosis of disorders characterized by expression of CHOP, or in assays to monitor patients being treated with CHOP or agonists, antagonists, or inhibitors of CHOP. Antibodies useful for diagnostic purposes may be prepared in the same manner as described above for therapeutics. Diagnostic assays for CHOP include methods which utilize the antibody and a label to detect CHOP in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring CHOP, including ELISAs, RIAs, and FACS, are known in the art and provide a basis for diagnosing altered or abnormal levels of CHOP expression. Normal or standard values for CHOP expression are established by combining body fluids or cell extracts taken from normal mammalian subjects, for example, human subjects, with antibodies to CHOP under conditions suitable for complex formation. The amount of standard complex formation may be quantitated by various methods, such as photometric means. Quantities of CHOP expressed in subject, control, and disease samples from biopsied tissues are compared with the standard values. Deviation between standard and subject values establishes the parameters for diagnosing disease.

In another embodiment of the invention, polynucleotides encoding CHOP may be used for diagnostic purposes. The polynucleotides which may be used include oligonucleotides, complementary RNA and DNA molecules, and PNAs. The polynucleotides may be used to detect and quantify gene expression in biopsied tissues in which expression of CHOP may be correlated with disease. The diagnostic assay may be used to determine absence, presence, and excess expression of CHOP, and to monitor regulation of CHOP levels during therapeutic intervention.

In one aspect, hybridization with PCR probes which are capable of detecting polynucleotides, including genomic sequences, encoding CHOP or closely related molecules may be used to identify nucleic acid sequences which encode CHOP. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding CHOP, allelic variants, or related sequences.

Probes may also be used for the detection of related sequences, and ray have at least 50% sequence identity to any of the CHOP encoding sequences. The hybridization probes of the subject invention nay be DNA or RNA and may be derived from the sequence of SEQ ID NO:21-40 or from genomic sequences including promoters, enhancers, and introns of the CHOP gene.

Means for producing specific hybridization probes for polynucleotides encoding CHOP include the cloning of polynucleotides encoding CHOP or CHOP derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²p or ³⁵S, or by enzymatic labels, such as alkaline phosphatase coupled to the probe via avidin/biotin coupling systems, and the like.

Polynucleotides encoding CHOP may be used for the diagnosis of disorders associated with expression of CHOP. Examples of such disorders include, but are not limited to, a carbohydrate metabolism disorder such as diabetes, insulin-dependent diabetes mellitus, non-insulin-dependent diabetes mellitus, hypoglycemia, glucagonoma, galactosemia, hereditary fructose intolerance, fructose-1,6-diphosphatase deficiency, obesity, congenital type II dyserythropoietic anemia, mannosidosis, neuraminidase deficiency, galactose epimerase deficiency, a glycogen storage disease, a lysosomal storage disease, fructosuria, pentosuria, a carbohydrate-deficient glycoprotein syndrome (CDGS types 1A and 1B), an autoimmune thyroid disorder, aspartylglycosaminuria, GM₁ gangliosidosis, GM₂ gangliosidosis, β-galactosidase deficiency, β-N-acetylhexosaminidase deficiency, a glycolipid storage disease, neurological dysfunction, sialidosis, hepatosplenomegaly, and an inherited abnormality of pyruvate metabolism; a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophlia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a reproductive disorder such as a disorder of prolactin production, infertility, including tubal disease, ovulatory defects, endometriosis, a disruption of the estrous cycle, a disruption of the menstrual cycle, polycystic ovary syndrome, ovarian hyperstimulation syndrome, an endometrial or ovarian tumor, a uterine fibroid, autoimmune disorders, ectopic pregnancy, teratogenesis; cancer of the breast, fibrocystic breast disease, galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, gynecomastia, hypergonadotropic and hypogonadotropic hypogonadism, pseudohermaphroditism, azoospermia, premature ovarian failure, acrosin deficiency, delayed puperty, retrograde ejaculation and anejaculation, haemangioblastomas, cystsphaeochromocytomas, paraganglioma, cystadenomas of the epididymis, and endolymphatic sac tumours; and a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia. Polynucleotides encoding CHOP may be used in Southern or northern analysis, dot blot, or other membrane-based technologies; in PCR technologies; in dipstick, pin, and multiformat ELISA-like assays; and in microarrays utilizing fluids or tissues from patients to detect altered CHOP expression. Such qualitative or quantitative methods are well known in the art.

In a particular embodiment, polynucleotides encoding CHOP may be used in assays that detect the presence of associated disorders, particularly those mentioned above. Polynucleotides complementary to sequences encoding CHOP may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding CHOP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of CHOP, a normal or standard profile for expression is established. This may be accomplished by combining body fluids or cell extracts taken from normal subjects, either animal or human, with a sequence, or a fragment thereof, encoding CHOP, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding CHOP may involve the use of PCR. These oligomers may be chemically synthesized, generated enzymatically, or produced in vitro. Oligomers will preferably contain a fragment of a polynucleotide encoding CHOP, or a fragment of a polynucleotide complementary to the polynucleotide encoding CHOP, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding CHOP may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding CHOP are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641).

Methods which may also be used to quantify the expression of CHOP include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem. 212:229-236). The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, CHOP, fragments of CHOP, or antibodies specific for CHOP may be used as elements on a microarray. The microarray ray be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for CHOP to quantify the levels of CHOP expression. In one embodiment, the antibodies are used as elements on a microarray, and protein expression levels are quantified by contacting the microarray with the sample and detecting the levels of protein bound to each array element (Lueking, A. et al. (1999) Anal. Biochem 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilharner (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/25116; Shalon, D. et al. (1995) PCT application WO95/35505; Heller, R. A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding CHOP may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding CHOP on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, CHOP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between CHOP and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with CHOP, or fragments thereof, and washed. Bound CHOP is then detected by methods well known in the art. Purified CHOP can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding CHOP specifically compete with a test compound for binding CHOP. In this manner, antibodies can be used to detect the presence of any peptide which shares one or more antigenic determinants with CHOP.

In additional embodiments, the nucleotide sequences which encode CHOP may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications, and publications mentioned above and below, including U.S. Ser. No. 60/425,423, U.S. Ser. No. 60/441,847, U.S. Ser. No. 60/453,882, U.S. Ser. No. 60/456,645, and U.S. Ser. No. 60/463,676, are hereby expressly incorporated by reference.

### EXAMPLES

I. Construction of cDNA Libraries

Incyte cDNAs are derived from cDNA libraries described in the LIFESEQ database (Incyte, Palo Alto Calif.). Some tissues are homogenized and lysed in guanidinium isothiocyanate, while others are homogenized and lysed in phenol or in a suitable mixture of denaturants, such as TRIZOL (Invitrogen), a monophasic solution of phenol and guanidine isothiocyanate. The resulting lysates are centrifuged over CsCl cushions or extracted with chloroform RNA is precipitated from the lysates with either isopropanol or sodium acetate and ethanol, or by other routine methods.

Phenol extraction and precipitation of RNA are repeated as necessary to increase RNA purity. In some cases, RNA is treated with DNase. For most libraries, poly(A)+RNA is isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA is isolated directly from tissue lysates using other RNA isolation kits, e.g., the POLY(A)PURE mRNA purification kit (Ambion, Austin Tex.).

In some cases, Stratagene is provided with RNA and constructs the corresponding cDNA libraries. Otherwise, cDNA is synthesized and cDNA libraries are constructed with the UNIZAP vector system (Stratagene) or SUPERSCRIPT plasmid system (Invitrogen), using the recommended procedures or similar methods known in the art (Ausubel et al., supra, ch. 5). Reverse transcription is initiated using oligo d(T) or random primers. Synthetic oligonucleotide adapters are ligated to double stranded cDNA, and the cDNA is digested with the appropriate restriction enzyme or enzymes. For most libraries, the cDNA is size-selected (300-1000 bp) using SEPHACRYL S1000, SEPHAROSE CL2B, or SEPHAROSE CL4B column chromatography (Amersham Biosciences) or preparative agarose gel electrophoresis. cDNAs are ligated into compatible restriction enzyme sites of the polylinker of a suitable plasmid, e.g., PBLUESCRIPT plasmid (Stratagene), PSPORT1 plasmid (Invitrogen, Carlsbad Calif.), PCDNA2.1 plasmid (Invitrogen), PBK-CMV plasmid (Stratagene), PCR2-TOPOTA plasmid (Invitrogen), PCMV-ICIS plasmid (Stratagene), pIGEN (Incyte, Palo Alto Calif.), pRARE (Incyte), or pINCY (Incyte), or derivatives thereof. Recombinant plasmids are transformed into competent E. coli cells including XL1-Blue, XL1-BlueMRF, or SOLR from Stratagene or DH5α, DH10B, or ElectroMAX DH10B from Invitrogen.

II. Isolation of cDNA Clones

Plasmids obtained as described in Example I are recovered from host cells by in vivo excision using the UNIZAP vector system (Stratagene) or by cell lysis. Plasmids are purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids are resuspended in 0.1 ml of distilled water and stored, with or without lyophilization, at 4° C.

Alternatively, plasmid DNA is amplified from host cell lysates using direct link PCR in a high-throughput format (Rao, V. B. (1994) Anal. Biochem. 216:1-14). Host cell lysis and thermal cycling steps are carried out in a single reaction mixture. Samples are processed and stored in 384-well plates, and the concentration of amplified plasmid DNA is quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II are sequenced as follows. Sequencing reactions are processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions are prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides are carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences are identified using standard methods (Ausubel et al., supra, ch. 7). Some of the cDNA sequences are selected for extension using the techniques disclosed in Example VIII.

Polynucleotide sequences derived from Incyte cDNAs are validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof are then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz, J. et al. (1998) Proc. Natl. Acad. Sci. USA 95:5857-5864; Letunic, l. et al. (2002) Nucleic Acids Res. 30:242-244). (HMM is a probabilistic approach which analyzes consensus primary structures of gene families; see, for example, Eddy, S. R. (1996) Curr. Opin. Struct. Biol. 6:361-365.) The queries are performed using programs based on BLAST, FASTA, BLIMPS, and HMMER. The Incyte cDNA sequences are assembled to produce full length polynucleotide sequences. Alternatively, GenBank cDNAs, GenBank ESTs, stitched sequences, stretched sequences, or Genscan-predicted coding sequences (see Examples IV and V) are used to extend Incyte cDNA assemblages to full length. Assembly is performed using programs based on Phred, Phrap, and Consed, and cDNA assemblages are screened for open reading frames using programs based on GeneMark, BLAST, and FASTA. The full length polynucleotide sequences are translated to derive the corresponding full length polypeptide sequences. Alternatively, a polypeptide may begin at any of the methionine residues of the full length translated polypeptide. Full length polypeptide sequences are subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (MiraiBio, Alameda Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences are also used to identify polynucleotide sequence fragments from SEQ ID NO:21-40. Fragments from about 20 to about 4000 nucleotides which are useful in hybridization and amplification technologies are described in Table 4, column 2.

IV. Identification and Editing of Coding Sequences from Genomic DNA

Putative carbohydrate-associated proteins are initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once is set to 30 kb. To determine which of these Genscan predicted cDNA sequences encode carbohydrate-associated proteins, the encoded polypeptides are analyzed by querying against PFAM models for carbohydrate-associated proteins. Potential carbohydrate-associated proteins are also identified by homology to Incyte cDNA sequences that have been annotated as carbohydrate-associated proteins. These selected Genscan-predicted sequences are then compared by BLAST analysis to the genpept and gbpri public databases. Where necessary, the Genscan-predicted sequences are then edited by comparison to the top BLAST hit from genpept to correct errors in the sequence predicted by Genscan, such as extra or omitted exons. BLAST analysis is also used to find any Incyte cDNA or public cDNA coverage of the Genscan-predicted sequences, thus providing evidence for transcription. When Incyte cDNA coverage is available, this information is used to correct or confirm the Genscan predicted sequence. Full length polynucleotide sequences are obtained by assembling Genscan-predicted coding sequences with Incyte cDNA sequences and/or public cDNA sequences using the assembly process described in Example III. Alternatively, full length polynucleotide sequences are derived entirely from edited or unedited Genscan-predicted coding sequences.

V. Assembly of Genomic Sequence Data with cDNA Sequence Data

“Stitched” Sequences

Partial cDNA sequences are extended with exons predicted by the Genscan gene identification program described in Example IV. Partial cDNAs assembled as described in Example III are mapped to genomic DNA and parsed into clusters containing related cDNAs and Genscan exon predictions from one or more genomic sequences. Each cluster is analyzed using an algorithm based on graph theory and dynamic programming to integrate cDNA and genomic information, generating possible splice variants that are subsequently confirmed, edited, or extended to create a full length sequence. Sequence intervals in which the entire length of the interval is present on more than one sequence in the cluster are identified, and intervals thus identified are considered to be equivalent by transitivity. For example, if an interval is present on a cDNA and two genomic sequences, then all three intervals are considered to be equivalent This process allows unrelated but consecutive genomic sequences to be brought together, bridged by cDNA sequence. Intervals thus identified are then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) are given preference over linkages which change parent type (cDNA to genomic sequence). The resultant stitched sequences are translated and compared by BLAST analysis to the genpept and gbpri public databases. Incorrect exons predicted by Genscan are corrected by comparison to the top BLAST hit from genpept. Sequences are further extended with additional cDNA sequences, or by inspection of genomic DNA, when necessary.

“Stretched” Sequences

Partial DNA sequences are extended to full length with an algorithm based on BLAST analysis. First, partial cDNAs assembled as described in Example III are queried against public databases such as the GenBank primate, rodent, mammalian, vertebrate, and eukaryote databases using the BLAST program. The nearest GenBank protein homolog is then compared by BLAST analysis to either Incyte cDNA sequences or GenScan exon predicted sequences described in Example IV. A chimeric protein is generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both are used as probes to search for homologous genomic sequences from the public human genome databases. Partial DNA sequences are therefore “stretched” or extended by the addition of homologous genomic sequences. The resultant stretched sequences are examined to determine whether they contain a complete gene.

VI. Chromosomal Mapping of CHOP Encoding Polynucleotides

The sequences used to assemble SEQ ID NO:21-40 are compared with sequences from the Incyte LIFESEQ database and public domain databases using BLAST and other implementations of the Smith-Waterman algorithm. Sequences from these databases that matched SEQ ID NO:21-40 are assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon are used to determine if any of the clustered sequences have been previously mapped. Inclusion of a mapped sequence in a cluster results in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook and Russell, supra, ch. 7; Ausubel et al., supra, ch. 4).

Analogous computer techniques applying BLAST are used to search for identical or related molecules in databases such as GenBank or LIFESEQ (Incyte). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding CHOP are analyzed with respect to the tissue sources from which they are derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding CHOP. cDNA sequences and cDNA library/tissue information are found in the LIFESEQ database (Incyte, Palo Alto Calif.).

VIII. Extension of CHOP Encoding Polynucleotides

Full length polynucleotides are produced by extension of an appropriate fragment of the full length molecule using oligonucleotide primers designed from this fragment. One primer is synthesized to initiate 5′ extension of the known fragment, and the other primer is synthesized to initiate 3′ extension of the known fragment. The initial primers are designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations is avoided.

Selected human cDNA libraries are used to extend the sequence. If more than one extension is necessary or desired, additional or nested sets of primers are designed.

High fidelity amplification is obtained by PCR using methods well known in the art. PCR is performed in 96-well plates using the PTC-200 thermal cycler (MJ Research, Inc.). The reaction mix contains DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ are as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well is determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimneter plate (Corning Costar, Acton Mass.), allowing the DNA to bind to the reagent. The plate is scanned in a Fluoroskan II (Labsystems Oy, Helsinki, Finland) to measure the fluorescence of the sample and to quantify the concentration of DNA. A 5 μl to 10 μl aliquot of the reaction mixture is analyzed by electrophoresis on a 1% agarose gel to determine which reactions are successful in extending the sequence.

The extended nucleotides are desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides are separated on low concentration (0.6 to 0.8%) agarose gels, fragments are excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells are selected on antibiotic-containing media, and individual colonies are picked and cultured overnight at 37° C. in 384-well plates in LB/2x carb liquid media.

The cells are lysed, and DNA is amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA is quantified by PICOGREEN reagent (Molecular Probes) as described above. Samples with low DNA recoveries are reamplified using the same conditions as described above. Samples are diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in CHOP Encoding Polynucleotides

Common DNA sequence variants known as single nucleotide polymorphisms (SNPs) are identified in SEQ ID NO:21-40 using the LIFESEQ database (Incyte). Sequences from the same gene are clustered together and assembled as described in Example III, allowing the identification of all sequence variants in the gene. An algorithm consisting of a series of filters is used to distinguish SNPs from other sequence variants. Preliminary filters remove the majority of baseball errors by requiring a minimum Phred quality score of 15, and remove sequence alignment errors and errors resulting from improper trimming of vector sequences, chimeras, and splice variants. An automated procedure of advanced chromosome analysis is applied to the original chromatogram files in the vicinity of the putative SNP. Clone error filters use statistically generated algorithms to identify errors introduced during laboratory processing, such as those caused by reverse transcriptase, polymerase, or somatic mutation. Clustering error filters use statistically generated algorithms to identify errors resulting from clustering of close homologs or pseudogenes, or due to contamination by non-human sequences. A final set of filters removes duplicates and SNPs found in immunoglobulins or T-cell receptors.

Certain SNPs are selected for further characterization by mass spectrometry using the high throughput MASSARRAY system (Sequenom, Inc.) to analyze allele frequencies at the SNP sites in four different human populations. The Caucasian population comprises 92 individuals (46 male, 46 female), including 83 from Utah, four French, three Venezualan, and two Amish individuals. The African population comprises 194 individuals (97 male, 97 female), all African Americans. The Hispanic population comprises 324 individuals (162 male, 162 female), all Mexican Hispanic. The Asian population comprises 126 individuals (64 male, 62 female) with a reported parental breakdown of 43% Chinese, 31% Japanese, 13% Korean, 5% Vietnamese, and 8% other Asian. Allele frequencies are first analyzed in the Caucasian population; in some cases those SNPs which show no allelic variance in this population are not further tested in the other three populations.

X. Labeling and Use of Individual Hybridization Probes

Hybridization probes derived from SEQ ID NO:21-40 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to NYTRAN PLUS nylon membranes (Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21mer), 1× first strand buffer, 0.03 units/μl RNase inhibitor, 500 μM dGTP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (BD Clontech, Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma-Aldrich, St. Louis Mo.) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X—Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte). Array elements that exhibit at least about a two-fold change in expression, a signal-to-background ratio of at least about 2.5, and an element spot size of at least about 40%, are considered to be differentially expressed.

Expression

In one example, SEQ ID NO:22 showed differential expression in C3A cells treated with a variety of steroids including progesterone, beclomethasone, medroxyprogesterone, budesonide, prednisone, dexamethasone, and betamethasone, versus untreated C3A cells, as determined by microarray analysis. Early confluent C3A cells were treated with either progesterone, beclomethasone, medroxyprogesterone, budesonide, prednisone, dexamethasone, or betamethasone (1, 10, and 100 μM) for 1, 3, and 6 hours. The treated cells were compared to untreated early confluent C3A cells. Relative to expression levels in the untreated C3A cells, expression of SEQ ID NO:22 was increased at least 2-fold in C3A cells treated with the following steroids, doses, and time points: progesterone, 1 μM for 3 or 6 hours, 10 μM for 1 or 3 hours, and 100 μM for 1 or 6 hours; beclomethasone, 1 μM for 1 hour, 10 μM for 1, 3, or 6 hours, and 100 μM for 3 hours; medroxyprogesterone, 1 μM for 1 hour, 10 μM for 1, 3, or 6 hours, and 100 μM for 3 hours; budesonide, 10 μM or 100 μM for 1, 3, or 6 hours; prednisone, 1 μM for 1 or 3 hours; dexamethasone, 1 μM for 1, 3, or 6 hours, and 10 μM for 1 hour; and betamethasone, 10 μM for 6 hours, and 100 μM for 1 or 3 hours. Therefore, in various embodiments, SEQ ID NO:22 can be used for one or more of the following: i) monitoring treatment of liver, endocrine, and reproductive diseases, ii) diagnostic assays for liver toxicity and clearance, and liver, endocrine, and reproductive diseases, and iii) developing therapeutics and/or other treatments for liver, endocrine, and reproductive diseases.

In another example, SEQ ID NO:22, SEQ ID NO:23, SEQ ID NO:24, and SEQ ID NO:25 showed tissue-specific expression as determined by microarray analysis. RNA samples isolated from a variety of normal human tissues were compared to a common reference sample. Tissues contributing to the reference sample were selected for their ability to provide a complete distribution of RNA in the human body and include brain (4%), heart (7%), kidney (3%), lung (8%), placenta (46%), small intestine (9%), spleen (3%), stomach (6%), testis (9%), and uterus (5%). The normal tissues assayed were obtained from at least three different donors. RNA from each donor was separately isolated and individually hybridized to the microarray. Since these hybridization experiments were conducted using a common reference sample, differential expression values are directly comparable from one tissue to another. The expression of SEQ ID NO:22 was increased by at least two-fold in pancreas and spleen samples, as compared to the reference sample. Therefore, SEQ ID NO:22 can be used as a tissue marker for pancreas and spleen. In addition, the expression of SEQ ID NO:23 was increased by at least two-fold in liver samples, as compared to the reference sample. Therefore, SEQ ID NO:23 can be used as a tissue marker for liver. In addition, the expression of SEQ ID NO:24 was increased by at least two-fold in gallbladder samples, and at least 14-fold in liver samples. Therefore, SEQ ID NO:24 can be used as a tissue marker for gallbladder and liver. In addition, the expression of SEQ ID NO:25 was increased by at least two-fold in liver samples, as compared to the reference sample. Therefore, SEQ ID NO:25 can be used as a tissue marker for liver.

For example, expression of SEQ ID NO:31 showed differential expression and was up-regulated in a malignant versus a nonmalignant mammary epithelial cell line as determined by microarray analysis. The gene expression profile of a nonmalignant mammary epithelial cell line was compared to the gene expression profiles of breast carcinoma lines at different stages of tumor progression. Cell lines compared included: a) MCF-10A, a breast mammary gland cell line isolated from a female with fibrocystic breast disease; b) MCF-7, a nonmalignant breast adenocarcinoma cell line isolated from the pleural effusion of a female; c) T-47D, a breast carcinoma cell line isolated from a pleural effusion obtained from a female with an infiltrating ductal carcinoma of the breast; d) Sk-BR-3, a breast adenocarcinoma cell line isolated from a malignant pleural effusion of a female; e) BT-20, a breast carcinoma cell line derived in vitro from tumor mass isolated from a female; f) MDA-mb-435S, a spindle shaped strain that evolved from the parent line (435) isolated from the pleural effusion of a female with metastatic, ductal adenocarcinoma of the breast; and g) MDA-mb-231, a breast tumor cell line isolated from the pleural effusion of a female.

Expression of SEQ ID NO:31 was increased at least two-fold in one out of six malignant mammary epithelial cell lines investigated, the MDA-mb-435S cell line, as compared to controls. Therefore, in various embodiments, SEQ ID NO:31 can be used for one or more of the following: i) monitoring treatment of breast cancer, ii) diagnostic assays for breast cancer, and iii) developing therapeutics and/or other treatments for breast cancer.

In another experiment, expression of SEQ ID NO:31 showed differential expression and was up-regulated in human lung tumor tissue versus normal, uninvolved lung tissue from the same donor, as determined by microarray analysis.

Expression of SEQ ID NO:31 was increased at least two-fold in lung squamous cell carcinoma tissue as compared to uninvolved tissue from the lung of the same donor. Therefore, in various embodiments, SEQ ID NO:31 can be used for one or more of the following: i) monitoring treatment of lung cancer, ii) diagnostic assays for lung cancer, and iii) developing therapeutics and/or other treatments for lung cancer.

For example, SEQ ID NO:37 showed differential expression in PBMCs, as determined by microarray analysis. In one experiment, PBMCs were collected from the blood of 6 healthy volunteer donors using standard gradient separation. The PBMCs from each donor were placed in culture for 2 hours in the presence or absence of 10 ng/ml recombinant IL-4. IL-4-treated PBMCs and untreated control PBMCs from the different donors were pooled according to their respective treatments. The expression of SEQ ID NO:37 increased at least 2-fold in the IL-4-treated PBMCs, in comparison to the expression levels detected in the untreated cells. In another experiment, PBMCs from the blood of 6 healthy volunteer donors were isolated as described above. The PBMCs from each donor were placed in culture for 2 and 4 hours in the presence of one or more cytokines selected from one of the two following cytokine groups: a) cytokines associated positively with the inflammatory response (“pro-inflammatory”) such as IL-1β, IL-2, IL-6, IL-8, IL-12, IL-18, IFN-γ, and TNF-α; or b) cytokines negatively or neutrally associated with the inflammatory response (“anti-inflammatory”) such as IL-3, IL-4, IL-5, IL-7, IL-10, G-CSF, GM-CSF, leptin, LIF, and TGF-β. Cytokine-treated PBMCs and untreated control PBMCs from the different donors were pooled according to their respective treatments. The expression of SEQ ID NO:37 was increased at least 2-fold in the PBMCs treated with the “anti-inflammatory” group of cytokines, when compared to untreated PBMCs. In another experiment, PBMCs were collected from the blood of 5 healthy volunteer donors as described above. PBMCs from each donor were placed in culture for 2 and 4 hours in the presence of anti-inflammatory cytokines such as IL-3, IL-4, IL-5, IL-7, IL-10, G-CSF, GM-CSF, lectin, LIF, and TGF-β. Cytokine-treated PBMCs and untreated control PBMCs from the different donors were pooled according to their respective treatments. The expression of SEQ ID NO:37 was increased at least 2-fold in the PBMCs treated with anti-inflammatory cytokines for 2 or 4 hours, when compared to the levels of SEQ ID NO:37 detected in the untreated cells. Therefore, in various embodiments, SEQ ID NO:37 can be used for one or more of the following: i) monitoring treatment of inflammatory or immune disorders and related diseases and conditions, ii) diagnostic assays for inflammatory or immune disorders and related diseases and conditions, and iii) developing therapeutics and/or other treatments for inflammatory or immune disorders and related diseases and conditions.

In another example, SEQ ID NO:38 was differentially expressed in lung tumor tissue, as determined by microarray analysis. Six matched sets of lung adenocarcinoma or squamous cell carcinoma tissue were used, and gene expression levels compared to normal, grossly uninvolved lung tissue from the same donor (Roy Castle International Centre for Lung Cancer Research, Liverpool, UK). In all six tumor tissue samples examined, the expression level of SEQ ID NO:38 was increased in the tumor tissue, in comparison to the gene expression levels detected in the normal lung tissue. The expression ranged from at least 3.5-fold to at least 8.5-fold increased in the tumor tissue. Therefore, in various embodiments, SEQ ID NO:38 can be used for one or more of the following: i) monitoring treatment of lung cancer, ii) diagnostic assays for lung cancer, and iii) developing therapeutics and/or other treatments for lung cancer.

XII. Complementary Polynucleotides

Sequences complementary to the CHOP-encoding sequences, or any parts thereof, are used to detect, decrease, or inhibit expression of naturally occurring CHOP. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of CHOP. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the CHOP-encoding transcript.

XIII. Expression of CHOP

Expression and purification of CHOP is achieved using bacterial or virus-based expression systems. For expression of CHOP in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express CHOP upon induction with isopropyl beta-D-thiogalactopyranoside (IPTG). Expression of CHOP in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding CHOP by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. Natl. Acad. Sci. USA 91:3224-3227; Sandig, V. et al. (1996) Hum. Gene Ther. 7:1937-1945).

In most expression systems, CHOP is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from CHOP at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified CHOP obtained by these methods can be used directly in the assays shown in Examples XVII and XVIII, where applicable.

XIV. Functional Assays

CHOP function is assessed by expressing the sequences encoding CHOP at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; BD Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994; Flow Cytometry, Oxford, New York N.Y.).

The influence of CHOP on gene expression can be assessed using highly purified populations of cells transfected with sequences encoding CHOP and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding CHOP and other genes of interest can be analyzed by northern analysis or microarray techniques.

XV. Production of CHOP Specific Antibodies

CHOP substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the CHOP amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity (Ausubel et al., supra). Rabbits are immunized with the oligopeptide-KLH complex in complete Freund's adjuvant. Resulting antisera are tested for antipeptide and anti-CHOP activity by, for example, binding the peptide or CHOP to a substrate, blocking with 1% BSA, reacting with rabbit antisera, washing, and reacting with radio-iodinated goat anti-rabbit IgG.

XVI. Purification of Naturally Occurring CHOP Using Specific Antibodies

Naturally occurring or recombinant CHOP is substantially purified by immunoaffinity chromatography using antibodies specific for CHOP. An immunoaffinity column is constructed by covalently coupling anti-CHOP antibody to an activated chromatographic resin, such as CNBr-activated SEPHAROSE (Amersham Biosciences). After the coupling, the resin is blocked and washed according to the manufacturer's instructions.

Media containing CHOP are passed over the immunoaffinity column, and the column is washed under conditions that allow the preferential absorbance of CHOP (e.g., high ionic strength buffers in the presence of detergent). The column is eluted under conditions that disrupt antibody/CHOP binding (e.g., a buffer of pH 2 to pH 3, or a high concentration of a chaotrope, such as urea or thiocyanate ion), and CHOP is collected.

XVII. Identification of Molecules Which Interact with CHOP

CHOP, or biologically active fragments thereof, are labeled with ¹²⁵I Bolton-Hunter reagent (Bolton, A. E. and W. M. Hunter (1973) Biochem. J. 133:529-539). Candidate molecules previously arrayed in the wells of a multi-well plate are incubated with the labeled CHOP, washed, and any wells with labeled CHOP complex are assayed. Data obtained using different concentrations of CHOP are used to calculate values for the number, affinity, and association of CHOP with the candidate molecules.

Alternatively, molecules interacting with CHOP are analyzed using the yeast two-hybrid system as described in Fields, S. and O. Song (1989; Nature 340:245-246), or using commercially available kits based on the two-hybrid system, such as the MATCHMAKER system (BD Clontech).

CHOP may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of CHOP Activity

Galactosyltransferase Activity Assay #1

β1,3-galactosyltransferase and β1,4-galactosyltransferase activity of CHOP is determined by measuring the transfer of galactose from UDP-galactose to a GlcNAc-terminated oligosaccharide chain in a radioactive assay (Kolbinger, F. et al. (1998) J. Biol. Chem. 273:58-65; Hennet, T. et al. (1998) J. Biol. Chem. 273:58-65). An aliquot of CHOP is incubated with 14 μl of assay stock solution (180 mM sodium cacodylate, pH 6.5, 1 mg/ml bovine serum albumin, 0.26 mM UDP-galactose, 2 μl of UDP-[³H]galactose, 1 μl of MnCl₂ (500 MM), and 2.5 μl of GlcNAcβO—(CH₂)₈—CO₂Me (37 mg/ml in dimethyl sulfoxide)) for approximately 1 hr at 37° C. The reaction is quenched by the addition of 1 ml of water and loaded on a C18 Sep-Pak cartridge (Waters). The column is washed twice with 5 ml of water to remove unreacted UDP-[³H]galactose. The reaction product, [³H]galactosylated GlcNAcβO—(CH₂)₈—CO₂Me, remains bound to the column during the water washes and is eluted with 5 ml of methanol. The level of radioactivity in the eluted material is measured by liquid scintillation counting and is proportional to CHOP galactosyltransferase activity in the sample.

Galactosyltransferase Activity Assay #2

In the alternative, β1,4-galactosyltransferase activity of CHOP is determined by quantitating the transfer of [¹⁴C]galactose from UDP-[¹⁴C]Gal to ovalbumin. The approximately 50 μl reaction contains 50 mM HEPES (pH 7.35), 10 mM MnCl₂, 1.5 mg of ovalbumin, 50 mM NaCl, 5 μl UDP-[¹⁴C]galactose (25 nCi), and 5 μl of CHOP. The assay is incubated at 60° C. for 30 minutes terminated by the addition of ice-cold 2.5% phosphotungstic acid (w/v) in 1 M HCl. Unincorporated UDP-[¹⁴C]Gal is separated by filtration through Whatman GF/C glass fiber filters. The filters are washed once with 2.5% phosphotungstic acid (w/v) and then rinsed with ice-cold ethanol. The filters are dried, and radioactivity is determined using a scintillation counter. The amount of radioactivity is proportional to the activity of CHOP (Verdon, B. and Berger, E. (1983) in Galactosyltransferase. Methods of Enzymatic Analysis, Bergmeyer, H. et al. (eds.), Vol. III, 3rd Ed., Verlag Chemie, Weinheim, Deerfield Beach, Basel, pp. 374-381; Tilo, S. et al. (1996) J. Biol. Chem. 271:3398-3405).

Sialyltransferase Activity Assay #1

In the alternative, sialyltransferase activity of CHOP is assayed as follows. The sialyltransferase acceptors used in the assay are derived from aminophenylglycosides reacted with 6(5-fluorescein-carboxamido)-hexanoic acid succimidyl ester (FCHASE). Briefly, 10 mg p-aminophenylglycoside is dissolved in 0.5 ml of 0.2 M triethylamine acetate buffer, pH 8.2. FCHASE was dissolved in 0.5 ml of methanol and added to the aminophenylglycoside solution. The mixture is stirred in the dark for about 3 hr at room temperature, lyophilized, resuspended in approximately 200 μl of 50% acetonitrile, and spotted on a silica thin-layer chromatography (TLC) plate which is developed with an ethyl acetate/methanol/water/acetic acid solvent system. Following air drying in a fume hood, the yellow product is scraped, eluted with distilled water, concentrated, desalted, and bound to a Sep-Pak C18reverse phase cartridge. After washing the cartridge with several volumes of water, the product is eluted in 50% acetonitrile and quantitated by well-known spectrophotometric methods. Following preparation of the sialyltransferase acceptors, enzyme reactions are performed at 37° C. in 20 μl volumes in a buffer consisting of 50 mM MES (pH 6.0), 10 mM MnCl₂, with 0.2 or 1.0 mM labeled acceptor, 0.2 mM CMP-Neu5Ac donor, and various amounts of CHOP. The reaction is terminated by diluting the reaction with 10 mM NaOH prior to analysis by capillary electrophoresis. Capillary electrophoresis (CE) is performed using an Argon-ion laser-induced fluorescence detector (excitation=488 nm; emission=520 nm). The product peak, consisting of FCHASE-2,3-sialyl-N-acetyllactosamine, is identified and quantitated and is proportional to the sialyltransferase activity in the sample (Wakarchuk, W. et al. (1996) J. Biol. Chem. 271:19166-19173; Gilbert, M. (1996)J. Biol. Chem. 271:28271-28276).

Sialyltransferase Activity Assay #2

Alternatively, sialyltransferase activity of CHOP is assayed as follows. Sialyltransferase assays are performed in a reaction mixture containing 10 mM MgCl₂, 0.3% Triton CF-54, 100 mM sodium cacodylate buffer (pH 6.0), 0.66 mM4 unlabeled CMP-Neu5Ac donor, 4,400 dpm/μl CMP-[¹⁴C]Neu5Ac (tracer), the CHOP solution, and substrates in a total volume of 20 to 50 μl. The reaction mixture is incubated at 37° C. for 2 hr, then terminated by addition of 500 μl of water. The products are isolated by C18 Sep-Pak cartridge and analyzed by thin layer chromatography (TLC) using the solvent system ethanol/pyridine/n-butanol/acetate/water (100:10:10:3:30) (Okajima, T. et al. (1999) J. Biol. Chem 274:11479-11486). The radiolabeled products are visualized by standard autoradiography techniques familiar to persons skilled in the art and sialyltransferase activity of CHOP is proportional to the signal on the autoradiogram.

Sialyltransferase Activity Assay #3

In the alternative, sialyltransferase activity of CHOP is assayed as follows. Human embryonic kidney cells (293) are stably transfected with a plasmid encoding CHOP. The cells are grown to confluence in 225 cm² tissue culture flasks and harvested by scraping cells into phosphate-buffered saline (PBS). Cells are pelleted and resuspended in approximately 1 ml of 1% Triton X-100, 50 mM NaCl, 5 mM MnCl, and 25 mM MES (pH 6.0). The cell pellet is solubilized by repeated pipetting and vortexing. This crude homogenate is cleared by centrifugation at 1000×g for 10 min and used directly as the enzyme source. The assay mixture consists of 50 μM CMP-Neu5Ac with 250,000 cpm of CMP-[¹⁴C]Neu5Ac added as a tracer, 0.1% Triton CF-54, 20 mM cacodylate (pH 6.0) and 10 μl of CHOP-containing extract in a 30-μl reaction volume. Glycoprotein and glycolipid products are separated from CMP-Neu5Ac by gel filtration and the amount of label in the eluted fractions are quantitated to determine the relative amount of CHOP activity in the sample (Sjoberg, E. et al. (1996) J. Biol. Chem. 271:7450-7459).

O-Glucosyltransferase Transferase Activity Assay

O-glucosyltransferase activity of CHOP is assayed as follows. CHOP preparations are diluted with cold desalt buffer (20 mM Tris pH 8.0, 20% glycerol, 0.02% NaN₃) immediately prior to use. Peptide substrates (Kreppel, L. et al. (1999) J. Biol. Chem. 274:32015-32022) are used as acceptors at a concentration of approximately 3 mM. The peptides in the reaction are separated from the reactants using a SP-Sephadex column. The modified and unmodified peptides are loaded onto a Sep-pak C18 cartridge. Unmodified peptides are eluted with 50 mM formic acid, 10 ml of 50 mM formic acid containing 0.5 M NaCl, and 10 ml of distilled H₂O. Modified peptides are eluted from the cartridge directly into scintillation vials using methanol. Enzyme activity is expressed in terms of micromoles of GlcNAc transferred per minute, which is proportional to the level of CHOP activity in the sample (Kreppel et al., supra).

Mannosidase Activity Assay

Mannosidase activity in CHOP is measured by its ability to release mannose from Man₉ (GlcNAc)₂ oligosaccharide (Schweden, J. et al. (1986) Eur. J. Biochem. 157:563-570). CHOP, in 200 mM phosphate buffer, pH 6.5 and 1% Triton X-100, is mixed with [¹⁴C](Man₉)(GlcNAc)₂(2-3×10³ cpm) in a final volume of 30 μl at 37° C. for 60 minutes. The reaction is terminated by the addition of 30 μl glacial acetic acid. The amount of liberated [¹⁴C]mannose, analyzed by paper chromatography in 2-propanol/acetic acid/water (29/4/9, by volume), is proportional to the activity of CHOP in the starting sample.

Carbohydrate Binding Assay

CHOP activity is also demonstrated by the ability of CHOP to bind to β-galactoside sugars. CHOP is applied to a lactosyl-Sepharose column, and the column is eluted with 0.1 M lactose. The presence of CHOP in the eluate is detected by sodium dodecyl sulfate polyacrylamide gel electrophoresis and indicates the ability of CHOP to bind β-galactoside sugars.

Hyaluronan Hydrolysis Assay

CHOP activity is also measurable by its ability to hydrolyze hyaluronan (HA) (Lepperdinger, supra). Radioactively labeled HA is immobilized on microtiter plates with the aid of 1-ethyl-3-(3-dimethylaminopropyl)carbodiimide and N-hydroxy-sulfosuccinimide. The radioactivity solubilized after incubation with CHOP is measured using a liquid scintillation counter and is proportional to the activity of CHOP in the starting sample.

Cellular Transformation Assay

Alternatively, CHOP activity is measured by its ability to regulate transformation of NIH3T3 mouse fibroblast cells. A cDNA encoding CHOP is subcloned into an appropriate eukaryotic expression vector. This construct is transfected into NIH3T3 cells using methods known in the art. Transfected cells are compared with non-transfected cells for the following quantifiable properties characteristic of oncogenically transformed cells: growth in culture to high density associated with loss of contact inhibition, growth in suspension or in soft agar, lowered serum requirements, and ability to induce tumors when injected into immunodeficient mice. The activity of CHOP is proportional to the extent of transformation of NIH3T3 cells transfected with CHOP and non-transfected cells.

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | | Incyte | | Incyte | |
|---|---|---|---|---|---|
| Incyte | Polypeptide | Polypeptide | Polynucleotide | Polynucleotide | |
| Project ID | SEQ ID NO: | ID | SEQ ID NO: | ID | Incyte Full Length Clones |
| 7521032 | 1 | 7521032CD1 | 21 | 7521032CB1 | 95120941CA2 |
| 2936048 | 2 | 2936048CD1 | 22 | 2936048CB1 | |
| 7521726 | 3 | 7521726CD1 | 23 | 7521726CB1 | |
| 7523383 | 4 | 7523383CD1 | 24 | 7523383CB1 | 90072281CA2, 90072289CA2, 90073712CA2, 95145501CA2, |
| | | | | | 95170718CA2, 95170758CA2, 95170810CA2 |
| 7522027 | 5 | 7522027CD1 | 25 | 7522027CB1 | 95130578CA2 |
| 7524406 | 6 | 7524406CD1 | 26 | 7524406CB1 | 95078842CA2 |
| 7524922 | 7 | 7524922CD1 | 27 | 7524922CB1 | 95199261CA2 |
| 7524936 | 8 | 7524936CD1 | 28 | 7524936CB1 | 95199421CA2 |
| 7512039 | 9 | 7512039CD1 | 29 | 7512039CB1 | 6997890CA2, 95037210CA2 |
| 7512576 | 10 | 7512576CD1 | 30 | 7512576CB1 | |
| 7514864 | 11 | 7514864CD1 | 31 | 7514864CB1 | 95040511CA2 |
| 8266965 | 12 | 8266965CD1 | 32 | 8266965CB1 | |
| 7515124 | 13 | 7515124CD1 | 33 | 7515124CB1 | 90211068CA2, 90211144CA2, 95033668CA2, 95033744CA2 |
| 7514570 | 14 | 7514570CD1 | 34 | 7514570CB1 | |
| 7515114 | 15 | 7515114CD1 | 35 | 7515114CB1 | 95056530CA2 |
| 7515136 | 16 | 7515136CD1 | 36 | 7515136CB1 | 95056522CA2, 95087603CA2, 95087651CA2 |
| 7515308 | 17 | 7515308CD1 | 37 | 7515308CB1 | 90089513CA2, 90089521CA2, 90089613CA2, 90089629CA2, |
| | | | | | 95133944CA2, 95154891CA2, 95154903CA2 |
| 7516738 | 18 | 7516738CD1 | 38 | 7516738CB1 | 95058033CA2 |
| 7518619 | 19 | 7518619CD1 | 39 | 7518619CB1 | 90147183CA2, 90147251CA2, 95070791CA2 |
| 7513061 | 20 | 7513061CD1 | 40 | 7513061CB1 | |

TABLE 2

| Polypeptide SEQ ID NO: | Incyte Polypeptide ID | GenBank ID NO: or PROTEOME ID NO: | Probability Score | Annotation |
|---|---|---|---|---|
| 1 | 7521032CD1 | g5911792 | 2.7E−53 | [Homo sapiens] mannose-binding lectin |
| | | | | Madsen, H. O. et al. (1998) Different molecular events result in low protein levels of |
| | | | | mannan-binding lectin in populations from southeast Africa and South America. J. |
| | | | | Immunol. 161: 3169-3175. |
| | | 339494\|MBL2 | 2.0E−54 | [Homo sapiens] [Small molecule-binding protein] [Extracellular (excluding cell wall)] |
| | | | | Mannose-binding lectin 2, functions as an opsonin in first line host defense, interacts with |
| | | | | MBP-associated serine protease (MASP); mutation of the corresponding gene impairs |
| | | | | opsonization of mannose-rich pathogens and may cause increased risk of infection |
| | | | | Super, M. et al. (1992) Distinct and overlapping functions of allelic forms of human |
| | | | | mannose binding protein. Nat. Genet. 2: 50-55. |
| | | | | Schuffenecker, I. et al. (1991) The gene for mannose-binding protein maps to chromosome |
| | | | | 10 and is a marker for multiple endocrine neoplasia type 2. Cytogenet. Cell Genet. 56: 99-102. |
| | | | | Summerfield, J. A. et al. (1995) Mannose binding protein gene mutations associated with |
| | | | | unusual and severe infections in adults. Lancet 345: 886-889. |
| | | 585273\|Mbl2 | 7.5E−32 | [Mus musculus] [Small molecule-binding protein] Mannose-binding lectin 2, functions as |
| | | | | an opsonin in first line host defense; mutation of the human MBL2 gene impairs |
| | | | | opsonization of mannose-rich pathogens and may cause increased risk of infection |
| | | | | Sastry, R. et al. (1995) Characterization of murine mannose-binding protein genes Mbl1 and |
| | | | | Mbl2 reveals features common to other collectin genes. Mamm. Genome 6: 103-110. |
| | | | | Sastry, K. et al. (1991) Molecular characterization of the mouse mannose-binding proteins. |
| | | | | The mannose-binding protein A but not C is an acute phase reactant. J. Immunol. 147: 692-697. |
| | | | | Tabona, P. et al. (1995) Mannose binding protein is involved in first-line host defence: |
| | | | | evidence from transgenic mice. Immunology 85: 153-159. |
| 2 | 2936048CD1 | g16797814 | 1.9E−155 | [Drosophila melanogaster] phosphomannomutase 45A |
| | | | | Mohr, S. E. et al. (2001) The RNA-binding protein Tsunagi interacts with Mago Nashi to |
| | | | | establish polarity and localize oskar mRNA during Drosophila oogenesis. Genes Dev. |
| | | | | 15: 2886-2899. |
| | | 598814\|FLJ10983 | 5.6E−201 | [Homo sapiens] [Isomerase] Member of the phosphoglucomutase and |
| | | | | phosphomannomutase family |
| | | 252406\|Y43F4B.5 | 6.9E−132 | [Caenorhabditis elegans] [Isomerase] Protein containing phosphoglucomutase or |
| | | | | phosphomannomutase alpha-beta-alpha domains I and II, has high similarity to |
| | | | | uncharacterized human FLJ10983 |
| 3 | 7521726CD1 | g190464 | 2.2E−108 | [Homo sapiens] protein Z |
| | | | | Ichinose, A. et al. (1990) Amino acid sequence of human protein Z, a vitamin K-dependent |
| | | | | plasma glycoprotein. Biochem. Biophys. Res. Commun. 172: 1139-1144. |
| | | 337238\|PROZ | 1.6E−109 | [Homo sapiens] [Structural protein; Hydrolase; Protease (other than proteasomal); Small |
| | | | | molecule-binding protein] [Extracellular (excluding cell wall)] Protein Z, a vitamin K- |
| | | | | dependent plasma glycoprotein that plays a role in the regulation of blood coagulation, |
| | | | | deficiency may cause a mild bleeding disorder and increased levels may be associated with |
| | | | | ischemic stroke |
| | | | | Ichinose, A. et al. (1990), supra. |
| | | | | Yin, Z. F. et al. (2000) Prothrombotic phenotype of protein Z deficiency. Proc. Natl. Acad. |
| | | | | Sci. USA 97: 6734-6738. |
| | | 582809\|F10 | 1.2E−40 | [Mus musculus] [Hydrolase; Protease (other than proteasomal)] [Extracellular (excluding |
| | | | | cell wall)] Coagulation factor X, a vitamin K-dependent serine protease that converts |
| | | | | prothrombin (F2) into thrombin; high levels of human F10 predict risk of thrombosis, |
| | | | | mutations in the human F10 gene cause the coagulation disorder Factor X deficiency |
| | | | | Liang, Z. et al. (1998) Cloning and characterization of a cDNA encoding murine |
| | | | | coagulation factor X. Thromb. Haemost. 80: 8-91. |
| 4 | 7523383CD1 | g179081 | 1.5E−139 | [Homo sapiens] asialoglycoprotein receptor H2 |
| | | | | Spiess, M. et al. (1985) Sequence of a second human asialoglycoprotein receptor: |
| | | | | conservation of two receptor genes during evolution. Proc. Natl. Acad. Sci. USA 82: 6465-6469. |
| | | 774085\|ASGR2 | 1.1E−140 | [Homo sapiens] [Receptor (signaling)] Asialoglycoprotein receptor 2 (hepatic lectin), binds |
| | | | | and mediates endocytosis of galactose-terminated glycoproteins and may be involved in the |
| | | | | uptake of the hepatitis B virus |
| | | | | Spiess, M. et al. (1985), supra. |
| | | | | Shenkman, M. et al. (2000) Masking of an endoplasmic reticulum retention signal by its |
| | | | | presence in the two subunits of the asialoglycoprotein receptor. J. Biol. Chem. 275: 2845-2851. |
| | | | | Shenkman, M. et al. (1997) Endoplasmic reticulum quality control of asialoglycoprotein |
| | | | | receptor H2a involves a determinant for retention and not retrieval. Proc. Natl. Acad. Sci. |
| | | | | USA 94: 11363-11368. |
| | | | | Tolchinsky, S. et al. Membrane-bound versus secreted forms of human asialoglycoprotein |
| | | | | receptor subunits. Role of a juxtamembrane pentapeptide. J. Biol. Chem. 271: 14496-14503. |
| | | 583627\|Asgr2 | 6.1E−91 | [Mus musculus] [Receptor (signaling)] [Plasma membrane] Asialoglycoprotein receptor 2 |
| | | | | (hepatic lectin), binds and mediates endocytosis of galactose-terminated glycoproteins and |
| | | | | may be involved in the clearance of serum glycoproteins |
| | | | | Takezawa, R. et al. (1993) Determination of mouse major asialoglycoprotein receptor |
| | | | | cDNA sequence. Biochim. Biophys. Acta 1172: 220-222. |
| | | | | Ishibashi, S. et al. (1994) Asialoglycoprotein receptor deficiency in mice lacking the minor |
| | | | | receptor subunit. J. Biol. Chem. 269: 27803-27806. |
| 5 | 7522027CD1 | g190464 | 8.6E−43 | [Homo sapiens] protein Z |
| | | | | Ichinose, A. et al. (1990), supra. |
| | | 337238\|PROZ | 6.2E−44 | [Homo sapiens] [Structural protein; Hydrolase; Protease (other than proteasomal); Small |
| | | | | molecule-binding protein] [Extracellular (excluding cell wall)] Protein Z, a vitamin K- |
| | | | | dependent plasma glycoprotein that plays a role in the regulation of blood coagulation, |
| | | | | deficiency may cause a mild bleeding disorder and increased levels may be associated with |
| | | | | ischemic stroke |
| | | | | Ichinose, A. et al. (1990), supra. |
| | | | | Yin, Z. F. et al. (2000), supra. |
| | | 591039\|F10 | 3.9E−15 | [Rattus norvegicus] [Hydrolase; Protease (other than proteasomal)] [Endoplasmic |
| | | | | reticulum; Cytoplasmic] Coagulation factor X, a vitamin K-dependent serine protease that |
| | | | | converts prothrombin (F2) into thrombin, inhibition may reduce thrombosis; mutations in |
| | | | | human F10 gene cause the coagulation disorder Factor X deficiency |
| 6 | 7524406CD1 | g3153211 | 4.4E−261 | [Homo sapiens] guanosine-diphosphatase like protein |
| | | | | Wang, T. F. et al., Golgi localization and functional expression of human uridine |
| | | | | diphosphatase, J. Biol. Chem. 273, 11392-11399 (1998) |
| | | 340820\|LYSAL1 | 9.6E−261 | [Homo sapiens][Other phosphatase; Hydrolase] [Lysosome/vacuole; Cytoplasmic] |
| | | | | Lysosomal apyrase-like protein (Golgi apyrase), a member of the apyrase or GDA1/CD39 |
| | | | | family that is a lysosomal membrane protein with four apyrase domains, alternative splice |
| | | | | form is identical to uridine diphosphatase |
| | | | | Biederbick, A. et al., First apyrase splice variants have different enzymatic properties, J |
| | | | | Biol Chem 275, 19018-24 (2000). |
| | | 753913\|Lysal2 | 5.9E−158 | [Mus musculus] Protein with high similarity to lysosomal apyrase-like protein (Golgi |
| | | | | apyrase, human LYSAL1), which is a lysosomal membrane protein with four apyrase |
| | | | | domains, member of the GDA1 or CD39 family of nucleoside phosphatases |
| | | | | Shi, J. D. et al., Molecular cloning and characterization of a novel mammalian endo-apyrase |
| | | | | (LALP1)., J Biol Chem 276, 17474-8. (2001). |
| 7 | 7524922CD1 | g4586836 | 2.5E−95 | [Homo sapiens] type II membrane protein similar to HIV gp120-binding C-type lectin |
| | | | | Yokoyama-Kobayashi, M. et al., Selection of cDNAs encoding putative type II membrane |
| | | | | proteins on the cell surface from a human full-length cDNA bank, Gene 228, 161-167 |
| | | | | (1999) |
| | | 800231\|CD209L | 1.3E−89 | [Homo sapiens][Plasma membrane; Unspecified membrane] CD209 antigen-like, a member |
| | | | | of the C-type lectin family, acts as a receptor for ICAM3 and binds to strains of HIV-1, HIV |
| | | | | 2 and simian immunodeficiency virus, promoting their pathogenesis |
| | | | | Pohlmann, S. et al., DC-SIGNR, a DC-SIGN homologue expressed in endothelial cells, |
| | | | | binds to human and simian immunodeficiency viruses and activates infection in trans, Proc |
| | | | | Natl Acad Sci USA 98, 2670-2675. (2001). |
| | | 777160\|Cd209e | 2.6E−22 | [Mus musculus] Protein containing a C-type lectin domain, which mediate calcium- |
| | | | | dependent carbohydrate recognition, has moderate similarity to c-type lectin (calcium |
| | | | | dependent, carbohydrate-recognition domain) superfamily member 9 (mouse Clecsf9) |
| 8 | 7524936CD1 | g15383606 | 7.4E−174 | [Homo sapiens] mDC-SIGN2 type I isoform |
| | | | | Mummidi, S. et al., Extensive repertoire of membrane-bound and soluble dendritic cell- |
| | | | | specific ICAM-3-grabbing nonintegrin 1 (DC-SIGN1) and DC-SIGN2 isoforms. Inter- |
| | | | | individual variation in expression of DC-SIGN transcripts, J. Biol. Chem. 276, 33196-33212 |
| | | | | (2001) |
| | | 800231\|CD209L | 1.2E−173 | [Homo sapiens][Plasma membrane; Unspecified membrane] CD209 antigen-like, a member |
| | | | | of the C-type lectin family, acts as a receptor for ICAM3 and binds to strains of HIV-1, HIV |
| | | | | 2 and simian immunodeficiency virus, promoting their pathogenesis |
| | | | | Feinberg, H. et al., Structural basis for selective recognition of oligosaccharides by DC- |
| | | | | SIGN and DC-SIGNR, Science 294, 2163-6. (2001). |
| | | 777260\|Cd209a | 2.0E−59 | [Mus musculus] Protein containing a C-type lectin domain, which mediate calcium- |
| | | | | dependent carbohydrate recognition, has moderate similarity to c-type lectin (calcium |
| | | | | dependent, carbohydrate-recognition domain) superfamily member 9 (mouse Clecsf9) |
| | | | | Baribaud, F. et al., Functional and antigenic characterization of human, rhesus macaque, |
| | | | | pigtailed macaque, and murine DC-SIGN, J Virol 75, 10281-9. (2001). |
| 9 | 7512039CD1 | g5821288 | 1.1E−36 | [Homo sapiens] macrophage C-type lectin Mincle |
| | | | | Matsumoto, M. et al. A novel LPS-inducible C-type lectin is a transcriptional target of NF- |
| | | | | IL6 in macrophages. J. Immunol. 163, 5039-5048 (1999) |
| | 7512039CD1 | 569282\|CLECSF9 | 8.5E−38 | [Homo sapiens][Small molecule-binding protein] C-type lectin (calcium dependent, |
| | | | | carbohydrate-recognition domain) superfamily member 9, a macrophage-inducible lectin |
| | | | | that may be a downstream target of the transcription factor NF-IL6 (CEBPB) and may be |
| | | | | induced by inflammatory stimuli |
| | | | | Matsumoto, M. et al. A novel LPS-inducible C-type lectin is a transcriptional target of NF- |
| | | | | IL6 in macrophages. J. Immunol. 163, 5039-5048 (1999) |
| | 7512039CD1 | 609080\|Clecsf9 | 2.2E−16 | [Mus musculus][Small molecule-binding protein] C-type lectin (calcium dependent, |
| | | | | carbohydrate-recognition domain) superfamily member 9, a group 2 C-type lectin and |
| | | | | downstream target of the transcription factor NF-IL6 (Cebpb) and may be involved in the |
| | | | | inflammatory response of activated macrophages |
| | | | | Balch, S. G. et al. Organization of the mouse macrophage C-type lectin (Mcl) gene and |
| | | | | identification of a subgroup of related lectin molecules. Eur. J. Immunogenet. 29, 61-64 |
| | | | | (2002) |
| 10 | 7512576CD1 | g11120502 | 3.2E−203 | [Homo sapiens] ERGL (ERGIC-53-like) Yerushalmi, N. et al. ERGL, a novel gene related |
| | | | | to ERGIC-53 that is highly expressed in normal and neoplastic prostate and several other |
| | | | | tissues. Gene 265: 55-60 (2001) |
| | 7512576CD1 | 342592\|LMAN1 | 3.5E−58 | [Homo sapiens][Chaperones; Small molecule-binding protein][Golgi; Endoplasmic |
| | | | | reticulum; Cytoplasmic] Mannose-binding lectin 1, involved in the traffic of glycoproteins |
| | | | | between endoplasmic reticulum and the Golgi apparatus; mutations of the corresponding |
| | | | | gene is associated with combined factor V and VIII coagulation deficiency |
| | | | | Nichols, W. C. et al. Mutations in the ER-Golgi intermediate compartment protein ERGIC- |
| | | | | 53 cause combined deficiency of coagulation factors V and Cell 93, 61-70 (1998) |
| | 7512576CD1 | 757464\|Rn.25734 | 5.7E−58 | [Rattus norvegicus][Chaperones][Golgi; Endoplasmic reticulum; Cytoplasmic; Plasma |
| | | | | membrane] Mannose-binding lectin 1, a mannose-binding lectin that is localized to the |
| | | | | endoplasmic reticulum and Golgi apparatus; mutations in human LMAN1 gene is associated |
| | | | | with combined factor V and VIII coagulation deficiency |
| 11 | 7514864CD1 | g14456615 | 1.2E−238 | [Homo sapiens] phosphatidyl inositol glycan class T |
| | | | | Ohishi, K. et al. PIG-S and PIG-T, essential for GPI-anchor attachment to proteins, form a |
| | | | | complex with GAA1 and GPI8. EMBO J. 20: 4088-4098 (2001) |
| | 7514864CD1 | 476161\|LOC51604 | 5.5E−224 | [Homo sapiens] Protein with low similarity to uncharacterized S. cerevisiae Gpi16p |
| | 7514864CD1 | 786543\|4930534E15Rik | 2.5E−219 | [Mus musculus] Protein having strong similarity to uncharacterized human LOC51604 |
| 12 | 18266965CD1 | g2642187 | 1.7E−111 | [Rattus norvegicus] endo-alpha-D-mannosidase |
| | | | | Spiro, M. J. et al. Molecular cloning and expression of rat liver endo-alpha-mannosidase, |
| | | | | an N-linked oligosaccharide processing enzyme. J. Biol. Chem. 272, 29356-29363 (1997) |
| | 8266965CD1 | 772612\|Enman | 1.3E−112 | [Rattus norvegicus][Hydrolase] Endo-alpha-D-mannosidase, processes N-linked |
| | | | | oligosaccharides, may be involved in processing of a vesicular stomatitis virus envelope |
| | | | | glycoprotein |
| | | | | Spiro, M. J. et al. Molecular cloning and expression of rat liver endo-alpha-mannosidase, |
| | | | | an N-linked oligosaccharide processing enzyme. J. Biol. Chem. 272, 29356-29363 (1997) |
| | 8266965CD1 | 691084\|FLJ12838 | 2.0E−111 | [Homo sapiens] Protein of having strong similarity to a region of endo-alpha-D- |
| | | | | mannosidase (rat Enman), which processes N-linked oligosaccharides |
| 13 | 7515124CD1 | g63426 | 5.7E−30 | [Gallus gallus] lysozyme |
| | | | | Nakano, T. et al. Goose-type lysozyme gene of the chicken: sequence, genomic organization |
| | | | | and expression reveals major differences to chicken-type lysozyme gene. Biochim. Biophys. |
| | | | | Acta 1090: 273-276 (1991). |
| | | 719957\|1gbs_{—} | 6.0E−30 | [Protein Data Bank] Lysozyme (E.C. 3.2.1.17) |
| | | 717585\|153l_{—} | 9.8E−30 | [Protein Data Bank] Lysozyme (E.C. 3.2.1.17) |
| 14 | 7514570CD1 | g6651065 | 4.8E−84 | [Homo sapiens] lectin-like NK cell receptor LLT1 |
| | | | | Boles, K. S. et al. Cloning of a new lectin-like receptor expressed on human NK cells. |
| | | | | Immunogenetics 50: 1-7 (1999). |
| | | 432906\|LLT1 | 3.8E−85 | [Homo sapiens] [Receptor (signaling)] [Unspecified membrane; Plasma membrane] Lectin |
| | | | | like transcript-1 (lectin-like NK cell receptor), contains carbohydrate recognition domain of |
| | | | | C type lectins but not immunoreceptor tyrosine based inhibitory motifs, may be involved in |
| | | | | mediating lymphocyte activation signals |
| | | | | Boles, K. S. et al. (1999), supra. |
| | | | | Eichler, W. et al. Differentially induced expression of C-type lectins in activated |
| | | | | lymphocytes. J. Cell. Biochem. 81: 201-208 (2001). |
| | | 749778\|Dcl1 | 6.0E−30 | [Mus musculus] [Small molecule-binding protein] C-type lectin 1, protein containing a C- |
| | | | | type lectin (CTL, CRD) domain, which may mediate calcium-dependent carbohydrate |
| | | | | binding |
| | | | | Marzio, R. et al. Expression and function of the early activation antigen CD69 in murine |
| | | | | macrophages. J. Leukoc. Biol. 62: 349-355 (1997). |
| 15 | 7515114CD1 | g11120502 | 8.2E−229 | [Homo sapiens] ERGL |
| | | | | Yerushalmi, N. et al. ERGL, a novel gene related to ERGIC-53 that is highly expressed in |
| | | | | normal and neoplastic prostate and several other tissues. Gene 265: 55-60 (2001). |
| | | 623710\|ERGL | 6.6E−230 | [Homo sapiens] Protein with low similarity to mannose-binding lectin 1 (human LMAN1), |
| | | | | which is involved in the traffic of glycoproteins between endoplasmic reticulum and the |
| | | | | Golgi apparatus and is associated with combined factor V and VIII coagulation deficiency |
| | | 757464\|Lman1 | 9.7E−55 | [Rattus norvegicus] [Chaperones] [Golgi; Endoplasmic reticulum; Cytoplasmic; Plasma |
| | | | | membrane] Mannose-binding lectin 1, a mannose-binding lectin that is localized to the |
| | | | | endoplasmic reticulum and Golgi apparatus; mutations in human LMAN1 gene is associated |
| | | | | with combined factor V and VIII coagulation deficiency |
| | | | | Lahtinen, U. et al. Molecular cloning and expression of a 58-kDa cis-Golgi and intermediate |
| | | | | compartment protein. J. Biol. Chem. 271: 4031-4037 (1996). |
| | | | | Lahtinen, U. et al. Mapping of structural determinants for the oligomerization of p58, a |
| | | | | lectin-like protein of the intermediate compartment and cis-Golgi. Eur. J. Biochem. 260: 392-397 |
| | | | | (1999). |
| 16 | 7515136CD1 | g11120502 | 3.0E−242 | [Homo sapiens] ERGL |
| | | | | Yerushalmi, N. et al. (2001), supra. |
| | | 623710\|ERGL | 2.4E−243 | [Homo sapiens] Protein with low similarity to mannose-binding lectin 1 (human LMAN1), |
| | | | | which is involved in the traffic of glycoproteins between endoplasmic reticulum and the |
| | | | | Golgi apparatus and is associated with combined factor V and VIII coagulation deficiency |
| | | 757464\|Lman1 | 2.7E−66 | [Rattus norvegicus] [Chaperones] [Golgi; Endoplasmic reticulum; Cytoplasmic; Plasma |
| | | | | membrane] Mannose-binding lectin 1, a mannose-binding lectin that is localized to the |
| | | | | endoplasmic reticulum and Golgi apparatus; mutations in human LMAN1 gene is associated |
| | | | | with combined factor V and VIII coagulation deficiency |
| | | | | Lahtinen, U. et al. (1996), supra. |
| | | | | Lahtinen, U. et al. (1999), supra. |
| 17 | 7515308CD1 | g6502535 | 3.0E−107 | [Homo sapiens] C-type lectin superfamily 6 |
| | | | | Richard, M. et al. The expression pattern of the ITIM-bearing lectin CLECSF6 in |
| | | | | neutrophils suggests a key role in the control of inflammation. J. Leukoc. Biol. 71: 871-880 |
| | | | | (2002). |
| | | 475349\|CLECSF6 | 3.0E−108 | [Homo sapiens] [Receptor (signaling)] [Plasma membrane] C-type (calcium dependent |
| | | | | carbohydrate-recognition domain) lectin superfamily member 6, contains immunoreceptor |
| | | | | tyrosine-based inhibitory motif (ITIM), upregulated upon dendritic cell differentiation, and |
| | | | | downregulated upon dendritic cell maturation |
| | | | | Bates, E. E. et al. APCs express DCIR, a novel C-type lectin surface receptor containing an |
| | | | | immunoreceptor tyrosine-based inhibitory motif. J. Immunol. 163: 1973-1983 (1999). |
| | | 429718\|Clecsf6 | 3.0E−51 | [Mus musculus] [Receptor (signaling)] [Plasma membrane] C-type lectin (calcium |
| | | | | dependent, carbohydrate recognition domain) superfamily member 6, a type II membrane |
| | | | | glycoprotein, contains a calcium-binding domain, a carbohydrate recognition domain and an |
| | | | | immunoreceptor tyrosine-based inhibitory motif |
| | | | | Bates, E. E. et al. (1999), supra. |
| | | | | Balch, S. G. et al. Organization of the mouse macrophage C-type lectin (Mcl) gene and |
| | | | | identification of a subgroup of related lectin molecules. Eur. J. Immunogenet. 29: 61-64 |
| | | | | (2002). |
| 18 | 7516738CD1 | g34767 | 2.1E−138 | [Homo sapiens] lung surfactant protein D |
| | | | | Lu, J. et al. Purification, characterization and cDNA cloning of human lung surfactant |
| | | | | protein D. Biochem. J. 284: 795-802 (1992). |
| | | 344816\|SFTPD | 3.0E−138 | [Homo sapiens] [Small molecule-binding protein] Surfactant (pulmonary-associated) |
| | | | | protein D, member of the C-type lectin family with collagen-like structure; may play a role |
| | | | | in defense against inhaled microorganisms including Klebsiella pneumonia, Mycobacterium |
| | | | | tuberculosis and influenza type A viruses |
| | | | | Crouch, E. et al. Genomic organization of human surfactant protein D (SP-D). SP-D is |
| | | | | encoded on chromosome 10q22.2-23.1. J. Biol. Chem. 268: 2976-2983 (1993). |
| | | | | Lu, J. et al. (1992), supra. |
| | | | | Rust, K. et al. Human surfactant protein D: SP-D contains a C-type lectin carbohydrate |
| | | | | recognition domain. Arch. Biochem. Biophys. 290: 116-126 (1991). |
| | | 581327\|Sftpd | 1.3E−112 | [Mus musculus] Surfactant protein D, a glycoprotein which neutralizes infection of |
| | | | | pulmonary system by influenza virus; binds glucose |
| | | | | Motwani, M. et al. Mouse surfactant protein-D. cDNA cloning, characterization, and gene |
| | | | | localization to chromosome 14. J. Immunol. 155: 5671-5677 (1995). |
| | | | | Botas, C. et al. Altered surfactant homeostasis and alveolar type II cell morphology in mice |
| | | | | lacking surfactant protein D. Proc. Natl. Acad. Sci. USA 95: 11869-11874(1998). |
| | | | | Wert, S. E. et al. Increased metalloproteinase activity, oxidant production, and emphysema |
| | | | | in surfactant protein D gene-inactivated mice. Proc. Natl. Acad. Sci. USA 97: 5972-5977 |
| | | | | (2000). |
| | | | | LeVine, A. M. et al. Distinct effects of surfactant protein A or D deficiency during bacterial |
| | | | | infection on the lung. J. Immunol. 165: 3934-3940(2000). |
| | | | | Bridges, J. P. et al. Pulmonary surfactant proteins A and D are potent endogenous inhibitors |
| | | | | of lipid peroxidation and oxidative cellular injury. J. Biol. Chem. 275: 38848-38855 (2000). |
| 19 | 7518619CD1 | g5577966 | 3.1E−73 | [Homo sapiens] Po66 carbohydrate binding protein |
| | | 567904\|LGALS8 | 8.1E−74 | [Homo sapiens][Small molecule-binding protein][Cytoplasmic; Extracellular (excluding cell |
| | | | | wall)] Galectin 8 (Po66 Carbohydrate Binding Protein), a member of the galectin |
| | | | | galactoside-binding lectin family, a prostate tumor antigen that is involved in cell adhesion |
| | | | | and cell growth regulation, expressed in prostate and lung tumors |
| | | | | Levy, Y. et al., Galectin-8 functions as a matricellular modulator of cell adhesion., J Biol |
| | | | | Chem 276, 31285-95. (2001). |
| | 7518619CD1 | 757434\|Lgals8 | 1.4E\|62 | [Rattus norvegicus][Small molecule-binding protein][Cytoplasmic] Galectin 8, a member of |
| | | | | a family of lectins that bind to beta galactoside, a secreted form may be matrix-associated, |
| | | | | interacts with integrins to modulate cell adhesion, migration, and apoptosis |
| | | | | Hadari, Y. R. et al., Galectin-8 binding to integrins inhibits cell adhesion and induces |
| | | | | apoptosis., J Cell Sci, 2385-97. (2000). |
| 20 | 7513061CD1 | g474308 | 5.2E−55 | [Homo sapiens] regenerating protein I beta |
| | | | | Moriizumi, S. et al., Isolation, structural determination and expression of a novel reg gene, |
| | | | | human regI beta, Biochim. Biophys. Acta 1217, 199-202 (1994) |
| | | 617870\|REG1B | 4.0E−56 | [Homo sapiens] Regenerating islet-derived 1 beta, a putative growth factor that may play a |
| | | | | role in the regeneration of pancreatic islet cells, expressed only in the pancreas; |
| | | | | corresponding gene is overexpressed during colorectal carcinogenesis |
| | | | | Rechreche, H. et al., pap, reg Ialpha and reg Ibeta mRNAs are concomitantly up-regulated |
| | | | | during human colorectal carcinogenesis., Int J Cancer 81, 688-94. (1999). |
| | | 337572\|REG1A | 2.6E−52 | [Homo sapiens] Regenerating islet-derived 1 alpha (pancreatic stone protein, pancreatic |
| | | | | thread protein), induces pancreatic beta cell regeneration and ameliorates diabetes in animal |
| | | | | models; reduced expression is observed in chronic calcifying pancreatitis |
| | | | | Zenilman, M. E. et al., Effect of reg protein on rat pancreatic ductal cells., Pancreas 17, 256-61 |
| | | | | (1998). |

TABLE 3

| SEQ | Incyte | Amino | | |
|---|---|---|---|---|
| ID | Polypeptide | Acid | | Analytical Methods |
| NO: | ID | Residues | Signature Sequences, Domains and Motifs | and Databases |
| 1 | 7521032CD1 | 108 | signal_cleavage: M1-S20 | SPSCAN |
| | | | Signal Peptide: M1-S18, M1-S20, M1-T22, M1-T24 | HMMER |
| | | | Collagen triple helix repeat (20 copies): G42-S100 | HMMER_PFAM |
| | | | Complement Clq protein | BLIMPS_BLOCKS |
| | | | IPB001073: G74-G98 | |
| | | | Bindin precursor signature | BLIMPS_PRINTS |
| | | | PR00761: G60-L76 | |
| | | | SIMILAR TO CUTICULAR COLLAGEN | BLAST_PRODOM |
| | | | PD067228: D27-P105 | |
| | | | COLLAGEN ALPHA PRECURSOR CHAIN REPEAT SIGNAL CONNECTIVE TISSUE | BLAST_PRODOM |
| | | | EXTRACELLULAR MATRIX | |
| | | | PD000007: P41-K103 | |
| | | | PRECOLLAGEN P PRECURSOR SIGNAL | BLAST_PRODOM |
| | | | PD072959: N44-K103 | |
| | | | MANNOSE-BINDING PROTEIN C CHAIN | BLAST_DOMO |
| | | | DM07371\|P11226\|1-117: M1-S106 | |
| | | | DM07371\|P41317\|1-113: M1-D96 | |
| | | | FIBRILLAR COLLAGEN CARBOXYL-TERMINAL | BLAST_DOMO |
| | | | DM00019\|P53420\|1298-1453: P41-P97 | |
| | | | MANNOSE-BINDING LECTIN | BLAST_DOMO |
| | | | DM01663\|P12842\|1-116: L10-G98 | |
| | | | Potential Phosphorylation Sites: | MOTIFS |
| | | | S18 T24 T55 | |
| 2 | 2936048CD1 | 622 | signal_cleavage: M53-G77 | SPSCAN |
| | | | Phosphoglucomutase/phosphomannomutase, alpha/beta/alpha domain I: | HMMER_PFAM |
| | | | L61-W217 | |
| | | | Phosphoglucomutase and phosphomannomutase family | BLIMPS_BLOCKS |
| | | | IPB001485: F67-A78, R109-R118, G169-G183, R326-R337, N561-M582 | |
| | | | Phosphoglucomutase/phosphomannomutase family signature | BLIMPS_PRINTS |
| | | | PR00509: A168-N182, T257-A276, A325-A340 | |
| | | | ISOMERASE PHOSPHORYLATION PHOSPHOMANNOMUTASE PROTEIN | BLAST_PRODOM |
| | | | PHOSPHOGLUCOMUTASE PMM GLUCOSE PHOSPHOMUTASE PGM BIOSYNTHESIS | |
| | | | PD000667: R64-K488, F559-E581 | |
| | | | PHOSPHOGLUCOMUTASE AND PHOSPHOMANNOMUTASE PHOSPHOSERINE | BLAST_DOMO |
| | | | DM00656\|S54585\|52-393: R64-A398 | |
| | | | DM00656\|I64157\|3-299: I83-F403 | |
| | | | DM00656\|P47723\|38-360: M65-E406 | |
| | | | DM00656\|P47299\|40-362: F67-G402 | |
| | | | Potential Phosphorylation Sites: | MOTIFS |
| | | | S127 S196 S216 S311 S316 S390 S444 S462 S502 S512 S570 T5 T257 T300 T331 T353 T470 | |
| | | | T499 T533 T566 Y184 Y484 | |
| | | | Potential Glycosylation Sites: | MOTIFS |
| | | | N214 N372 N468 | |
| 3 | 7521726CD1 | 210 | signal_cleavage: M1-P22 | SPSCAN |
| | | | Signal Peptide: M1-P22, M1-F25 | HMMER |
| | | | Epidermal growth factor-like domain: P90-E123, E128-V166 | HMMER_SMART |
| | | | Calcium-binding EGF-like domain: C91-E123, A125-V166 | HMMER_SMART |
| | | | Domain containing Gla (gamma-carboxyglutamate) residues: P22-K86 | HMMER_SMART |
| | | | EGF-like domain: C91-C122, C129-C165 | HMMER_PFAM |
| | | | Vitamin K-dependent carboxylation/gamma-carboxyglutamic (GLA) domain: | HMMER_PFAM |
| | | | L46-G87 | |
| | | | EGF-like domain | BLIMPS_BLOCKS |
| | | | IPB000561: C111-G119 | |
| | | | Calcium-binding EGF-like domain | BLIMPS_BLOCKS |
| | | | IPB001881: C102-C113 | |
| | | | Vitamin K-dependent carboxylation domain: V24-Q103 | PROFILESCAN |
| | | | Coagulation factor GLA domain signature | BLIMPS_PRINTS |
| | | | PR00001: L45-C58, Y59-F72, E73-G87 | |
| | | | Type II EGF-like signature | BLIMPS_PRINTS |
| | | | PR00010: G87-H98, N99-I106, W107-Y117, E118-L124 | |
| | | | GLA DOMAIN | BLAST_DOMO |
| | | | DM00454\|P22891\|2-81: A2-W82 | |
| | | | EGF | BLAST_DOMO |
| | | | DM00003\|P22891\|127-171: N127-A172 | |
| | | | DM00003\|P22891\|83-125: R83-K126 | |
| | | | DM00003\|P00744\|87-132: N127-C173 | |
| | | | Potential Phosphorylation Sites: | MOTIFS |
| | | | S101 S120 S178 | |
| | | | Potential Glycosylation Sites: | MOTIFS |
| | | | N99 | |
| | | | Aspartic acid and asparagine hydroxylation site: | MOTIFS |
| | | | C102-C113 | |
| | | | EGF-like domain signature 1: | MOTIFS |
| | | | C111-C122 | |
| | | | EGF-like domain signature 2: | MOTIFS |
| | | | C111-C122, C150-C165 | |
| | | | Vitamin K-dependent carboxylation domain: | MOTIFS |
| | | | L45-W82 | |
| 4 | 7523383CD1 | 248 | C-type lectin (CTL) or carbohydrate-recognition domain (CRD): | HMMER_SMART |
| | | | C114-G238 | |
| | | | Lectin C-type domain: S131-K239 | HMMER_PFAM |
| | | | C-type lectin domain | BLIMPS_BLOCKS |
| | | | IPB001304: W118-C142, W171-W183 | |
| | | | C-type lectin domain signature and profile: N192-A248 | PROFILESCAN |
| | | | LECTIN RECEPTOR GLYCOPROTEIN TRANSMEMBRANE ASIALOGLYCOPROTEIN | BLAST_PRODOM |
| | | | CALCIUM SIGNAL ANCHOR HEPATIC ENDOCYTOSIS PHOSPHORYLATION | |
| | | | PD005101: K3-Q25, Q22-K140, A24-D81 | |
| | | | C-TYPE LECTIN | BLAST_DOMO |
| | | | DM00035\|P07307\|170-301: N107-K239 | |
| | | | DM00035\|P24721\|163-294: S106-K239 | |
| | | | DM00035\|P07306\|146-276: N107-C237 | |
| | | | DM00035\|P34927\|145-276: N107-C237 | |
| | | | Potential Phosphorylation Sites: | MOTIFS |
| | | | S12 S32 S57 S109 S131 S154 S180 T44 T175 T199 Y141 Y189 | |
| | | | Potential Glycosylation Sites: | MOTIFS |
| | | | N39 N107 N242 | |
| | | | C-type lectin domain signature: | MOTIFS |
| | | | C215-C237 | |
| 5 | 7522027CD1 | 97 | signal_cleavage: M1-P22 | SPSCAN |
| | | | Signal Peptide: M1-P22, M1-F25 | HMMER |
| | | | Domain containing Gla (gamma-carboxyglutamate) residues: P22-K86 | HMMER_SMART |
| | | | Vitamin K-dependent carboxylation/gamma-carboxyglutamic (GLA) domain: | HMMER_PFAM |
| | | | L46-G87 | |
| | | | Vitamin K-dependent carboxylation domain: V24-K88 | PROFILESCAN |
| | | | Coagulation factor GLA domain signature | BLIMPS_PRINTS |
| | | | PR00001: L45-C58, Y59-F72, E73-G87 | |
| | | | GLA DOMAIN | BLAST_DOMO |
| | | | DM00454\|P22891\|2-81: A2-W82 | |
| | | | DM00454\|P00744\|1-41: A41-W82 | |
| | | | DM00454\|P00743\|2-80: A2-W82 | |
| | | | DM00454\|S49075\|2-80: P6-W82 | |
| | | | Vitamin K-dependent carboxylation domain: | MOTIFS |
| | | | L45-W82 | |
| 6 | 7524406CD1 | 479 | GDA1/CD39 (nucleoside phosphatase) family: T80-K479 | HMMER_PFAM |
| | | | Cytosolic domain: M1-I34 | TMHMMER |
| | | | Transmembrane domain: M35-I54 | |
| | | | Non-cytosolic domain: R55-K479 | |
| | | | GDA1/CD39 family of nucleoside phosphatase IPB000407: I91-Y105, P173-R183, I217-E238, | BLIMPS_BLOCKS |
| | | | G268-Y281 | |
| | | | HYDROLASE TRANSMEMBRANE PROTEIN NUCLEOSIDE CD39 NUCLEOSIDE | BLAST_PRODOM |
| | | | TRIPHOSPHATASE TRIPHOSPHATE NTPASE PRECURSOR ATP | |
| | | | DIPHOSPHOHYDROLASE PD003822: N86-K479 | |
| | | | LYSOSOMAL APYRASE-LIKE PLASMID KIAA0392 HYDROLASE | BLAST_PRODOM |
| | | | 1200014F22RIK LALP70 GUANOSINE-DIPHOSPHATASE LALP1 | |
| | | | PD070805: M1-P85 | |
| | | | ACTIVATION; NUCLEOSIDE; ANTIGEN; LYMPHOID | BLAST_DOMO |
| | | | DM02628\|P40009\|1-462: N84-S471 | |
| | | | DM02628\|I56242\|40-471: N86-F464 | |
| | | | DM02628\|P49961\|40-471: N86-F464 | |
| | | | DM02628\|P32621\|84-517: Y89-G235 T266-Y427 | |
| | | | Potential Phosphorylation Sites: S212, S218, S471, T75, T144, T266, Y175, Y469 | MOTIFS |
| | | | Potential Glycosylation Sites: N86, N396, N399 | MOTIFS |
| 7 | 7524922CD1 | 222 | signal_cleavage: M1-S24 | SPSCAN |
| | | | C-type lectin domain IPB001304: W170-C194 | BLIMPS_BLOCKS |
| | | | C-TYPE LECTIN | BLAST_DOMO |
| | | | DM00035\|A46274\|248-377: A158-Q210 | |
| | | | GP120; C-TYPE; LECTIN; HIV | BLAST_DOMO |
| | | | DM01861\|A46274\|205-246: E92-A134 | |
| | | | DM01861\|A46274\|205-246: E69-A111 | |
| | | | DM01861\|A46274\|205-246: E46-A88 E115-K156 | |
| | | | DM01861\|A46274\|205-246: E26-A65 | |
| | | | Potential Phosphorylation Sites: S28, S48, S183, T157, T206 | MOTIFS |
| | | | Potential Glycosylation Sites: N36 | MOTIFS |
| 8 | 7524936CD1 | 370 | Lectin C-type domain: S256-K362 | HMMER_PFAM |
| | | | C-type lectin (CTL) or carbohydrate-recognition domain: C239-K361 | HMMER_SMART |
| | | | C-type lectin domain IPB001304: W243-C267, W298-W310 | BLIMPS_BLOCKS |
| | | | C-TYPE LECTIN | BLAST_DOMO |
| | | | DM00035\|A46274\|248-377: A231-K361 | |
| | | | DM00035\|P02707\|74-202: C236-K362 | |
| | | | DM00035\|P20693\|179-304: C236-C360 | |
| | | | DM00035\|P34927\|145-276: E233-K361 | |
| | | | Potential Phosphorylation Sites: S55, S75, S256, S293, S334, T230, T279, T290 | MOTIFS |
| | | | Potential Glycosylation Sites: N63, N332 | MOTIFS |
| | | | C-type lectin domain signature: C339-C360 | MOTIFS |
| 9 | 7512039CD1 | 77 | signal_cleavage: M1-C42 | SPSCAN |
| | | | Signal Peptide: M21-C42, M21-V44, M21-T45 | HMMER |
| | | | Cytosolic domain: T40-M77 | TMHMMER |
| | | | Transmembrane domain: Q20-I39 | |
| | | | Non-cytosolic domain: M1-S19 | |
| | | | Potential Phosphorylation Sites: S3 T12 T45 T51 | MOTIFS |
| | | | Potential Glycosylation Sites: N2 N62 | MOTIFS |
| 10 | 7512576CD1 | 415 | signal_cleavage: M1-P23 | SPSCAN |
| | | | Signal Peptide: M1-P23, M1-T25, M1-C27, M1-G26 | HMMER |
| | | | Legume-like lectin family: R31-E226 | HMMER_PFAM |
| | | | PROTEIN PRECURSOR SIGNAL LECTIN TRANSMEMBRANE ER-GOLGI | BLAST_PRODOM |
| | | | INTERMEDIATE COMPARTMENT TRANSPORT GOLGI PD010000: | |
| | | | P9-V216 S204-P232 | |
| | | | LUMENAL DOMAIN DM06797 | BLAST_DOMO |
| | | | \|P49257\|1-341: P9-D215 S204-K300 | |
| | | | \|P49256\|1-355: G6-L198 D212-E230 | |
| | | | Potential Phosphorylation Sites: S38 S72 S89 S140 S209 | MOTIFS |
| | | | S223 S267 S351 S372 S379 T207 T259 T264 T397 | |
| | | | Potential Glycosylation Sites: N75 N377 | MOTIFS |
| 11 | 7514864CD1 | 441 | signal_cleavage: M1-A23 | SPSCAN |
| | | | Signal Peptide: M5-C21, M5-A23, M1-C21, M1-A23, M1-P25 | HMMER |
| | | | Cytosolic domain: N408-L441 | TMHMMER |
| | | | Transmembrane domain: F385-Y407 | |
| | | | Non-cytosolic domain: M1-D384 | |
| | | | PROTEIN F17C11.7 IKI1ERG9 INTERGENIC REGION TRANSMEMBRANE | BLAST_PRODOM |
| | | | PD043344: H278-V329 L371-G404 | |
| | | | Potential Phosphorylation Sites: S29 T52 T130 T166 T174 T266 Y150 | MOTIFS |
| | | | Potential Glycosylation Sites: N154 N190 | MOTIFS |
| 12 | 8266965CD1 | 283 | Signal Peptide: M1-A22 | HMMER |
| | | | ENDO-ALPHA-D-MANNOSIDASE PD141586: M1-K278 | BLAST_PRODOM |
| | | | Potential Phosphorylation Sites: S117 S192 S229 T61 T101 T121 T219 T237 T267 | MOTIFS |
| 13 | 7515124CD1 | 159 | signal_cleavage: M1-A18 | SPSCAN |
| | | | Signal Peptide: M1-A18, M1-E20 | HMMER |
| | | | Lysozyme G signature | BLIMPS_PRINTS |
| | | | PR00749: L13-Q34, M37-L55, D138-G158 | |
| | | | LYSOZYME G 4-BETA-N-ACETYLMURAMIDASE GOOSE-TYPE HYDROLASE | BLAST_PRODOM |
| | | | GLYCOSIDASE BACTERIOLYTIC ENZYME EGG WHITE | |
| | | | PD016787: L11-F159 | |
| | | | LYSOZYME G | BLAST_DOMO |
| | | | DM07376\|P00718\|1-184: L11-F159 | |
| | | | DM07376\|P27042\|27-210: L11-F159 | |
| | | | Potential Phosphorylation Sites: | MOTIFS |
| | | | S19, S56, S59, S135, T100 | |
| 14 | 7514570CD1 | 154 | C-type lectin (CTL) or carbohydrate-recognition domain(CRD): C38-S148 | HMMER_SMART |
| | | | Lectin C-type domain: D55-K149 | HMMER_PFAM |
| | | | C-type lectin domain | BLIMPS_BLOCKS |
| | | | IPB001304: W42-C66, W95-K107 | |
| | | | C-TYPE LECTIN LECTIN | BLAST_PRODOM |
| | | | PD170863: S68-H153 | |
| | | | HEPATIC LECTIN HOMOLOG BAMHI ORF8 EARLY PROTEIN | BLAST_PRODOM |
| | | | PD058228: C38-R100 | |
| | | | C-TYPE LECTIN | BLAST_DOMO |
| | | | DM00035\|Q07108\|78-195: A36-K149 | |
| | | | DM00035\|P37217\|78-195: A36-K149 | |
| | | | DM00035\|P34927\|145-276: Q35-F118 | |
| | | | DM00035\|P07306\|146-276: Q35-C147 | |
| | | | Potential Phosphorylation Sites: | MOTIFS |
| | | | S62, S78, S136, S148, T12, T141 | |
| | | | Potential Glycosylation Sites: | MOTIFS |
| | | | N58, N110 | |
| 15 | 7515114CD1 | 431 | Signal Peptide: M1-P23, M1-T25, M1-G26, M1-C27 | HMMER |
| | | | Legume-like lectin family: R31-G245 | HMMER_PFAM |
| | | | PROTEIN PRECURSOR SIGNAL LECTIN TRANSMEMBRANE ER GOLGI | BLAST_PRODOM |
| | | | INTERMEDIATE COMPARTMENT TRANSPORT GOLGI | |
| | | | PD010000: P9-E241 | |
| | | | LUMENAL DOMAIN | BLAST_DOMO |
| | | | DM06797\|P49257\|1-341: P9-K316 | |
| | | | DM06797\|P49256\|1-355: G6-A239 | |
| | | | Potential Phosphorylation Sites: | MOTIFS |
| | | | S38, S72, S89, S140, S283, S367, S388, S395, T207, T275, T280, T335, T413 | |
| | | | Potential Glycosylation Sites: | MOTIFS |
| | | | N75, N393 | |
| 16 | 7515136CD1 | 442 | Signal Peptide: M1-P23, M1-T25, M1-G26, M1-C27 | HMMER |
| | | | Legume-like lectin family: R31-E254 | HMMER_PFAM |
| | | | PROTEIN PRECURSOR SIGNAL LECTIN | BLAST_PRODOM |
| | | | TRANSMEMBRANE ER GOLGI INTERMEDIATE COMPARTMENT | |
| | | | TRANSPORT GOLGI | |
| | | | PD010000: P9-P260 | |
| | | | LUMENAL DOMAIN | BLAST_DOMO |
| | | | DM06797\|P49257\|1-341: P9-K328 | |
| | | | DM06797\|P49256\|1-355: G6-E258 | |
| | | | Potential Phosphorylation Sites: | MOTIFS |
| | | | S38, S72, S89, S140, S251, S295, S379, S406, T207, T237, T287, T292, T424 | |
| | | | Potential Glycosylation Sites: | MOTIFS |
| | | | N75, N404 | |
| 17 | 7515308CD1 | 198 | C-type lectin (CTL) or carbohydrate-recognition domain (CRD): C67-E192 | HMMER_SMART |
| | | | Lectin C-type domain: E84-M193 | HMMER_PFAM |
| | | | C-type lectin domain | BLIMPS_BLOCKS |
| | | | IPB001304: W71-C95, F125-W137 | |
| | | | Pancreatitis-associated protein signature | BLIMPS_PRINTS |
| | | | PR01504: E61-Y79, W88-Q111, W137-R155, C164-W178, D180-M194 | |
| | | | C-TYPE LECTIN | BLAST_DOMO |
| | | | DM00035\|P24721\|163-294: C66-E192 | |
| | | | DM00035\|P07307\|170-301: S65-E192 | |
| | | | DM00035\|P34927\|145-276: S65-E192 | |
| | | | DM00035\|P07306\|146-276: S65-E192 | |
| | | | Potential Phosphorylation Sites: | MOTIFS |
| | | | S87, S91, S129, S172, S189, T6, T41, T42, T48, T107 | |
| | | | Potential Glycosylation Sites: | MOTIFS |
| | | | N146 | |
| | | | C-type lectin domain signature: | MOTIFS |
| | | | C164-C191 | |
| 18 | 7516738CD1 | 336 | signal_cleavage: M1-T13 | SPSCAN |
| | | | Signal Peptide: M1-P15, M1-L19, M1-A21, M23-G46 | HMMER |
| | | | C-type lectin (CTL) or carbohydrate-recognition domain (CRD): F215-E335 | HMMER_SMART |
| | | | Collagen triple helix repeat (20 copies): G49-V108, G118-K177 | HMMER_PFAM |
| | | | Lectin C-type domain: F231-F336 | HMMER_PFAM |
| | | | C-type lectin domain signature and profile: E291-F336 | PROFILESCAN |
| | | | LECTIN HYDROXYLATION GLYCOPROTEIN COLLAGEN REPEAT CALCIUM | BLAST_PRODOM |
| | | | PRECURSOR SIGNAL PULMONARY SURFACTANT-ASSOCIATED | |
| | | | PD010466: D184-L241 | |
| | | | PRECOLLAGEN P PRECURSOR SIGNAL | BLAST_PRODOM |
| | | | PD072959: G61-P156, G64-D167, E44-P132, A81-G175, D101-P183 | |
| | | | COLLAGEN ALPHA PRECURSOR CHAIN REPEAT SIGNAL CONNECTIVE TISSUE | BLAST_PRODOM |
| | | | EXTRACELLULAR MATRIX | |
| | | | PD000007: E44-G118, G46-E134, G55-G142, P66-G151, P78-D173, G91-G181, G103-P183 | |
| | | | SIMILAR TO CUTICULAR COLLAGEN | BLAST_PRODOM |
| | | | PD067228: G46-G130, R50-G145, P86-G175, G103-P183 | |
| | | | C-TYPE LECTIN | BLAST_DOMO |
| | | | DM00035\|P35247\|236-374: G197-F336 | |
| | | | DM00035\|P50404\|220-373: L182-F336 | |
| | | | DM00035\|P35248\|220-373: L182-F336 | |
| | | | FIBRILLAR COLLAGEN CARBOXYL-TERMINAL | BLAST_DOMO |
| | | | DM00019\|P35247\|56-234: G46-Q196, R56-G181, G70-L182, G103-G197 | |
| | | | Potential Phosphorylation Sites: S27, S41, S92, S187, S220, S251, S254, S275, S279 | MOTIFS |
| | | | Potential Glycosylation Sites: N90 | MOTIFS |
| | | | C-type lectin domain signature: C312-C334 | MOTIFS |
| 19 | 7518619CD1 | 258 | Signal_cleavage: M1-A43 | SPSCAN |
| | | | Galectin: L126-W258, I17-S125 | HMMER_SMART |
| | | | Galactoside-binding lectin: N133-S257, P18-L132 | HMMER_PFAM |
| | | | Galactoside-binding lectin, galectin IPB001079: I76-I105 | BLIMPS_BLOCKS |
| | | | LECTIN GALAPTIN REPEAT PROTEIN BETAGALACTOSIDE-BINDING BINDING | BLAST_PRODOM |
| | | | ACETYLATION LACTOSE-BINDING GALECTIN MULTIGENE PD000676: | |
| | | | G37-E147, G146- | |
| | | | GALECTIN8 GALAPTIN LECTIN REPEAT PROSTATE CARCINOMA TUMOR ANTIGEN | BLAST_PRODOM |
| | | | PCTA1 STYPE PD030140: M2-I35 | |
| | | | GALECTIN8 GALAPTIN LECTIN REPEAT PROSTATE CARCINOMA TUMOR ANTIGEN | BLAST_PRODOM |
| | | | PCTA1 STYPE PD020379: V116-E147 | |
| | | | VERTEBRATE GALACTOSIDE-BINDING LECTIN DM00426\|A55975\|13-150: | BLAST_DOMO |
| | | | L124-W258 N14-F155 S125-L252 | |
| | | | VERTEBRATE GALACTOSIDE-BINDING LECTIN DM00426\|A55664\|188-323: | BLAST_DOMO |
| | | | L126-R256 Y13-V148 | |
| | | | VERTEBRATE GALACTOSIDE-BINDING LECTIN DM00426\|P479671\|11-143: | BLAST_DOMO |
| | | | L124-S257 N14-V148 | |
| | | | Potential Phosphorylation Sites: S41 S55 S189 S239 T22 Y93 | MOTIFS |
| | | | Potential Glycosylation Sites: N52 N196 | MOTIFS |
| | | | Lectin_Galactoside: W190-M210 | MOTIFS |
| 20 | 7513061CD1 | 132 | Signal_cleavage: M1-G22 | SPSCAN |
| | | | Signal Peptide: M1-G22 | HMMER |
| | | | Signal Peptide: M1-S25 | HMMER |
| | | | Signal Peptide: M1-E24 | HMMER |
| | | | C-type lectin (CTL) or carbohydrate-recognition domain: C36-K128 | HMMER_SMART |
| | | | C-type lectin domain IPB001304: T40-C64, W99-N111 | BLIMPS_BLOCKS |
| | | | Peroxidases signatures: V73-S126 | PROFILESCAN |
| | | | Pancreatitis-associated protein signature PR01504: P30-Y48, W57-E80, F83-P105 | BLIMPS_PRINTS |
| | | | PRECURSOR SIGNAL PROTEIN LECTIN REG LITHOSTATHINE REGENERATING | BLAST_PRODOM |
| | | | INFLAMMATORY RESPONSE ACUTE PD149843: Q21-L62 | |
| | | | C-TYPE LECTIN DM00035\|P48304\|29-163: L29-R110 | BLAST_DOMO |
| | | | C-TYPE LECTIN DM00035\|S34591\|29-163: L29-R110 | BLAST_DOMO |
| | | | C-TYPE LECTIN DM00035\|A45751\|29-163: L29-R110 | BLAST_DOMO |
| | | | C-TYPE LECTIN DM00035\|P05451\|29-163: L29-R110 | BLAST_DOMO |
| | | | Potential Phosphorylation Sites: S25 S35 S91 S92 S109 S121 S126 T56 T77 | MOTIFS |

TABLE 4

| Polynucleotide | |
|---|---|
| SEQ ID NO:/ | |
| Incyte ID/Sequence | |
| Length | Sequence Fragments |
| 21/7521032CB1/ | 1-272, 1-284, 1-285, 1-302, 1-367, 1-369, 1-374, 1-467, 6-285, 282-1137, 282-1143, |
| 1143 | 317-1143, 422-1143, 846-1143, 853-1143, 857-1142, 857-1143 |
| 22/2936048CB1/ | 1-144, 1-216, 1-259, 1-426, 1-918, 2-311, 7-249, 8-310, 13-292, 184-466, 562-1163, 590-1261, |
| 2591 | 807-1076, 852-1086, 896-1626, 928-1215, 936-1232, 936-1234, 1074-1316, 1123-1693, 1174-1783, 1267-1658, |
| | 1292-1580, 1295-1782, 1301-1609, 1330-1949, 1354-1619, 1554-2338, |
| | 1740-2239, 1844-2103, 1927-2470, 1954-2341, 1954-2386, 2004-2470, 2008-2470, 2031-2470, 2038-2314, |
| | 2038-2470, 2047-2470, 2048-2470, 2050-2429, 2053-2470, 2083-2470, 2113-2177, 2122-2383, 2149-2389, |
| | 2149-2408, 2172-2209, 2182-2470, 2193-2286, 2211-2470, 2219-2340, 2313-2470, 2334-2591, 2464-2585, |
| | 2465-2491, 2469-2585, 2490-2585, 2518-2585, 2565-2585 |
| 23/7521726CB1/ | 1-442, 1-907, 2-1122, 125-1123 |
| 1123 | |
| 24/7523383CB1/ | 1-74, 1-606, 1-753, 2-734, 13-780 |
| 780 | |
| 25/7522027CB1/ | 1-963, 2-722, 642-1418 |
| 1418 | |
| 26/7524406CB1/ | 1-753, 1-877, 2-669, 2-676, 2-718, 2-2075, 423-1416, 634-1397, 660-1588, 682-1495, 1316-2075, |
| 2076 | 1321-2076, 1329-2075 |
| 27/7524922CB1/ | 1-660, 2-660, 2-783, 98-199, 98-253, 98-322, 98-406, 167-460, 167-475, 236-460, 305-460, |
| 783 | 374-475 |
| 28/7524936CB1/ | 1-695, 2-540, 179-264, 179-280, 179-332, 179-333, 179-334, 179-401, 179-402, 179-470, 179-471, |
| 1115 | 179-556, 179-625, 210-403, 248-609, 248-692, 268-472, 317-678, 317-694, 336-556, 386-679, 405-625, |
| | 455-679, 474-1115, 524-679, 544-694, 593-694, 613-694 |
| 29/7512039CB1/ | 1-329, 1-521, 1-528, 60-519, 60-528 |
| 528 | |
| 30/7512576CB1/ | 1-474, 13-319, 16-613, 18-306, 19-623, 21-470, 114-366, 205-709, 374-621, 374-624, 477-727, |
| 1365 | 529-791, 668-1108, 678-917, 715-1055, 719-1337, 723-1055, 730-956, 732-1365, 746-1055, 755-1355, |
| | 777-1055, 786-1345, 824-1095, 839-1108, 840-1117, 856-1125, 877-1103 |
| 31/7514864CB1/ | 1-870, 2-1342, 532-1343 |
| 1343 | |
| 32/8266965CB1/ | 1-651, 2-457, 2-465, 42-529, 53-783, 105-371, 105-398, 105-549, 105-624, 105-632, 105-661, |
| 1840 | 105-682, 105-693, 105-708, 105-924, 106-446, |
| | 112-623, 114-200, 129-741, 207-465, 226-519, 230-639, 237-519, 243-419, 252-572, 269-537, 270-723, |
| | 276-995, 291-779, 292-603, 292-618, 342-478, 357-572, 383-808, 384-642, 384-914, 384-1039, 410-683, |
| | 410-764, 410-910, 416-685, 418-799, 419-584, 419-691, 431-1054, 455-731, 463-1184, 481-1187, 489-957, |
| | 495-670, 510-967, 522-1006, 528-1093, 534-1028, 540-1179, 542-1121, 543-1044, 546-1205, 548-925, |
| | 553-1257, 554-1123, 575-1012, 577-855, 593-921, 593-1177, 597-1243, 599-1057, 623-1111, 624-1176, |
| | 636-1261, 659-904, 663-1201, 669-1168, 672-1142, 674-1339, 678-1221, 692-897, 698-1308, 701-1136, |
| | 725-1249, 725-1308, 734-1053, 738-1221, 738-1264, 758-1323, 760-955, 762-1047, 764-1016, 764-1271, |
| | 764-1297, 770-1423, 778-1253, 782-1388, 788-1068, 795-1364, 799-1054, 799-1376, 806-1071, 806-1237, |
| | 806-1384, 808-1276, 814-1138, 820-1302, 827-1313, 831-1327, 843-1404, 844-1003, 845-1663, |
| | 847-1427, 860-1129, 864-1389, 864-1507, 877-1521, 880-1277, 890-1356, 910-1393, 919-1400, 931-1479, |
| | 939-1185, 947-1485, 969-1486, 973-1404, 983-1213, 989-1199, 994-1271, 1013-1614, 1029-1719, 1043-1490, |
| | 1050-1733, 1062-1454, 1065-1562, 1070-1527, 1077-1339, 1077-1486, 1082-1286, 1102-1373, 1119-1389, |
| | 1124-1573, 1124-1602, 1146-1400, 1149-1630, 1180-1832, 1182-1840, 1186-1516, 1190-1566, 1190-1691, |
| | 1192-1705, 1193-1674, 1193-1726, 1200-1464, 1228-1523, 1237-1731, 1237-1745, 1238-1499, 1248-1840, |
| | 1249-1422, 1250-1840, 1253-1719, 1256-1682, 1259-1767, 1261-1691, 1279-1840, 1292-1840, 1338-1593, |
| | 1346-1764, 1347-1707, 1353-1609, 1353-1840, 1374-1820, 1374-1840, 1379-1840, 1397-1840, 1399-1840, |
| | 1402-1737, 1411-1840, 1423-1609, 1425-1840, 1435-1646, 1436-1734, 1442-1840, 1450-1711, 1451-1840, |
| | 1452-1798, 1457-1686, 1463-1519, 1473-1691, 1477-1840, 1486-1840, 1512-1839, 1512-1840, 1519-1840, |
| | 1522-1668, 1529-1741, 1529-1788, 1531-1840, 1536-1840, 1545-1840, 1549-1840, 1600-1840, 1603-1840, |
| | 1621-1840, 1622-1833, 1663-1840 |
| 33/7515124CB1/ | 1-522, 1-523, 2-520, 60-523 |
| 523 | |
| 34/7514570CB1/ | 1-393, 2-393, 2-924 |
| 924 | |
| 35/7515114CB1/ | 1-831, 12-1345, 604-1346 |
| 1346 | |
| 36/7515136CB1/ | 1-811, 1-914, 12-1378, 600-1379 |
| 1379 | |
| 37/7515308CB1/ | 1-673, 1-780, 1-786, 1-942, 3-835, 158-999 |
| 999 | |
| 38/7516738CB1/ | 1-362, 1-777, 2-745, 212-1072 |
| 1072 | |
| 39/7518619CB1/ | 1-686, 1-872, 2-871 |
| 872 | |
| 40/7513061CB1/ | 1-169, 1-300, 1-359, 1-410, 1-414, 1-415, 1-418, 1-421, 1-426, 1-427, 1-429, 1-432, |
| 864 | 1-435, 1-438, 1-442, 1-446, 1-455, 1-459, 1-464, 1-499, 1-528, 3-836, 4-326, 13-490, 37-505, |
| | 85-548, 104-572, 113-591, 114-598, 135-604, 150-560, 150-604, 179-565, 237-562, 240-428, 249-520, |
| | 251-531, 251-549, 254-353, 254-452, 254-526, 254-541, 254-544, 254-552, 257-594, 277-535, 277-539, |
| | 283-560, 283-578, 298-594, 301-414, 305-516, 309-547, 309-575, 314-531, 319-543, 325-562, 336-602, |
| | 357-594, 461-726, 463-578, 490-712, 603-807, 603-831, 603-850, 603-851, 603-863, 604-806, 611-864, |
| | 612-809, 623-864, 624-841, 624-864, 636-864, 638-838, 641-840, 646-864, 648-855, 648-860, 667-857, |
| | 679-858, 705-850, 711-857, 711-864, 717-864, 724-864, 744-831, 749-864, 751-864, 752-864 |

TABLE 5

| Polynucleotide SEQ | | |
|---|---|---|
| ID NO: | Incyte Project ID: | Representative Library |
| 22 | 2936048CB1 | UTRSTMR02 |
| 29 | 7512039CB1 | BRAXTDR17 |
| 30 | 7512576CB1 | PROSTMT07 |
| 32 | 8266965CB1 | BRAFNON02 |
| 40 | 7513061CB1 | ISLTNOT01 |

TABLE 6

| Library | Vector | Library Description |
|---|---|---|
| BRAFNON02 | pINCY | This normalized frontal cortex tissue library was constructed from 10.6 million independent clones from |
| | | a frontal cortex tissue library. Starting RNA was made from superior frontal cortex tissue removed from a 35-year-old |
| | | Caucasian male who died from cardiac failure. Pathology indicated moderate leptomeningeal fibrosis and multiple |
| | | microinfarctions of the cerebral neocortex. Grossly, the brain regions examined and cranial nerves were unremarkable. |
| | | No atherosclerosis of the major vessels was noted. Microscopically, the cerebral hemisphere revealed moderate |
| | | fibrosis of the leptomeninges with focal calcifications. There was evidence of shrunken and slightly eosinophilic |
| | | pyramidal neurons throughout the cerebral hemispheres. There were also multiple small microscopic areas of |
| | | cavitation with surrounding gliosis scattered throughout the cerebral cortex. Patient history included dilated |
| | | cardiomyopathy, congestive heart failure, cardiomegaly, and an enlarged spleen and liver. Patient medications |
| | | included simethicone, Lasix, Digoxin, Colace, Zantac, captopril, and Vasotec. The library was normalized in |
| | | two rounds using conditions adapted from Soares et al., PNAS (1994) 91: 9228 and Bonaldo et al., Genome |
| | | Research (1996) 6: 791, except that a significantly longer (48 hours/round) reannealing hybridization was used. |
| BRAXTDR17 | PCDNA2.1 | This random primed library was constructed using RNA isolated from temporal neocortex |
| | | tissue removed from a 55-year-old Caucasian female who died from cholangiocarcinoma. Pathology indicated mild |
| | | meningeal fibrosis predominately over the convexities, scattered axonal spheroids in the white matter of the cingulate |
| | | cortex and the thalamus, and a few scattered neurofibrillary tangles in the entorhinal cortex and the periaqueductal |
| | | gray region. Pathology for the associated tumor tissue indicated well-differentiated cholangiocarcinoma of the liver |
| | | with residual or relapsed tumor. Patient history included cholangiocarcinoma, post-operative Budd-Chiari syndrome, |
| | | biliary ascites, hydrothorax, dehydration, malnutrition, oliguria and acute renal failure. Previous surgeries included |
| | | cholecystectomy and resection of 85% of the liver. |
| ISLTNOT01 | pINCY | Library was constructed using RNA isolated from a pooled collection of pancreatic islet cells. |
| PROSTMT07 | pINCY | The library was constructed using RNA isolated from diseased prostate tissue removed |
| | | from a 73-year-old Caucasian male during radical prostatectomy, closed prostatic biopsy, and regional lymph |
| | | node excision. Pathology indicated adenofibromatous hyperplasia. Pathology for the associated tumor tissue |
| | | indicated adenocarcinoma, Gleason 3 + 3, involving the left side peripherally and anteriorly. The tumor |
| | | perforated the capsule to involve periprostatic tissue and anterior surgical margin on the left. The patient |
| | | presented with elevated prostate-specific antigen. Patient history included bladder cancer, speech disturbance |
| | | and acquired spondylolisthesis. Family history included benign hypertension and cerebrovascular disease. |
| UTRSTMR02 | PCDNA2.1 | This random primed library was constructed using pooled cDNA from two different donors. |
| | | cDNA was generated using mRNA isolated from endometrial tissue removed from a 32-year-old female (donor A) |
| | | and using mRNA isolated from myometrium removed from a 45-year-old female (donor B) during vaginal hysterectomy |
| | | and bilateral salpingo-oophorectomy. In donor A, pathology indicated the endometrium was secretory phase. |
| | | The cervix showed severe dysplasia (CIN III) focally involving the squamocolumnar junction at the 1, 6 and |
| | | 7 o'clock positions. Mild koilocytotic dysplasia was also identified within the cervix. In donor B, pathology |
| | | for the matched tumor tissue indicated multiple (23) subserosal, intramural, and submucosal leiomyomata. |
| | | Patient history included stress incontinence, extrinsic asthma without status asthmaticus and normal delivery |
| | | in donor B. Family history included cerebrovascular disease, depression, and atherosclerotic coronary artery |
| | | disease in donor B. |

TABLE 7

| Program | Description | Reference | Parameter Threshold |
|---|---|---|---|
| ABI | A program that removes vector sequences and masks | Applied Biosystems, Foster City, CA. | |
| FACTURA | ambiguous bases in nucleic acid sequences. | | |
| ABI/ | A Fast Data Finder useful in comparing and | Applied Biosystems, Foster City, CA; | Mismatch <50% |
| PARACEL | annotating amino acid or nucleic acid sequences. | Paracel Inc., Pasadena, CA. | |
| FDF | | | |
| ABI | A program that assembles nucleic acid sequences. | Applied Biosystems, Foster City, CA. | |
| AutoAssembler | | | |
| BLAST | A Basic Local Alignment Search Tool useful in | Altschul, S. F. et al. (1990) J. Mol. Biol. | ESTs: Probability |
| | sequence similarity search for amino acid and nucleic | 215: 403-410; Altschul, S. F. et al. (1997) | value = 1.0E−8 |
| | acid sequences. BLAST includes five functions: | Nucleic Acids Res. 25: 3389-3402. | or less; Full Length |
| | blastp, blastn, blastx, tblastn, and tblastx. | | sequences: |
| | | | Probability value = |
| | | | 1.0E−10 or less |
| FASTA | A Pearson and Lipman algorithm that searches for | Pearson, W. R. and D. J. Lipman (1988) Proc. | ESTs: fasta E |
| | similarity between a query sequence and a group of | Natl. Acad Sci. USA 85: 2444-2448; Pearson, W. R. | value = 1.06E−6; |
| | sequences of the same type. FASTA comprises as | (1990) Methods Enzymol. 183: 63-98; | Assembled ESTs: |
| | least five functions: fasta, tfasta, fastx, tfastx, and | and Smith, T. F. and M. S. Waterman (1981) | fasta Identity = |
| | ssearch. | Adv. Appl. Math. 2: 482-489. | 95% or greater and |
| | | | Match length = |
| | | | 200 bases or greater; |
| | | | fastx E value = |
| | | | 1.0E−8 or less; |
| | | | Full Length sequences: |
| | | | fastx score = 100 |
| | | | or greater |
| BLIMPS | A BLocks IMProved Searcher that matches a | Henikoff, S. and J. G. Henikoff (1991) | Probability value = |
| | sequence against those in BLOCKS, PRINTS, | Nucleic Acids Res. 19: 6565-6572; Henikoff, J. G. | 1.0E−3 or less |
| | DOMO, PRODOM, and PFAM databases to search | and S. Henikoff (1996) Methods | |
| | for gene families, sequence homology, and structural | Enzymol. 266: 88-105; and Attwood, T. K. et | |
| | fingerprint regions. | al. (1997) J. Chem. Inf. Comput. Sci. 37: 417- | |
| HMMER | An algorithm for searching a query sequence against | Krogh, A. et al. (1994) J. Mol. Biol. | PFAM, INCY, |
| | hidden Markov model (HMM)-based databases of | 235: 1501-1531; Sonnhammer, E. L. L. et al. | SMART or |
| | protein family consensus sequences, such as PFAM, | (1988) Nucleic Acids Res. 26: 320-322; | TIGRFAM hits: |
| | INCY, SMART and TIGRFAM. | Durbin, R. et al. (1998) Our World View, in | Probability value = |
| | | a Nutshell, Cambridge Univ. Press, pp. 1- | 1.0E−3 or less; |
| | | | Signal peptide hits: |
| | | | Score = 0 or greater |
| ProfileScan | An algorithm that searches for structural and | Gribskov, M. et al. (1988) CABIOS 4: 61-66; | Normalized quality |
| | sequence motifs in protein sequences that match | Gribskov, M. et al. (1989) Methods | score ≧ GCG |
| | sequence patterns defined in Prosite. | Enzymol. 183: 146-159; Bairoch, A. et al. | specified “HIGH” |
| | | (1997) Nucleic Acids Res. 25: 217-221. | value for that |
| | | | particular |
| | | | Prosite motif. |
| | | | Generally, score = |
| | | | 1.4-2.1. |
| Phred | A base-calling algorithm that examines automated | Ewing, B. et al. (1998) Genome Res. 8: 175-185; | |
| | sequencer traces with high sensitivity and probability. | Ewing, B. and P. Green (1998) Genome | |
| | | Res. 8: 186-194. | |
| Phrap | A Phils Revised Assembly Program including | Smith, T. F. and M. S. Waterman (1981) Adv. | Score = 120 |
| | SWAT and CrossMatch, programs based on efficient | Appl. Math. 2: 482-489; Smith, T. F. and | or greater; |
| | implementation of the Smith-Waterman algorithm, | M. S. Waterman (1981) J. Mol. Biol. 147: 195-197; | Match length = 56 |
| | useful in searching sequence homology and | and Green, P., University of | or greater |
| | assembling DNA sequences. | Washington, Seattle, WA. | |
| Consed | A graphical tool for viewing and editing Phrap | Gordon, D. et al. (1998) Genome Res. 8: 195-202. | |
| | assemblies. | | |
| SPScan | A weight matrix analysis program that scans protein | Nielson, H. et al. (1997) Protein Engineering | Score = 3.5 |
| | sequences for the presence of secretory signal | 10: 1-6; Claverie, J. M. and S. Audic (1997) | or greater |
| | peptides. | CABIOS 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos | |
| | determine orientation. | (1996) Protein Sci. 5: 363-371. | |
| TMHMMER | A program that uses a hidden Markov model (HMM) | Sonnhammer, E. L. et al. (1998) Proc. Sixth | |
| | to delineate transmembrane segments on protein | Intl. Conf. On Intelligent Systems for Mol. | |
| | sequences and determine orientation. | Biol., Glasgow et al., eds., The Am. Assoc. | |
| | | for Artificial Intelligence (AAAI) Press, | |
| | | Menlo Park, CA, and MIT Press, Cambridge, | |
| | | MA, pp. 175-182. | |
| Motifs | A program that searches amino acid sequences for | Bairoch, A. et al. (1997) Nucleic Acids Res. | |
| | patterns that matched those defined in Prosite. | 25: 217-221; Wisconsin Package Program | |
| | | Manual, version 9, page M51-59, Genetics | |
| | | Computer Group, Madison, WI. | |

TABLE 8

| | | | | | | | | | | | | | His- |
|---|---|---|---|---|---|---|---|---|---|---|---|---|---|
| | | | | | | | | | | | | | panic |
| SEQ | | | | | | | | | | Caucasian | African | Asian | Allele 1 |
| ID | | | | EST | CB1 | EST | | | Amino | Allele 1 | Allele 1 | Allele 1 | freq- |
| NO: | PID | EST ID | SNP ID | SNP | SNP | Allele | Allele 1 | Allele 2 | Acid | frequency | frequency | frequency | uency |
| 22 | 2936048 | 5338178H1 | SNP00058488 | 99 | 2412 | C | C | T | noncoding | n/a | n/a | n/a | n/a |
| 26 | 7524406 | 2749757H1 | SNP00121108 | 213 | 1387 | T | T | C | Y452 | n/a | n/a | n/a | n/a |
| 26 | 7524406 | 4739562R7 | SNP00033062 | 268 | 1933 | C | C | T | noncoding | n/a | n/a | n/a | n/a |
| 29 | 7512039 | 2875524H1 | SNP00131200 | 2 | 57 | G | A | G | noncoding | n/a | n/a | n/a | n/a |
| 31 | 7514864 | 1282428H1 | SNP00075286 | 180 | 572 | C | C | T | T186 | n/d | n/d | n/d | n/d |
| 31 | 7514864 | 1282428H1 | SNP00106459 | 90 | 482 | C | C | G | T156 | n/a | n/a | n/a | n/a |
| 31 | 7514864 | 1299002H1 | SNP00106460 | 204 | 693 | G | G | C | L226 | n/d | n/a | n/a | n/a |
| 31 | 7514864 | 1332279H1 | SNP00009699 | 27 | 975 | G | A | G | T320 | n/a | n/a | n/a | n/a |
| 31 | 7514864 | 1332279H1 | SNP00097916 | 45 | 993 | C | C | T | G326 | n/a | n/a | n/a | n/a |
| 31 | 7514864 | 1365956R1 | SNP00009700 | 32 | 1224 | C | C | T | Y403 | n/a | n/a | n/a | n/a |
| 31 | 7514864 | 1401460H1 | SNP00053363 | 87 | 105 | G | G | A | L30 | n/d | n/a | n/a | n/a |
| 31 | 7514864 | 1483017H1 | SNP00075287 | 63 | 855 | G | G | A | Q280 | n/a | n/a | n/a | n/a |
| 31 | 7514864 | 2113488H1 | SNP00065694 | 65 | 83 | C | C | T | A23 | n/d | n/d | n/d | n/d |
| 31 | 7514864 | 2878691F6 | SNP00065694 | 65 | 84 | C | C | T | A23 | n/d | n/d | n/d | n/d |
| 31 | 7514864 | 2992902T6 | SNP00009700 | 288 | 1225 | C | C | T | R404 | n/a | n/a | n/a | n/a |
| 31 | 7514864 | 3908734H1 | SNP00106459 | 211 | 484 | C | C | G | L157 | n/a | n/a | n/a | n/a |
| 31 | 7514864 | 5594636H1 | SNP00134605 | 86 | 374 | A | A | C | D120 | n/a | n/a | n/a | n/a |
| 31 | 7514864 | 6182410H1 | SNP00053363 | 106 | 107 | G | G | A | R31 | n/d | n/a | n/a | n/a |
| 31 | 7514864 | 7361170H1 | SNP00075287 | 111 | 856 | G | G | A | A281 | n/a | n/a | n/a | n/a |
| 31 | 7514864 | 7361170H1 | SNP00097916 | 249 | 994 | C | C | T | L327 | n/a | n/a | n/a | n/a |
| 31 | 7514864 | 7715155J1 | SNP00075286 | 157 | 550 | C | C | T | P179 | n/d | n/d | n/d | n/d |
| 31 | 7514864 | 7715155J1 | SNP00075287 | 441 | 833 | G | G | A | R273 | n/a | n/a | n/a | n/a |
| 31 | 7514864 | 7715155J1 | SNP00106459 | 67 | 460 | C | C | G | P149 | n/a | n/a | n/a | n/a |
| 31 | 7514864 | 7715155J1 | SNP00106460 | 278 | 671 | G | G | C | R219 | n/d | n/a | n/a | n/a |
| 32 | 8266965 | 2074442H1 | SNP00019661 | 135 | 1706 | C | C | G | noncoding | n/a | n/a | n/a | n/a |
| 32 | 8266965 | 924738T6 | SNP00019661 | 16 | 1783 | C | C | G | noncoding | n/a | n/a | n/a | n/a |
| 34 | 7514570 | 2837168F6 | SNP00099432 | 62 | 58 | C | C | G | N19 | n/a | n/a | n/a | n/a |
| 37 | 7515308 | 2578987T6 | SNP00018529 | 131 | 887 | G | T | G | noncoding | n/a | n/a | n/a | n/a |
| 37 | 7515308 | 3821990T8 | SNP00018529 | 19 | 886 | T | T | G | noncoding | n/a | n/a | n/a | n/a |
| 38 | 7516738 | 1507546H1 | SNP00012041 | 124 | 136 | T | C | T | M31 | 0.55 | n/a | n/a | n/a |
| 38 | 7516738 | 1507546H1 | SNP00044789 | 112 | 124 | C | C | T | S27 | n/d | n/d | n/d | n/d |
| 38 | 7516738 | 1507582T6 | SNP00012042 | 273 | 842 | T | T | C | V266 | n/a | n/a | n/a | n/a |
| 38 | 7516738 | 1508412H1 | SNP00012042 | 133 | 845 | T | T | C | A267 | n/a | n/a | n/a | n/a |
| 38 | 7516738 | 1607858T6 | SNP00012042 | 231 | 861 | T | T | C | F273 | n/a | n/a | n/a | n/a |
| 38 | 7516738 | 1986360T6 | SNP00012042 | 273 | 844 | T | T | C | V267 | n/a | n/a | n/a | n/a |
| 38 | 7516738 | 2219515H1 | SNP00044790 | 123 | 465 | G | A | G | A141 | 0.44 | n/a | n/a | n/a |
| 38 | 7516738 | 2353892T6 | SNP00012042 | 253 | 847 | T | T | C | M268 | n/a | n/a | n/a | n/a |
| 38 | 7516738 | 3905387H1 | SNP00012041 | 111 | 138 | C | C | T | P32 | 0.55 | n/a | n/a | n/a |
| 38 | 7516738 | 3905387H1 | SNP00044789 | 99 | 126 | C | C | T | H28 | n/d | n/d | n/d | n/d |
| 39 | 7518619 | 1237113F6 | SNP00093216 | 74 | 196 | T | T | C | S55 | n/a | n/a | n/a | n/a |
| 39 | 7518619 | 1237113F6 | SNP00111774 | 239 | 362 | A | A | C | K111 | n/a | n/a | n/a | n/a |
| 39 | 7518619 | 1237113T6 | SNP00026831 | 40 | 772 | G | G | A | K247 | 0.87 | 0.99 | 0.6 | 0.59 |
| 39 | 7518619 | 1237113T6 | SNP00050656 | 398 | 414 | C | C | G | S128 | 0.66 | 0.66 | 0.61 | 0.74 |
| 39 | 7518619 | 3068815F6 | SNP00111774 | 483 | 361 | A | A | C | L110 | n/a | n/a | n/a | n/a |
| 39 | 7518619 | 5395205T1 | SNP00026831 | 106 | 764 | A | G | A | K245 | 0.87 | 0.99 | 0.6 | 0.59 |
| 39 | 7518619 | 5395205T1 | SNP00050656 | 464 | 406 | C | C | G | S125 | 0.66 | 0.66 | 0.61 | 0.74 |
| 39 | 7518619 | 6212102H1 | SNP00054454 | 148 | 406 | C | C | G | S125 | 0.75 | n/a | n/a | n/a |
| 39 | 7518619 | 6492013H1 | SNP00068607 | 381 | 224 | T | C | T | Y65 | n/d | n/d | n/d | n/d |
| 40 | 7513061 | 2078146H1 | SNP00149880 | 19 | 425 | C | T | C | H48 | n/a | n/a | n/a | n/a |
| 40 | 7513061 | 2078441H1 | SNP00054200 | 221 | 596 | C | C | T | P105 | n/a | n/a | n/a | n/a |

1.-95. (canceled)

96. An isolated polypeptide selected from the group consisting of:
(a) a polypeptide comprising an amino acid sequence of SEQ ID NO: 5;
(b) a biologically active fragment of the polypeptide of (a); and
(c) an immunogenic fragment of the polypeptide of (a).

97. An isolated polypeptide of claim 96 consisting of the polypeptide of (a).

98. An isolated polypeptide of claim 96 consisting of a biologically active fragment of the polypeptide of (a).

99. An isolated polypeptide of claim 96 consisting of an immunogenic fragment of the polypeptide of (a).

100. An isolated polypeptide of claim 96 encoded by a polynucleotide selected from the group consisting of:
(i) a polynucleotide comprising a polynucleotide sequence of SEQ ID NO: 25;
(ii) a polynucleotide comprising a naturally occurring polynucleotide sequence at least 90% identical to SEQ ID NO: 25;
(iii) a polynucleotide comprising a portion of the polynucleotide sequence of SEQ ID NO: 53 that specifically identifies SEQ ID NO: 25.
(iv) a polynucleotide comprising a polynucleotide complementary to the polynucleotide of (i), (ii), or (iii);
(v) an RNA equivalent of the polynucleotide of (i), (ii), (iii) or (iv);
(vi) a polynucleotide of (i), (ii) or (iii) further comprising a promoter sequence operably linked to said polynucleotide of (i), (ii) or (iii).

101. An isolated polypeptide of claim 96 produced recombinantly.

102. An isolated polypeptide of claim 100 produced by culturing a cell transformed with a polynucleotide of (iv) under conditions suitable for expression of the polypeptide, and recovering the polypeptide so expressed.

103. An isolated antibody that specifically binds to a polypeptide of claim 96.

104. An isolated antibody of claim 103, wherein said antibody is selected from the group consisting of a polyclonal antibody, a monoclonal antibody, a chimeric antibody, a single chain antibody, a Fab fragment, a F(ab′)₂ fragment, and a humanized antibody.

105. An isolated antibody of claim 103, wherein said antibody is selected by screening a recombinant immunoglobulin library.

106. An isolated antibody of claim 103, wherein said antibody is selected by screening a Fab expression library.

107. An isolated antibody that specifically binds to a polypeptide of claim 100.

108. An isolated antibody of claim 107, wherein said antibody is selected from the group consisting of a polyclonal antibody, a monoclonal antibody, a chimeric antibody, a single chain antibody, a Fab fragment, a F(ab′)₂ fragment, and a humanized antibody.

109. An isolated antibody of claim 107, wherein said antibody is selected by screening a recombinant immunoglobulin library.

110. An isolated antibody of claim 107, wherein said antibody is selected by screening a Fab expression library.

111. A method of detecting a polypeptide of interest in a sample, comprising:
incubating the sample with an antibody that specifically binds to a polypeptide of claim 96 under conditions suitable for binding of the antibody to the polypeptide of interest if present in the sample; and
detecting biding of the polypeptide of interest to the antibody, wherein binding indicates the presence or amount of the polypeptide of interest in the sample.

112. A method of claim 111, wherein the sample is a body fluid sample from a human.

113. An isolated polynucleotide selected from the group consisting of:
(i) a polynucleotide comprising a polynucleotide sequence of SEQ ID NO: 25;
(ii) a polynucleotide comprising a naturally occurring polynucleotide sequence at least 90% identical to SEQ ID NO: 25;
(iii) a polynucleotide comprising a portion of the polynucleotide sequence of SEQ ID NO: 53 that specifically identifies SEQ ID NO: 25.
(iv) a polynucleotide comprising a polynucleotide complementary to the polynucleotide of (i), (ii), or (iii);
(v) an RNA equivalent of the polynucleotide of (i), (ii), (iii) or (iv);
(vi) a polynucleotide of (i), (ii) or (iii) further comprising a promoter sequence operably linked to said polynucleotide of (i), (ii) or (iii).


SEQUENCE LISTING (40 sequences)

SEQ ID NO: 1 - protein, 108 aa, Homo sapiens
  Incyte ID NO 7521032CD1
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPGKDPSKV

SEQ ID NO: 2 - protein, 622 aa, Homo sapiens [sequence omitted]

SEQ ID NO: 3 - protein, 210 aa, Homo sapiens
  Incyte ID NO 7521726CD1
MAGCVPLLQGLVLVLALHRVEPSVFLPASKANDVLVRWKRAGSYLLEELFEGNLEKECYEETCVYEEAREVFENEVVTDEFWRRYKGGSPCISQPCLHNGSCQDSIWGYTCTCSPGYEGSNCELAKNECHPERTDGCQHFCLPGQESYTCSCAQGYRLGEDHKQCVPHDQCACGVLTSEKRAPDLQDLPWQNEPRPADDQDNARPCAHAV

SEQ ID NO: 4 - protein, 248 aa, Homo sapiens
  Incyte ID NO 7523383CD1
MAKDFQDIQQLSSEENDHPFHQGAQLQAELRSLKEAFSNFSSSTLTEVQAISTHGGSVGDKITSLGAKLEKQQQDLKADHDALLFHLKHFPVDLRFVACQMELLHSNGSQRTCCPVNWVEHQGSCYWFSHSGKAWAEAEKYCQLENAHLVVINSWEEQKFIVQHTNPFNTWIGLTDSDGSWKWVDGTDYRHNYKNWAVTQPDNWHGHELGGSEDCVEVQPDGRWNDDFCLQVYRWVCGKRRNATGEVA

SEQ ID NO: 5 - protein, 97 aa, Homo sapiens
  Incyte ID NO 7522027CD1
MAGCVPLLQGLVLVLALHRVEPSVFLPASKANDVLVRWKRAGSYLLEELFEGNLEKECYEEICVYEEAREVFENEVVTDEFWRRYKGKWFPSSPQKY

SEQ ID NO: 6 - protein, 479 aa, Homo sapiens
  Incyte ID NO 7524406CD1
MGRIGISCLFPASWHFSISPVGCPRILNTNLRQIMVISVLAAAVSLLYFSVVIIRNKYGRLTRDKKFQRYLARVTDIEATDTNNPNVSYGIVVDCGSSGSRVFVYCWPRHNGNPHDLLDIRQMRDKNRKPVVMKIKPGISEFATSPEKVSDYISPLLNFAAEHVPRAKHKETPLYILCTAGMRILPESQQKAILEDLLTDIPVHFDFLFSDSHAEVISGKQEGVYAWIGINFVLGRFEHIEDDDEAVVEVNIPGSESSEAIVRKRTAGILDMGGVSTQIAYEVPKTEEVAKNLLAEFNLGCDVHQTEHVYRVYVATFLGFGGNAARQRYEDRIFANTIQKNRLLGKQTGLTPDMPYLDPCLPLDIKDEIQQNGQTIYLRGTGDFDLCRETIQPFMNKTNETQTSLNGVYQPPIHFQNSEFYGFSEFYYCTEDVLRMGGDYNAAKFTKAAKDYCATKWSILRERFDRGLYASHADLHRLK

SEQ ID NO: 7 - protein, 222 aa, Homo sapiens
  Incyte ID NO 7524922CD1
MSDSKEPRVQQLGLLVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTQLKAAVGELPDQSKQQQIYQELTDLKTAFERLCRHCPKDWTFFQGNCYFMSNSQRNWHNSVTACQEVRAQLVVIKTAEEQLPAVLEQWRTQQ

SEQ ID NO: 8 - protein, 370 aa, Homo sapiens
  Incyte ID NO 7524936CD1
MSDSKEPRVQQLGLLEDPTTSGIRLFPRDFQFQQIHGHKSSTVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAPRFRDE

SEQ ID NO: 9 - protein, 77 aa, Homo sapiens
  Incyte ID NO 7512039CD1
MNSSKSSETQCTERGCFSSQMFLWTVAGIPILFLSACFITRCVVTFRIFQTCDEKKFQLPENFTELSCYNYGSASGM

SEQ ID NO: 10 - protein, 415 aa, Homo sapiens
  Incyte ID NO 7512576CD1
MPAVSGPGPLFCLLLLLLDPHSPETGCPPLRRFEYKLSFKGPRLALPGAGIPFWSHHGDAILGLEEVRLTPSMRNRSGAVWSRASVPFSAWEVEVQMRVTGLGRRGAQGMAVWYTRGRGHVGSVLGGLASWDGIGIFFDSPAEDTQDSPAIRVLASDGHIPSEQPGDGASQGLGSCHWDFRNRPHPFRARITYWGQRLRMSLNSGLTPSDPDDHDVLSFLTFSLSEPSPEVPPQPFLEMQQLRLARQLEGLWARLGLGTREDVTPKSDSEAQGEGERLFDLEETLGRHRRILQALRGLSKQLAQAERQWKKQLGPPGQARPDGGWALDASCQIPSTPGRGGHLSMSLNKDSAKVGALLHGQWTLLQALQEMSRQELNKSLQECLSTGSLPLGPAPHTPRALGILMRQPLPASMPA

SEQ ID NO: 11 - protein, 441 aa, Homo sapiens
  Incyte ID NO 7514864CD1
MAAAMPLALLVLLLLGPGGWCLAEPPRDSLREELVITPLPSGDVAATFQFRTRWDSELQREGGLSVLLKADRLFHTSYHSQAVHIRPVCRNARCTSISWELRQTLSVVFDAFIAGQGKKDWSLFRMFSRTLTEPCPLASESRVYVDITTYNQDNETLEVHPPPTTTYQDVILGTRKTYAIYDLLDTAMINNSRNLNIQLKWKRPPENEAPPVPFLRAQRYVSGYGLQKGELSTLLYNTHPYRAFPVLLLDTVPWYLRLYVHTLTITSKGKENKPSYIHYQPAQDRLQPHLLEMLIQLPANSVTKVSIQFERALLKWTEYTPDPNHGFYVSPSVLSALVPSMVAAKPVDWEESPLFNSLFPVSDGSNYFVRLYTEPLLVNLPTPDFSMPYNVICLTCTVVAVCYGSFYNLLTRTFHIEEPRTGGLAKRLANLIRRARGVPPL

SEQ ID NO: 12 - protein, 283 aa, Homo sapiens
  Incyte ID NO 8266965CD1
MTQLKEAAIGVLVLSWYPPGMADDNGEPSDDLVPAILDTAHQYSIQVAFHIQPYKGRDDITVHDNIKYIIDTYGSHGAFYRYKNSMGKSLPLFYIYDSYLTSPEAWAHLLTPNGPHSIRNTPYDGVFIALLVEEGHTHDILAAGFDGMYTYFASNGFSFGSSHQNWKAVKNFCDANNLMFIPSVGPGYIDTSIRPWNNHNTRNRVNGKYYETALQAALTVRPEIVSITSFNEWHEGTQIEKAIPKKTPTRLYLDYLPHQPSLYLELTRRWAEHFIKEKEQWLM

SEQ ID NO: 13 - protein, 159 aa, Homo sapiens
  Incyte ID NO 7515124CD1
MSALWLLLGLLALMGVRASERLAEIDMPYLLKYQPMMQTIGQKYCMDPAVIAGVLSRKSPGDKILVNMGDRTSMVQDPGSQAPTSWISESQVSQTTEVLTTRIKEIQRRFPTWTPDQYLRGGLCAYSGGAGYVRSSQDLSCDFCNDVLARAKYLKRHGF

SEQ ID NO: 14 - protein, 154 aa, Homo sapiens
  Incyte ID NO 7514570CD1
MHDSNNVEKDITPSELPANPAIRANCHQEPSVCLQAACPESWIGFQRKCFYFSDDTKNWTSSQRFCDSQDADLAQVESFQELNFLLRYKGPSDHWIGLSREQGQPWKWINGTEWTRQFPILGAGECAYLNDKGASSARHYTERKWICSKSDIHV

SEQ ID NO: 15 - protein, 431 aa, Homo sapiens
  Incyte ID NO 7515114CD1
MPAVSGPGPLFCLLLLLLDPHSPETGCPPLRRFEYKLSFKGPRLALPGAGIPFWSHHGDAILGLEEVRLTPSMRNRSGAVWSRASVPFSAWEVEVQMRVTGLGRRGAQGMAVWYTRGRGHVGSVLGGLASWDGIGIFFDSPAEDTQDSPAIRVLASDGHIPSEQPGDGASQGLGSCHWDFRNRPHPFRARITYWGQRLRMSLNSGLTPSDPGEFCVDVGPLLLVPGGFFGVSAATGTLAGEDPTGQVPPQPFLEMQQLRLARQLEGLWARLGLGTREDVTPKSDSEAQGEGERLFDLEETLGRHRRILQALRGLSKQLAQAERQWKKQLGPPGQTRPDGGWALDASCQIPSTPGRGGHLSMSLNKDSAKVGALLHGQWTLLQALQEMSRQELNKSLQECLSTGSLPLGPAPHTPRALGILRRQPLPASMPA

SEQ ID NO: 16 - protein, 442 aa, Homo sapiens
  Incyte ID NO 7515136CD1
MPAVSGPGPLFCLLLLLLDPHSPETGCPPLRRFEYKLSFKGPRLALPGAGIPFWSHHGDAILGLEEVRLTPSMRNRSGAVWSRASVPFSAWEVEVQMRVTGLGRRGAQGMAVWYTRGRGHVGSVLGGLASWDGIGIFFDSPAEDTQDSPAIRVLASDGHIPSEQPGDGASQGLGSCHWDFRNRPHPFRARITYWGQRLRMSLNSGLTPSDPGEFCVDVGPLLLVPGGFFGVSAATGTLADDHDVLSFLTFSLSEPSPEVPPQPFLEMQQLRLARQLEGLWARLGLGTREDVTPKSDSEAQGEGERLFDLEETLGRHRRILQALRGLSKQLAQAERQWKKQLGPPGQARPDGGWALDASCQIPSTPGRGGHLSMSLNKDSAKVGALLHGQWTLLRALQEMRQELNKSLQECLSTGSLPLGPAPHTPRALGILRRQPLPASMPA

SEQ ID NO: 17 - protein, 198 aa, Homo sapiens
  Incyte ID NO 7515308CD1
MTSEITYAEVRFKNEFKSSGINTASSAVFFQKYSQLLEKKTTKELVHTTLECVKKNMPVEETAWSCCPKNWKSFSSNCYFISTESASWQDSEKDCARMEAHLLVINTQEEQDFIFQNLQEESAYFVGLSDPEGQRHWQWVDQTPYNESSAFWHPREPSDPNERCVVLNFRKSPKRWGWNDVNCLGPQRSVCEMMKIHL

SEQ ID NO: 18 - protein, 336 aa, Homo sapiens
  Incyte ID NO 7516738CD1
MLLFLLSALVLLTQPLGYLEAEMKTYSHRTMPSACTLVMCSSVESGLPGRDGRDGREGPRGEKGDPGLPGAAGQAGMPGQAGPVGPKGDNGSVGEPGPKGDTGPSGEVGAPGMQGSAGARGLAGPKGERGVPGERGVPGNAGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 19 - protein, 258 aa, Homo sapiens
  Incyte ID NO 7518619CD1
MMLSLNNLQNIIYNPVIPYVGTIPDQLDPGTLIVICGHVPSDADRFQVDLQNGSSVKPRADVAFHFNPRFKRAGCIVCNTLINEKWGREEITYDTPFKREKSFEIVIMVLKDKFQVPKSGTPQLSLPFAARLNTPMGPGRTVVVKGEVNANAKSFNVDLLAGKSKDIALHLNPRLNIKAFVRNSFLQESWGEEERNITSFPFSPGMYFEMIIYCDVREFKVAVNGVHSLEYKHRFKELSSIDTLEINGDIHLLEVRSW

SEQ ID NO: 20 - protein, 132 aa, Homo sapiens
  Incyte ID NO 7513061CD1
MAQTNSFFMLISSLMFLSLSQGQESQTELPNPRISCPEGTNAYRSYCYYFNEDPETWVDADLYCQNMNSGNLVSVLTQAEGAFVASLIKESSTDDSNVWIGLHDPKKDSRNGRMNLVRRSSPLFASSKTRGS

SEQ ID NO: 21 - DNA, 1143 bp, Homo sapiens
  Incyte ID NO 7521032CB1
gcctgtgggttgcagtaaaaagacaaggagggcctgagtgatatgacccttcagatagggaactcacagacggccagatgggaggtggagcagggacgtcattccactggccatttttcagtagcaatacacaatcttcatcagaaccagcattgttgggttcaccctcgttccagtttgtgtaggtcagtctatttcctgtcagatccacaaactgcccttctgtcttctcatcagtgatgcccaggaaggcttcctccttgatgagattctgaatggctccatgtccctgtttccatcactccctctccttctcctgagtatggtggcagcgtcttactcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctgggtcaccaggaccaaagggccaaaaaggagaccctggaaaaagtccgggtaaggaccccagcaaggtctgagctgacttcacccaggggttctgagaccttgagtatctgatggtgatagtagcctggctgcctcagaaagaaaagctctgcaaacagaaatggcacgtatcaaaaagtggctgaccttctctctgggcaaacaagttgggaacaagttcttcctgaccaatggtgaaataatgacctttgaaaaagtgaaggccttgtgtgtcaagttccaggcccctgtggccacccccaggaatgctgcagagaatggagccattcagaatctcatcaaggaggaagccttcctgggcatcactgatgagaagacagaagggcagtttgtggatctgacaggaaatagactgacctacacaaactggaacgagggtgaacccaacaatgctggttctgatgaagattgtgtattgctactgaaaaatggccagtggaatgacgtcccctgctccacctcccatctggccgtctgtgagttccctatctgaagggtcatatcactcaggccctccttgtctttttactgcaacccacaggca

SEQ ID NO: 22 - DNA, 2591 bp, Homo sapiens
  Incyte ID NO 2936048CB1
cgcggcgcgaccgcctcagtcacttcgcccagagacccggacctggtccgctggggagcaggcggccataaaccccctctctcccggttccctgacgccgcggcaggagctgttacaaacaccctgcggttggtctccgatgcccttcagtgaggtggggacgcctggaccctggtgagcgaaccccaagccaccccccaccccaactcagtgtcttcgccggcccccggcccgtacgcctgtctggtcgccatggctgaaaacacagagggggatctgaactccaacctgctccacgccccctaccacaccggggaccctcagctggacacggccatcgggcagtggctccgctgggataagaatcccaaaacaaaagagcagattgaaaacctgttacggaatgggatgaacaaggagctgcgagatcgtctttgttgccgaatgacttttgggactgcaggacttcgttctgccatgggggcagggttttgctatattaatgaccttacagtaatacagtcaacacaggggatgtacaaataccttgagagatgtttctcagacttcaagcagagaggctttgtggttgggtatgacactcggggtcaagtaactagcagctgcagcagccagaggcttgctaaactcactgctgcagtcttgctggccaaagatgttcctgtgtaccttttttcaagatatgttcctacaccttttgtaccatatgcagttcagaagctcaaagcagttgcaggtgtgatgattactgcctctcacaaccgcaaggaagacaatggatacaaggtttactgggaaactggtgctcagatcacatctcctcatgataaagaaattctaaaatgtatagaagaatgtgtggaaccctggaatggttcctggaatgataatttagtggataccagcccgctgaagagagaccctctgcaggacatttgcaggagatacatggaagatctgaaaaagatctgtttttacagggagttaaactcgaagaccaccttgaaatttgtgcacacatcttttcatggggtcggacatgactatgtgcagttggcttttaaagtgtttggttttaagcctccaattccagtaccagaacaaaaagatcctgatccagacttttctaccgttaaatgtccaaatcctgaagaaggagaatctgtgctggaactttccttgagactggcagagaaagaaaatgcccgggtagtgctagccacagatcctgatgcagacagactggcagcagcagaacttcaggagaatggttgttggaaagttttcacagggaatgagttggcagctttgtttggatggtggatgtttgattgctggaagaaaaataaatcaagaaatgctgatgtgaagaacgtttatatgttagccaccacagtctcttctaaaattctgaaggcaattgcacttaaagaaggatttcattttgaagaaacattaccaggttttaaatggattggaagtaggataatagacctcctggaaaatgggaaagaagtcctttttgcatttgaagagtctattggttttctctgtggaacttcagttttggataaagatggggtgagtgcagctgttgtggttgctgagatggcatcttacctggaaaccatgaatataacattgaaacagcaactggttaaggtttatgaaaaatatggttatcatatttcaaaaacttcctatttcttgtgttatgaaccacctaccatcaaaagtatatttgaaaggcttcgtaattttgattctccaaaagaatatccaaaattttgtggaacatttgctatattgcatgtacgggacattaccactggatatgacagtagccagcctaataagaaatcagtgctgcctgtgagtaaaaacagccaaatgattacatttacttttcaaaatggctgtgttgctacccttcggacaagtggaacagaaccaaagataaagtattatgcagagatgtgtgcgtcacctgaccagagtgacactgctttactggaggaagaactgaagaaactcattgatgctctgatagagaattttcttcagcctagtaagaatggactgatctggcgttctgtttaggggtacaccaatatgtcatgacactgtgtgggcatatggaacagagcaaccgagaactgcatacattgaaccttgtgttagcattctctctctatctcatctggccgagtatctttttccttttattttctttctttttggtcaactggctagactaaacaaagtataagtttgaaatgaaatggtctagagggaaatgattcaaattttttcataattcaaacaaaagttattacactaaaagtattatagtaacgtattgtccttccgttaacagaaacatcacaattcaaaagtgagttttcttattatagtgtgattaagcctagttctgtatctcgtaattgtgaaaaaatttgaatcatttccctctagaccatttcatttcaaacttatactttgtttagtctagccagttgaccaaaaagaaagaaaataaaaggaaaaagatactcggccagatgtcgacg

SEQ ID NO: 23 - DNA, 1123 bp, Homo sapiens
  Incyte ID NO 7521726CB1
tatggcaggctgcgtcccactgctccagggcctggtcctggtcctcgccctccatcgtgtggagccctcagtatttctcccggcctccaaagcaaacgacgttctggtgaggtggaagcgtgcgggctcctatcttctggaagaactcttcgagggaaacttggaaaaagaatgttatgaagaaacctgtgtctatgaagaagcaagagaagtgtttgaaaatgaagtagtcactgatgaattctggagacgatataagggcggctccccgtgcatctcccagccctgcctccacaacggctcttgccaggacagcatctggggctacacctgcacctgctcccccggctatgagggcagcaactgcgagctggctaaaaatgaatgtcacccagagcggactgatgggtgtcaacacttctgcctcccaggacaggaatcctacacgtgcagctgtgctcagggctacaggcttggtgaggaccacaaacagtgtgtgccccacgaccagtgtgcctgcggggtgctgacctctgagaagcgtgcaccggatctacaggacctcccgtggcagaacgagccaagacccgctgatgatcaagataacgcacgtccatgtgcacatgcggtatgacgcggacgcgggggagaatgacctgtcactgctggagctggagtggcccatccagtgcccaggtgcggggctccccgtgtgcacccctgagaaagacttcgctgagcacctcctcatcccacgcaccaggggcctcctcagcggctgggcacgcaatggcactgacctgggcaactcgctgaccacgcggcctgtcacacttgtggagggggaggagtgcgggcaggtcctgaatgtgactgtcaccaccaggacctactgtgagagaagcagcgtggcggccatgcactggatggatggaagtgtggtcaccagagaacacagaggctcctggtttctcacgggggtcctgggctcgcagccagtaggagggcaggctcacatggtccttgtcaccaaggtctccaggtactcactctggtttaaacagatcatgaactaactgaaactcagctagccagaatgaacaacacaaccggaagcgggata

SEQ ID NO: 24 - DNA, 780 bp, Homo sapiens
  Incyte ID NO 7523383CB1
tatcatggccaaggactttcaagatatccagcagctgagctcggaggaaaatgaccatcctttccatcaaggtgcacagctgcaagccgagctgcggagcctgaaggaagctttcagcaacttctcctcgagcaccctgacggaggtccaggcaatcagcacccacggaggcagcgtgggtgacaagatcacatccctaggagccaagctggagaaacagcagcaggacctgaaagcagatcacgatgccctgctcttccatctgaagcacttccccgtggacctgcgcttcgtggcctgccagatggagctcctccacagcaacggctcccaaaggacctgctgccccgtcaactgggtggagcaccaaggcagctgctactggttctctcactccgggaaggcctgggctgaggcggagaagtactgccagctggagaacgcacacctggtggtcatcaactcctgggaggagcagaaattcattgtacaacacacgaaccccttcaatacctggataggtctcacggacagtgatggctcttggaaatgggtggatggcacagactataggcacaactacaagaactgggctgtcactcagccagataattggcacgggcacgagctgggtggaagtgaagactgtgttgaagtccagccggatggccgctggaacgatgacttctgcctgcaggtgtaccgctgggtgtgtgggaaaaggcggaatgccaccggcgaggtggcctgaccccagcacacctctggctaacccataca

SEQ ID NO: 25 - DNA, 1418 bp, Homo sapiens
  Incyte ID NO 7522027CB1
tatggcaggctgcgtcccactgctccagggcctggtcctggtcctcgccctccatcgtgtggagccctcagtatttctcccggcctccaaagcaaacgacgttctggtgaggtggaagcgtgcgggctcctatcttctggaagaactcttcgagggaaacttggaaaaagaatgttatgaagaaatctgtgtctatgaagaagcaagagaagtgtttgaaaatgaagtagtcactgatgaattctggagacgatataagggtaagtggtttccttcgtctcctcagaagtattaattcctcgggatgaggtgcgtgggtgggcttaggacgcttcacgaccccagctcagcggatgccaagcctctggctccaggacccacggtgtctctccagggaagggcagctccgactctccccaaggaccaggcggctccccgtgcatctcccagccctgcctccacaacggctcttgccaggacagcatctggggctacacctgcacctgctcccccggctatgagggcagcaactgcgagctggctaaaaatgaatgtcacccagagcggactgatgggtgtcaacacttctgcctcccaggacaggaatcctacacgtgcagctgtgctcagggctacaggcttggtgaggaccacaaacagtgtgtgccccacgaccagtgtgcctgcggggtgctgacctctgagaagcgtgcaccggatctacaggacctcccgtggcaggtaaagttaacaaattccgaaggaaaagacttctgtggtggtgttataatacgggaaaattttgtactgacaacagcaaaatgttcactgttacacaggaatattactgtaaaaacatattttaacagaacgagccaagacccgctgatgatcaagataacgcacgtccatgtgcacatgcggtatgacgcggacgcgggggagaatgacctgtcactgctggagctggagtggcccatccagtgcccaggtgcggggctccccgtgtgcacccctgagaaagacttcgctgagcacctcctcatcccacgcaccaggggcctcctcagcggctgggcacgcaatggcactgacctgggcaactcgctgaccacgcggcctgtcacacttgtggagggggaggagtgcgggcaggtcctgaatgtgactgtcaccaccaggacctactgtgagagaagcagcgtggcggccatgcactggatggatggaagtgtggtcaccagagaacacagaggctcctggtttctcacgggggtcctgggctcgcagccagtaggagggcaggctcacatggtccttgtcaccaaggtctccaggtactcactctggtttaaacagatcatgaactaactgaaactcagctagccagaatgaacaacacaaccggaagcgggata

SEQ ID NO: 26 - DNA, 2076 bp, Homo sapiens
  Incyte ID NO 7524406CB1
tagagcattgccttgttgctgacctttcagtatggggaggattggcatctcctgtctttttcctgcttcttggcattttagcatatctccagtagggtgtcctcgaattctgaataccaatttacgccaaattatggtcattagtgtcctggctgctgctgtttcacttttatatttttctgttgtcataatccgaaataagtatgggcgactaaccagagacaagaaatttcaaaggtacctggcacgagttaccgacattgaagctacagacaccaataaccccaatgtgagctatgggatcgtggtggactgtggtagcagtgggtctcgagtatttgtttactgctggccaaggcataatggcaatccacatgatctgttggatatcaggcaaatgagggataaaaaccgaaagccagtggtcatgaagataaaaccgggcatttcagaatttgctacctctccagagaaagtcagtgattacatttctccacttttgaactttgctgcagagcatgtgccacgggcaaaacacaaagagacacctctctacattctctgcacggctggaatgagaatcctccccgaaagccagcagaaagctattctggaagaccttctgaccgatatccccgtgcactttgactttctgttttctgactctcatgcagaagtaatttctgggaaacaagaaggtgtgtatgcttggattggcattaattttgtccttggacgatttgagcatattgaagatgatgatgaggccgttgtggaagttaacattcctggaagtgaaagcagcgaagccattgtccgtaaaaggacagcgggcattctcgacatgggcggcgtgtcgactcagatagcgtacgaagtccccaaaactgaagaagtagctaaaaacttgttagctgaatttaacttgggatgtgatgttcaccaaactgagcatgtgtatcgagtctatgtggccacgtttcttgggtttggtggcaatgctgctcgacagagatacgaagacagaatatttgccaacaccattcaaaagaacaggctcctgggtaaacagactggtctgactcctgatatgccgtacttggacccctgcctacccctagacattaaagatgaaatccagcaaaatggacaaaccatatacctacgagggactggagactttgacctgtgtcgagagactatccagcctttcatgaataaaacaaacgagacccagacttccctcaatggggtctaccagcccccaattcacttccagaacagtgaattctatggcttctccgaattctactactgcaccgaggatgtgttacgaatggggggagactacaatgctgctaaatttactaaagctgcaaaggattactgtgcaacaaagtggtccattttgcgggaacgctttgaccgaggactgtacgcctctcatgctgacctccacaggcttaagtgaactgctccatgttgtgggaccagggatgtgaagcaagtacatcaaccttgaaacacgcgcagttggtttgggagagccggcctgaggaagcccctgccccaaggctgcccacagagggaaggattgtgtgtgtgtgtgtgtgtgtgccatcttgacagagtgagtcagtctgacttgcctttgtgcttgccgtttgtaggtatcagtgcttcaaatcggcctggatgtttgaggtgtttcataggggcttttcgtttcctgtcagctataaaagcttaaagactgccttgcaagtttacgacaaggaggttcagtggacccttggagccatcctctacaggacccgctttctaccattaagagacatccagcaggaggccttccgagccagtcacacccactggcggggcgtttcctttgtctacaaccactacctgttctctggctgcttcctggtggtgctgctggccatcctgctgtacctgctgcggctgcggcgcatccacaggcgcactccccggagcagctcggccgccgccctctggatggaggagggccttcccgcccagaatgccccagggaccttgtgatccagctcacagcta

SEQ ID NO: 27 - DNA, 783 bp, Homo sapiens
  Incyte ID NO 7524922CB1
tatgagtgactccaaggaaccaagggtgcagcagctgggcctcctggtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacaactccgtcaccgcctgccaggaagtgagggcccagctcgtcgtaatcaaaactgctgaggagcagcttccagcggtactggaacagtggagaacccaacaatagcgggaatgaagactgtgcggaatttagtggcagtggctggaacgacaatcgatgtgacgttgacaattactggatctgcaaaaagccagcaccctgcttcagagacgaataga

SEQ ID NO: 28 - DNA, 1115 bp, Homo sapiens
  Incyte ID NO 7524936CB1
tatgagtgactccaaggaaccaagggtgcagcagctgggcctcctggaagatccaacaaccagtggcatcagactttttccaagagactttcaattccagcagatacatggccacaagagctctacagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactggaacagtggagaacccaacaatagcgggaatgaagactgtgcggaatttagtggcagtggctggaacgacaatcgatgtgacgttgacaattactggatctgcaaaaagccagcacctcgcttcagagacgaataga

SEQ ID NO: 29 - DNA, 528 bp, Homo sapiens
  Incyte ID NO 7512039CB1
aaacactcctgaggagaaagaaagagagggagggagagaaaaagagagagagagaaacaaaaaaccaaagagagagaaaaaatgaattcatctaaatcatctgaaacacaatgcacagagagaggatgcttctcttcccaaatgttcttatggactgttgctgggatccccatcctatttctcagtgcctgtttcatcaccagatgtgttgtgacatttcgcatctttcaaacctgtgatgagaaaaagtttcagctacctgagaatttcacagagctctcctgctacaattatggatcagcttctgggatgtaggggagcccaacaacatagctaccctggaggactgtgccaccatgagagactcttcaaacccaaggcaaaattggaatgatgtaacctgtttcctcaattattttcggatttgtgaaatggtaggaataaatcctttgaacaaaggaaaatctctttaagaacagaaggcacaactcaaatgtgtaaagaaggaagagcaagaacattggccac

SEQ ID NO: 30 - DNA, 1365 bp, Homo sapiens
  Incyte ID NO 7512576CB1
cgccttcacgatgccggcggtcagtggtccaggtcccttattctgccttctcctcctgctcctggacccccacagccctgagacggggtgtcctcctctacgcaggtttgagtacaagctcagcttcaaaggcccaaggctggcattgcctggggctggaatacccttctggagccatcatggagacgccatcctgggcctggaggaagtgcggctgacgccatccatgaggaaccggagtggcgccgtgtggagcagggcctctgtccccttctctgcctgggaagtagaggtgcagatgagggtgacgggactggggcgccggggagcccagggcatggccgtgtggtacacccggggcaggggccatgtaggctctgtccttggggggctggcttcgtgggacggcatcgggatcttctttgactctccggcagaggatactcaggacagtcctgccatccgtgtgctggccagcgacgggcacatcccctctgagcagcctggggatggagctagccaagggctgggctcctgtcattgggacttccggaaccggccacaccccttcagagcacggatcacctactgggggcagaggctgcgcatgtccttgaacagtggcctcactcccagtgatccagatgatcatgatgtcctgtccttcctgaccttcagcctgagtgagcccagcccagaggttccccctcagcccttcctggagatgcagcagctccgcctggcgaggcagctggaagggctgtgggcaaggctgggcttgggcaccagggaggatgtaactccaaaatcagactctgaagctcaaggagaaggggaaaggctctttgacctggaggagacgctgggcagacaccgccggatcctgcaggctctgcggggtctctccaagcagctggcccaggctgagagacaatggaagaagcagctggggcccccaggccaagccaggcctgacggaggctgggccctggatgcttcctgccagattccatccaccccagggaggggtggccacctctccatgtcactcaataaggactctgccaaggtcggtgccctgctccatggacagtggactctgctccaggccctgcaagagatgagcaggcaggagctgaacaagagccttcaggagtgtctgtccacaggcagccttcctctgggtcctgcaccacacacccccagggccctggggattctgatgaggcagcctctccctgccagcatgcctgcctgacccacctcagagcctgctttgcatcactgggaagcaggcagtgtcttgggtgggggcttggtcagtatcctctccgtctgtgtacccagctgccacgaactgagctt

SEQ ID NO: 31 - DNA, 1343 bp, Homo sapiens
  Incyte ID NO 7514864CB1
taagtagccgcaggcatggcggcggctatgccgcttgctctgctcgtcctgttgctcctggggcccggcggctggtgccttgcagaacccccacgcgacagcctgcgggaggaacttgtcatcaccccgctgccttccggggacgtagccgccacattccagttccgcacgcgctgggattcggagcttcagcgggaaggaggcctctctgtgctgctgaaggcagatcgcttgttccacaccagctaccactcccaggcagtgcatatccgccctgtttgcagaaatgcacgctgtactagcatctcctgggagctgaggcagaccctgtcagttgtatttgatgccttcatcgcggggcagggaaagaaagactggtccctcttccggatgttctcccgaaccctcacggagccctgccccctggcttcagagagccgagtctatgtggacatcaccacctacaaccaggacaacgagacattagaggtgcacccacccccgaccactacatatcaggacgtcatcctaggcactcggaagacctatgccatctatgacttgcttgacaccgccatgatcaacaactctcgaaacctcaacatccagctcaagtggaagagacccccagagaatgaggcccccccagtgcccttcctgcgtgcccagcggtacgtgagtggctatgggctgcagaagggggagctgagcacactgctgtacaacacccacccataccgggccttcccggtgctgctgctggacaccgtaccctggtatctgcggctgtatgtgcacaccctcaccatcacctccaagggcaaggagaacaaaccaagttacatccactaccagcctgcccaggaccggctgcaaccccacctcctggagatgctgattcagctgccggccaactcagtcaccaaggtttccatccagtttgagcgggcgctgctgaagtggaccgagtacacgccagatcctaaccatggcttctatgtcagcccatctgtcctcagcgcccttgtgcccagcatggtagcagccaagccagtggactgggaagagagtcccctcttcaacagcctgttcccagtctctgatggctctaactactttgtgcggctctacacggagccgctgctggtgaacctgccgacaccggacttcagcatgccctacaacgtgatctgcctcacgtgcactgtggtggccgtgtgctacggctccttctacaatctcctcacccgaaccttccacatcgaggagccccgcacaggtggcctggccaagcggctggccaaccttatccggcgcgcccgaggtgtccccccactctgata

SEQ ID NO: 32 - DNA, 1840 bp, Homo sapiens
  Incyte ID NO 8266965CB1
agcttctacccggagctggggccctacagctcccgggaccccgaagtgctgcgggagcatatgacccagctcaaggaagccgccatcggcgtcctggtcctgtcctggtacccacctggcatggctgatgataacggggagccctcagatgacctggtgcccgccattctggacaccgcccatcagtacagcatccaggtggccttccacatccaaccctacaagggccgggatgacatcactgtacatgacaacatcaagtacatcattgacacgtatggctcccatggtgcattttaccgctataagaacagcatgggcaagagcctcccactcttttatatctacgactcatacctgacgtcccctgaggcctgggcccacctcctgacaccaaacgggccccattcgatccgcaacacgccctacgatggggtcttcatagcgctgctggtggaggagggccacacccacgatatcctggccgccggatttgacggcatgtacacctactttgcctccaatggtttctcctttggttcttcccatcagaactggaaagctgtgaagaacttttgtgatgccaacaacctcatgttcatccccagtgtggggcctggctacatagacaccagcattcggccctggaacaaccacaatacgcgcaacagggtcaatggcaagtactatgagacggccctgcaggcggccctgacagtgaggcccgagatcgtttccattacctccttcaatgagtggcacgagggcacccagattgagaaggccattcccaagaagacacccacccgcctgtatttggactacctgcctcaccagcccagcctgtacctggagctgacacgccgctgggcggagcacttcatcaaagagaaggagcagtggctcatgtgaggggcctgtaaatgggcgtgaggtgctgatgtccttgccttgctggaagatgtcaccatgtggggttcagctgaggttgtagccactcactcgttcccaggtcagaggtcagcagatgggtgtttctgggtgggccgtcaggcatgggcctgtgcaacacagagcccgttcctcaggcgagtggtgctgaggtgctctgtggtgatgggaacggcagaggctggcaggtgactgaacttagcccagggcagctccacatcctgggaacactttcatgtaaccccttctagttttgaactctgcagcaggctggtgctgtgtagtggccacccagtcaccacccttggaagggtgtcagggtctgggctcgcatgcaccctgctcccttctgccagcctgcatcctctcctcaggcctccttcccacatcatctgtcttctctaagttagggaaccatatttgagactttcaaaaagggaacttctaggtttaagctcctcccagtagattcctgaacccaattatcaggaaatcatcctgagcactcacaggttcatttaacactcactcatcaagcaccttcctatgtgctaggtgctggggaaaacttgaccaaggaagagttcctgtcctgaagcttccaggacccagctttccttttctggtgtggccttgtagctagtgcctgggcacaggtgtttttctttttgcagttttacctagtgctgggagttcagttctttttcctctagaaaaatacctctgtgctccagagcctaatttttcccagatgcatatttagctctagggagaggactaggaggaaatccccctccctttagctgcctgaactgactgaggcccactcactagagccatgttcagtgctactgtgattagtagtaattaaatatgaactggtattctc

SEQ ID NO: 33 - DNA, 523 bp, Homo sapiens
  Incyte ID NO 7515124CB1
agagccagatcgtcatcatgtctgcattgtggctgctgctgggcctccttgccctgatgggagttcgtgcttctgaaaggctggctgaaatagacatgccatacctcctgaaatatcaacccatgatgcaaaccattggccaaaagtactgcatggatcctgccgtgatcgctggtgtcttgtccaggaagtctcccggtgacaaaattctggtcaacatgggcgataggactagcatggtgcaggaccctggctctcaagctcccacatcctggattagtgagtctcaggtttcccagacaactgaagttctgactactagaatcaaagaaatccagaggaggtttccaacctggacccctgaccagtacctgagaggtggactctgtgcctacagtgggggtgctggctatgtccgaagcagccaggacctgagctgtgacttctgcaatgatgtccttgcacgagccaagtacctcaagagacatggcttctaacatctcagatgaaacccaagaccata

SEQ ID NO: 34 - DNA, 924 bp, Homo sapiens
  Incyte ID NO 7514570CB1
tatgcatgacagtaacaatgtggagaaagacattacaccatctgaattgcctgcaaacccagcaataagagctaactgccatcaagagccatcagtatgtcttcaagctgcatgcccagaaagctggattggttttcaaagaaagtgtttctatttttctgatgacaccaagaactggacatcaagtcagaggttttgtgactcacaagatgctgatcttgctcaggttgaaagcttccaggaactgaatttcctgttgagatataaaggcccatctgatcactggattgggctgagcagagaacaaggccaaccatggaaatggataaatggtactgaatggacaagacagtttcctatcctgggagcaggagagtgtgcctatttgaatgacaaaggtgccagtagtgccaggcactacacagagaggaagtggatttgttccaaatcagatatacatgtctagatgttacagcaaagccccaactaatctttagaagcatattggaactgataactccattttaaaatgagcaaagaatttatttcttataccaacaggtatatgaaaatatgctcaatatcactaataactgggaaaatacaaatcaaaatcatagtaaaatattacctgttttcatggtgctaatattacctgttctcccactgctaatgacatacccgagactgagtaatttataaataaaagagatttaattgactcatagttccacatggctggggaggtcttgcaatcatgacagaaggcaaatgggaagcaaagtcatgtcttatgtggtggcaggcagggggacttgtgcacaggaactcctatttatacaaccatcagatatcttgagacaagaacagtatggggctccctggtgtgattccgtcctgcgcggctgttctctggagcagcat

SEQ ID NO: 35 - DNA, 1346 bp, Homo sapiens
  Incyte ID NO 7515114CB1
tagacttcaggcgccttcacgatgccggcggtcagtggtccaggtcccttattctgccttctcctcctgctcctggacccccacagccctgagacggggtgtcctcctctacgcaggtttgagtacaagctcagcttcaaaggcccaaggctggcattgcctggggctggaatacccttctggagccatcatggagacgccatcctgggcctggaggaagtgcggctgacgccatccatgaggaaccggagtggcgccgtgtggagcagggcctctgtccccttctctgcctgggaagtagaggtgcagatgagggtgacgggactggggcgccggggagcccagggcatggccgtgtggtacacccggggcaggggccatgtaggctctgtccttggggggctggcttcgtgggacggcatcgggatcttctttgactctccggcagaggatactcaggacagtcctgccatccgtgtgctggccagcgacgggcacatcccctctgagcagcctggggatggagctagccaagggctgggctcctgtcattgggacttccggaaccggccacaccccttcagagcacggatcacctactgggggcagaggctgcgcatgtccttgaacagtggcctcactcccagtgatccaggtgagttctgtgtggatgtggggcccctgcttttggtccctggaggtttctttggggtctcagcagccaccggcaccctggcaggtgaggatcccactggacaggttccccctcagcccttcctggagatgcagcagctccgcctggcgaggcagctggaagggctgtgggcaaggctgggcttgggcaccagggaggatgtaactccaaaatcagactctgaagctcaaggagaaggggaaaggctctttgacctggaggagacgctgggcagacaccgccggatcctgcaggctctgcggggtctctccaagcagctggcccaggctgagagacaatggaagaagcagctggggcccccaggccaaaccaggcctgacggaggctgggccctggatgcttcctgccagattccatccaccccagggaggggtggccacctctccatgtcactcaataaggactctgccaaggtcggtgccctgctccatggacagtggactctgctccaggccctgcaagagatgagcaggcaggagctgaacaagagccttcaggagtgtctgtccacaggcagccttcctctgggtcctgcaccacacacccccagggccctggggattctgaggaggcagcctctccctgccagcatgcctgcctgacccacctcagagcctgctttgcatcacta

SEQ ID NO: 36 - DNA, 1379 bp, Homo sapiens
  Incyte ID NO 7515136CB1
tagacttcaggcgccttcacgatgccggcggtcagtggtccaggtcccttattctgccttctcctcctgctcctggacccccacagccctgagacggggtgtcctcctctacgcaggtttgagtacaagctcagcttcaaaggcccaaggctggcattgcctggggctggaatacccttctggagccatcatggagacgccatcctgggcctggaggaagtgcggctgacgccatccatgaggaaccggagtggcgccgtgtggagcagggcctctgtccccttctctgcctgggaagtagaggtgcagatgagggtgacgggactggggcgccggggagcccagggcatggccgtgtggtacacccggggcaggggccatgtaggctctgtccttggggggctggcttcgtgggacggcatcgggatcttctttgactctccggcagaggatactcaggacagtcctgccatccgtgtgctggccagcgacgggcacatcccctctgagcagcctggggatggagctagccaagggctgggctcctgtcattgggacttccggaaccggccacaccccttcagagcacggatcacctactgggggcagaggctgcgcatgtccttgaacagtggcctcactcccagtgatccaggtgagttctgtgtggatgtggggcccctgcttttggtccctggaggtttctttggggtctcagcagccaccggcaccctggcagatgatcatgatgtcctgtccttcctgaccttcagcctgagtgagcccagcccagaggttccccctcagcccttcctggagatgcagcagctccgcctggcgaggcagctggaagggctgtgggcaaggctgggcttgggcaccagggaggatgtaactccaaaatcagactctgaagctcaaggagaaggggaaaggctctttgacctggaggagacgctgggcagacaccgccggatcctgcaggctctgcggggtctctccaagcagctggcccaggctgagagacaatggaagaagcagctggggcccccaggccaagccaggcctgacggaggctgggccctggatgcttcctgccagattccatccaccccagggaggggtggccacctctccatgtcactcaataaggactctgccaaggtcggtgccctgctccatggacagtggactctgctccgggccctgcaagagatgaggcaggagctgaacaagagccttcaggagtgtctgtccacaggcagccttcctctgggtcctgcaccacacacccccagggccctggggattctgaggaggcagcctctccctgccagcatgcctgcctgacccacctcagagcctgctttgcatcacta

SEQ ID NO: 37 - DNA, 999 bp, Homo sapiens
  Incyte ID NO 7515308CB1
gaaagatttgaaacagattttctgaagaaagcagaagctctcttcccattatgacttcggaaatcacttatgctgaagtgaggttcaaaaatgaattcaagtcctcaggcatcaacacagcctcttctgcagttttctttcaaaaatattctcagcttcttgaaaaaaagactacaaaagagctggttcatacaacattggagtgtgtgaaaaaaaatatgcccgtggaagagacagcctggagctgttgcccaaagaattggaagtcatttagttccaactgctactttatttctactgaatcagcatcttggcaagacagtgagaaggactgtgctagaatggaggctcacctgctggtgataaacactcaagaagagcaggatttcatcttccagaatctgcaagaagaatctgcttattttgtggggctctcagatccagaaggtcagcgacattggcaatgggttgatcagacaccatacaatgaaagttccgcattctggcatccacgtgagcccagtgatcccaatgagcgctgcgttgtgctaaattttcgtaaatcacccaaaagatggggctggaatgatgttaattgtcttggtcctcaaaggtcagtttgtgagatgatgaagatccacttatgaactgaacattctccatgaacaggtggttggattggtatctgtcattgtagggatagataataagctcttcttattcatgtgtaagggaggtccatagaatttaggtggtctgtcaactattctacttatgagagaattggtctgtacattgactgattcactttttcataaagtgagcatttattgagcattttttcatgtgccagagcctgtactggaggcccccattgtgcacacatggagagaacatgagtctctcttaatttttatctggttgctaaagaattatttaccaataaaattatatgatgtggtgtctcctcctgtctggttttctcttcatccctactat

SEQ ID NO: 38 - DNA, 1072 bp, Homo sapiens
  Incyte ID NO 7516738CB1
tagtttgcttggagctcctggggcctaacaaaaagaaacctgccatgctgctcttcctcctctctgcactggtcctgctcacacagcccctgggctacctggaagcagaaatgaagacctactcccacagaacaatgcccagtgcttgcaccctggtcatgtgtagctcagtggagagtggcctgcctggtcgcgatggacgggatgggagagagggccctcggggcgagaagggggacccaggtttgccaggagctgcagggcaagcagggatgcctggacaagctggcccagttgggcccaaaggggacaatggctctgttggagaacctggaccaaagggagacactgggccaagtggagaagtaggtgccccaggcatgcagggctcggcaggggcaagaggcctcgcaggccctaagggagagcgaggtgtccctggtgagcgtggagtccctggaaacgcaggggcagcagggtctgctggagccatgggtccccagggaagtccaggtgccaggggacccccgggattgaagggggacaaaggcattcctggagacaaaggagcaaagggagaaagtgggcttccagatgttgcttctctgaggcagcaggttgaggccttacagggacaagtacagcacctccaggctgctttctctcagtataagaaagttgagctcttcccaaatggccaaagtgtcggggagaagattttcaagacagcaggctttgtaaaaccatttacggaggcacagctgctgtgcacacaggctggtggacagttggcctctccacgctctgccgctgagaatgccgccttgcaacagctggtcgtagctaagaacgaggctgctttcctgagcatgactgattccaagacagagggcaagttcacctaccccacaggagagtccctggtctattccaactgggccccaggggagcccaacgatgatggcgggtcagaggactgtgtggagatcttcaccaatggcaagtggaatgacagggcttgtggagaaaagcgtcttgtggtctgcgagttctgagccaactggggtgggta

SEQ ID NO: 39 - DNA, 872 bp, Homo sapiens
  Incyte ID NO 7518619CB1
tagactccaatcgacaagaagctggaaaagaatgatgttgtccttaaacaacctacagaatatcatctataacccggtaatcccgtatgttggcaccattcccgatcagctggatcctggaactttgattgtgatatgtgggcatgttcctagtgacgcagacagattccaggtggatctgcagaatggcagcagtgtgaaacctcgagccgatgtggcctttcatttcaatcctcgtttcaaaagggccggctgcattgtttgcaatactttgataaatgaaaaatggggacgggaagagatcacctatgacacgcctttcaaaagagaaaagtcttttgagatcgtgattatggtgctaaaggacaaattccaggttccaaagtctggcacgccccagcttagcctgccattcgctgcaaggttgaacacccccatgggccctggacgaactgtcgtcgttaaaggagaagtgaatgcaaatgccaaaagctttaatgttgacctactagcaggaaaatcaaaggatattgctctacacttgaacccacgcctgaatattaaagcatttgtaagaaattcttttcttcaggagtcctggggagaagaagagagaaatattacctctttcccatttagtcctgggatgtactttgagatgataatttactgtgatgttagagaattcaaggttgcagtaaatggcgtacacagcctggagtacaaacacagatttaaagagctcagcagtattgacacgctggaaattaatggagacatccacttactggaagtaaggagctggtagcctacctacacagctgctacaaaaaccaaaatacagaatggcttctgtgatactggccttgcta

SEQ ID NO: 40 - DNA, 864 bp, Homo sapiens
  Incyte ID NO 7513061CB1
aaatgacttaaagccaccagctaattgggcttaatcatttggactcagttgactcttcccctacccctacccatgcctaaaccaaagaaaggatcatcccacatttacctagcacaaagaaatctactcttctgctctttctaggactgctaaaggccatgggaactggacacctggatgctgcagaggaagggcaaagctcaacatcaacttggacagtttgccaacctgtttgtgagattgctgatttgctccttaagcaagagattcactgccgctaagcatggctcagaccaactcgttcttcatgctgatctcctccctgatgttcctgtctctgagccaaggccaggagtcccagacagagctgcctaatccccgaatcagctgcccagaaggcaccaatgcctatcgctcctactgctactactttaatgaagaccctgagacctgggttgatgcagatctctattgccagaacatgaattcaggcaacctggtgtctgtgctcacccaggcggagggtgccttcgtggcctcactgattaaggagagtagcactgatgacagcaatgtctggattggcctccatgacccaaaaaaggattcaagaaatggaaggatgaatcttgtgagaagaagttctcctttgtttgcaagttcaaaaactagaggaagctgaaaaatggatgtctagaactggtcctgcaattactatgaagtcaaaaattaaactagactatgtctccaactcagttcagaccatctcctccctaatgagtttgcatcgctgatcttcagtaccttcacctgtctcagtctctagagccctgaaaaataaaaacaaacttatttttaaaaaaa